(19) United States
(12) Patent Application Publication
(10) Pub. No.: US 2024/0400960 A1
(43) Pub. Date: Dec. 5, 2024

(54) ALGAL CO-CULTURE SYSTEM

(71) Applicants: The Trustees of Boston College, Chestnut Hill, MA (US); Worcester Polytechnic Institute, Worcester, MA (US)

(72) Inventors: Richard THYDEN, Sutton, MA (US); Glenn R. GAUDETTE, Holden, MA (US); Tanja DOMINKO, Jefferson, MA (US); Luke R. PERREAULT, Blackstone, MA (US)

(21) Appl. No.: 18/677,780

(22) Filed: May 29, 2024

Related U.S. Application Data
(60) Provisional application No. 63/505,024, filed on May 30, 2023.

(51) Int. Cl. C12M 1/00; C12M 1/12; C12M 1/34

(52) U.S. Cl. CPC C12M 23/58; C12M 21/02; C12M 25/02; C12M 41/12

(57) ABSTRACT

Mammalian cell production systems are disclosed herein wherein a living mammalian cell culture including a mammalian growth medium operative is co-cultured with a living algae cell culture including an algae growth medium operative to feed nutrients and carbon dioxide to a plurality of algae cells and to transport an algae waste from the plurality of algae cells. The living algae cell culture produces an algae waste with one or more nutrients and oxygen operative to feed the mammalian cell culture, while the living mammalian cell culture produces a mammalian waste with one or more nutrients and carbon dioxide operative to feed the algae cell culture. The systems are capable of a continuous operation at normal mammalian temperature and pH, thereby removing a need to cool down the algae growth medium and/or heat up the algae growth medium for a treatment of algae or for a replenishment of algae.

### CROSS-REFERENCE TO RELATED APPLICATIONS

This application claims the benefit of priority to United States (US) provisional application No. 63/505,024, filed May 30, 2023, the disclosure of which is incorporated by reference as if fully set forth herein in its entirety.

### FIELD OF THE INVENTION

Example embodiments of the present invention relate to methods to improve efficiencies of culturing adherent or nonadherent cells by using a co-culture of the adherent or non adherent cells with other cells such as algae cells. The various improvements in efficiencies can include recycling of cell culture media, green chemistry including far less environmental impact (e.g., climate change), and far less waste production compared to animal production of mammalian cells (e.g., waste from meat production), and sustainable methods that use less energy.

### BACKGROUND OF THE INVENTION

Global demand for meat has led to factory farming. In general, factory farming provides various systems for raising fast-growing animals in a concentrated area of confinement. Also known as concentrated animal feeding operations (CAFOs), factory farming has developed to meet the massive global demand for various animal meats, fish, eggs, and milk. CAFOs can be harmful to the environment. Wastewater treatment plants cannot handle the quantity of liquid waste from CAFOs, therefore much of it is released to the environment. The vast amounts of animal wastes that are produced by the many thousands of animals leads to high concentrations of ammonia in the air both in and around factory farms. The colorless ammonia gas has a strong smell and irritates the eyes and lungs of both people and animals.

CAFOs do not necessarily reduce the global use of farmland. Animal agriculture uses the majority of global farmland but, compared to plant production, produces far less percentage of edible calories and not even half of the world's protein. Meanwhile, vast areas of forests are cleared to grow corn and soy to feed factory-farmed animals. The process is known as de-forestation. Shifting human diets from meat and other animal products to plant-based diets has a great potential for reducing carbon footprints and mitigating climate change, as well as improving overall human health, according to a report from the Climate Change 2022: Mitigation of Climate Change, which is a report from the United Nations' Intergovernmental Panel on Climate Change¹.

The authors of Climate Change 20221 state that studies have demonstrated that a shift to plant-based diets rich in grains, legumes (i.e., pulses), nuts, fruits, and vegetables could lead to a substantial reduction of greenhouse gas emissions when compared to current dietary practices and patterns, such as meat consumption, in most industrialized countries. The benefits vastly extend beyond environmental benefits. The 2022 report states that other co-benefits include lowering the risk of cardiovascular disease, type 2 diabetes, and reducing mortality from diet-related noncommunicable diseases.

A report published in The Lancet in 2019² concluded that a dietary shift toward plant foods and away from animal products is vital for promoting the environmental health of planet Earth. The Lancet report asserts that projections for the future show that vegan and vegetarian diets were associated with the greatest reductions in greenhouse-gas emissions.

A global shift to a plant-based diet could reduce mortality and greenhouse gases caused by food production by 10% and 70%, respectively, by 2050. For example, a report from the United Nations Environment Programme³ states that animal products, both meat and dairy, in general require more resources and cause higher emissions than plant-based alternatives. The World Health Organization⁴ has stated that reducing livestock herds would also reduce emissions of methane, which is the second largest contributor to global warming after carbon dioxide. Currently, not everybody can change to a vegetarian diet or to a vegan diet. Thus, what is urgently needed are environmentally sustainable methods for mammalian cell culture including meat production that can provide healthier alternatives to the current global trends.

### BRIEF SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In the field of biotechnology, specifically in cell culture systems, there is a constant need for efficient and sustainable methods of cell growth and maintenance. Traditional cell culture systems often require separate environments for different types of cells, such as mammalian cells and algae cells. These environments need to be carefully controlled in terms of temperature, pH, and nutrient supply. Moreover, waste management is a significant concern in these systems as the waste produced by the cells can be harmful to the cells themselves if not properly managed. Additionally, the process of transferring growth mediums between different cell cultures can be complex and risk cross-contamination. Furthermore, the need for continuous operation of these systems without the need for frequent adjustments of the growth medium temperature is a challenge in the field.

In accordance with some embodiments, a biological cell production system is provided. The system includes a living cell culture with a growth medium that feeds nutrients and gases to cells and transports waste from the cells. A mass exchange system allows the growth medium to enter and/or exit the cell culture and prevents cross transfer of cells. The system can operate continuously at a certain temperature and pH range, eliminating the need to adjust the temperature of the growth medium for cell treatment or replenishment.

In some embodiments, a method of operating a biological cell production system is provided. The method involves providing a living cell culture and a mass exchange system, similar to the system described above. The system is operated continuously at a certain temperature and pH range, removing the need to adjust the temperature of the growth medium for cell treatment or replenishment.

In accordance with some embodiments, a mammalian cell production system is provided. The system includes a living mammalian cell culture and a living algae cell culture, both of which are operative to feed nutrients and oxygen to their respective cells and transport waste from them. The waste produced by the algae cell culture feeds the mammalian cell culture and vice versa. The system also includes a mass exchange system that allows the mammalian and algae growth mediums to enter and exit their respective cultures without cross transfer of cells. The system operates continuously at a temperature range of about 30° C. to 40° C. and a pH range of about 7 to 8, eliminating the need to cool down or heat up the algae growth medium for algae treatment or replenishment.

In another embodiment, a method for producing a tissue product is provided. The method involves obtaining the aforementioned mammalian cell production system, circulating the mammalian and algae growth mediums, and producing the tissue product by the growth of the mammalian cell culture, which is at least partially sustained by the algae cell culture.

In yet another embodiment, a method for recycling a mammalian cell culture medium is provided. The method involves obtaining the mammalian cell production system, extracting the algae waste from the algae cells, and obtaining a recycled mammalian cell culture medium from the algae waste.

Further as a brief introduction, some features of the technology disclosed herein can be summarized by the following list of features:

Feature 1: A mammalian cell production system, comprising: A) a living mammalian cell culture including a mammalian growth medium operative to feed nutrients and oxygen to a plurality of mammalian cells within the mammalian cell culture and to transport a mammalian waste from the plurality of mammalian cells; B) a living algae cell culture including an algae growth medium operative to feed nutrients and carbon dioxide to a plurality of algae cells within the algae cell culture and to transport an algae waste from the plurality of algae cells; wherein the living algae cell culture produces the algae waste with one or more nutrients and oxygen operative to feed the mammalian cell culture and wherein the living mammalian cell culture produces the mammalian waste with one or more nutrients and carbon dioxide operative to feed the algae cell culture; and C) a mass exchange system operative to allow the mammalian growth medium to enter and/or to exit the living algae cell culture and to allow the algae growth medium to enter and/or to exit the living mammalian cell culture without a cross transfer of mammalian cells and algae cells; wherein the system is capable of a continuous operation at a temperature in a range from about 30° C. to about 40° C. and a pH in a range from about 7 to about 8 thereby removing a need to cool down the algae growth medium and/or heat up the algae growth medium for a treatment of algae or for a replenishment of algae.

Feature 2: The mammalian cell production system of feature 1, wherein the system is capable of a continuous operation at a temperature at about 37° C.

Feature 3: The mammalian cell production system of feature 1, wherein the system is capable of a continuous operation at a pH at about 7.4.

Feature 4: The mammalian cell production system of feature 1, wherein the living algae cell culture is capable of receiving a light energy that is directed upon the plurality of algae cells.

Feature 4-5: The algal system or the system of feature 1 is capable of growth and waste removal in the absence of light, or in a combination of light and dark.

Feature 5: The mammalian cell production system of feature 1, wherein the mammalian waste comprises urea, carbon dioxide, lactic acid, ammonia, or a combination thereof.

Feature 6: The mammalian cell production system of feature 1, wherein the algae waste comprises glucose, oxygen, or a combination thereof.

Feature 7: The mammalian cell production system of feature 1, further comprising a mammalian cell substrate or a mammalian cell carrier operative to provide an adherent growth substrate for a plurality of mammalian cells.

Feature 8: The mammalian cell production system of feature 7, further comprising the plurality of mammalian cells include two or more mammalian cell types, and wherein the living mammalian cell culture is within a proliferation reactor that is in a fluid communication with the mass exchange system and with a conduit leading into a filter and a differentiation reactor; wherein the filter provides a passage of a differentiated growth seed comprising an adherent growth of the two or more cell types from the proliferation reactor and into the differentiation reactor.

Feature 9: The mammalian cell production system of feature 8, further comprising the differentiation reactor is in fluid communication with the mass exchange system; and the differentiation reactor provides a chamber for the differentiated growth see to grow into a differentiated cell populated scaffold; and the differentiation reactor includes an outlet operative to provide the differentiated cell populated scaffold.

Feature 10: The mammalian cell production system of feature 1, further comprising the mammalian cells include and/or are replaced by animal cells and/or vertebrate cells.

Feature 11: The mammalian cell production system of feature 1, further comprising the algae cells include and/or are replaced by one or more cells from a microorganism.

Feature 12: The mammalian cell production system of feature 1, further comprising one or more pumps, each of the one or more pumps capable of moving the mammalian growth medium and/or the algae growth medium.

Feature 13: The mammalian cell production system of feature 1, wherein one or more algae cells comprise Chlorella sorokiniana, Chlorococcum littorale, Chlorella vulgaris, Chlamydomonas reinhardtii, Parachlorella, Desmodesmus armatus, or a combination thereof.

Feature 14: The mammalian cell production system of feature 1, wherein one or more mammalian cells comprise stem cells, human cells, ruminants' cells, murine cells, porcine cells, bird cells, or a combination thereof.

Feature 15: The mammalian cell production system of feature 1, wherein the system is capable of a production of a mammal tissue suitable for a human consumption under one or more standards of human consumption.

Feature 15-5: The mammalian cell production system of feature 1, wherein the system is capable of a production of byproducts of cellular processes for pharmaceutical applications, a manufacturing of monoclonal antibodies, a making of enzymes, a production of genes for gene therapy, and/or for other biological production processes.

Feature 16: The mammalian cell production system of feature 15, wherein the production of a mammal tissue is a meat production; wherein the meat is suitable for human consumption; and wherein the meat production is provided with a lower waste emission into an environment when compared to a production of the same meat utilizing one or more living animals.

Feature 17: The mammalian cell production system of feature 1, wherein the mass exchange system includes a semi-permeable membrane, a filter, a zeolite, a hollow fiber, a filter, or a combination thereof.

Feature 18: A method for producing a tissue product, the method comprising the steps of: (1) obtaining a mammalian cell production system, comprising: A) a living mammalian cell culture including a mammalian growth medium operative to feed nutrients and oxygen to a plurality of mammalian cells within the mammalian cell culture and to transport a mammalian waste from the plurality of mammalian cells; B) a living algae cell culture including an algae growth medium operative to feed nutrients and carbon dioxide to a plurality of algae cells within the algae cell culture and to transport an algae waste from the plurality of algae cells; wherein the living algae cell culture produces the algae waste with one or more nutrients and oxygen operative to feed the mammalian cell culture and wherein the living mammalian cell culture produces the mammalian waste with one or more nutrients and carbon dioxide operative to feed the algae cell culture; C) a mass exchange system operative to allow the mammalian growth medium to enter and/or to exit the living algae cell culture and to allow the algae growth medium to enter and/or to exit the living mammalian cell culture without a cross transfer of mammalian cells and algae cells; (2) circulating the mammalian growth medium and the algae growth medium, whereby the mammalian growth medium and the algae growth medium enter and exit the mass exchange system; and whereby the tissue product is produced by a growth of the living mammalian cell culture that is at least partially sustained by the algae cell culture.

Feature 19: The method of feature 18, wherein the system is capable of a continuous operation at a temperature in a range from about 30° C. to about 40° C. and a pH in a range from about 7 to about 8 thereby removing a need to cool down the algae growth medium and/or heat up the algae growth medium for a treatment of algae or for a replenishment of algae.

Feature 20: The method of feature 18, wherein the system is capable of a continuous operation at a temperature at about 37° C.

Feature 21: The method of feature 18, wherein the system is capable of a continuous operation at a pH at about 7.4.

Feature 22: The method of feature 18, further comprising a mammalian cell substrate or a mammalian cell carrier operative to provide an adherent growth substrate for a plurality of mammalian cells.

Feature 23: The method of feature 22, further comprising the plurality of mammalian cells include two or more mammalian cell types, and wherein the living mammalian cell culture is within a proliferation reactor that is in a fluid communication with the mass exchange system and with a conduit leading into a filter and a differentiation reactor; wherein the filter provides a passage of a differentiated growth seed comprising an adherent growth of the two or more cell types from the proliferation reactor and into the differentiation reactor.

Feature 24: The method of feature 23, further comprising the differentiation reactor is in fluid communication with the mass exchange system; and the differentiation reactor provides a chamber for the differentiated growth see to grow into a differentiated cell populated scaffold; and the differentiation reactor includes an outlet operative to provide the differentiated cell populated scaffold.

Feature 25: The method of feature 18, wherein the living algae cell culture is capable of receiving a light energy that is directed upon the plurality of algae cells.

Feature 26: The method of feature 18, wherein the mammalian waste comprises urea, carbon dioxide, lactic acid, ammonia, or a combination thereof.

Feature 27: The method of feature 18, wherein the algae waste comprises glucose, oxygen, or a combination thereof.

Feature 28: The method of feature 18, further comprising the mammalian cells include and/or are replaced by animal cells and/or vertebrate cells.

Feature 29: The method of feature 18, further comprising the algae cells include and/or are replaced by one or more cells from a microorganism.

Feature 30: The method of feature 18, further comprising one or more pumps, each of the one or more pumps capable of moving the mammalian growth medium and/or the algae growth medium.

Feature 31: The method of feature 18, wherein one or more algae cells comprise Chlorella sorokiniana, Chlorococcum littorale, Chlorella vulgaris, Chlamydomonas reinhardtii, Parachlorella, Desmodesmus armatus, or a combination thereof.

Feature 32: The method of feature 18, wherein one or more mammalian cells comprise stem cells, human cells, ruminants' cells, murine cells, porcine cells, bird cells, or a combination thereof.

Feature 33: The method of feature 18, wherein the system is capable of a production of a mammal tissue suitable for a human consumption under one or more standards of human consumption.

Feature 34: The method of feature 33, wherein the production of a mammal tissue is a meat production; wherein the meat is suitable for human consumption; and wherein the meat production is provided with a lower waste emission into an environment when compared to a production of the same meat utilizing one or more living animals.

Feature 35: The method of feature 18, wherein the mass exchange system includes a semi-permeable membrane, a filter, a zeolite, a hollow fiber, a filter, or a combination thereof.

Feature 36: A method for recycling a mammalian cell culture medium, the method comprising the steps of: (1) obtaining a mammalian cell production system, comprising: A) a living mammalian cell culture including a mammalian growth medium operative to feed nutrients and oxygen to a plurality of mammalian cells within the mammalian cell culture and to transport a mammalian waste from the plurality of mammalian cells; B) a living algae cell culture operative to utilize the mammalian growth medium to feed nutrients and carbon dioxide to a plurality of algae cells within the algae cell culture and to transport an algae waste from the plurality of algae cells; wherein the living algae cell culture produces the algae waste with one or more nutrients and oxygen operative to feed the mammalian cell culture and wherein the living mammalian cell culture produces the mammalian waste with one or more nutrients and carbon dioxide operative to feed the algae cell culture; wherein the system is capable of a continuous operation at a temperature in a range from about 30° C. to about 40° C. and a pH in a range from about 7 to about 8 thereby removing a need to cool down the algae growth medium and/or heat up the algae growth medium for a treatment of algae or for a replenishment of algae; (2) extracting the algae waste from the plurality of algae cells; and whereby a recycled mammalian cell culture medium is obtained from the algae waste.

Feature 37: The method of feature 36, further comprising the step of: (1A) obtaining a mass exchange system operative to allow the mammalian growth medium to enter and/or to exit the living algae cell culture and to allow an algae growth medium to enter and/or to exit the living mammalian cell culture without a cross transfer of mammalian cells and algae cells.

Feature 38: The method of feature 36, wherein the system is capable of a continuous operation at a temperature at about 37° C.

Feature 39: The method of feature 36, wherein the system is capable of a continuous operation at a pH at about 7.4.

Feature 40: The method of feature 36, wherein the living algae cell culture is capable of receiving a light energy that is directed upon the plurality of algae cells.

Feature 41: The method of feature 36, wherein the mammalian waste comprises urea, carbon dioxide, lactic acid, ammonia, or a combination thereof.

Feature 42: The method of feature 36, wherein the algae waste comprises glucose, oxygen, or a combination thereof.

Feature 43: The method of feature 36, further comprising a mammalian cell substrate or a mammalian cell carrier operative to provide an adherent growth substrate for a plurality of mammalian cells.

Feature 44: The method of feature 43, further comprising the plurality of mammalian cells include two or more mammalian cell types, and wherein the living mammalian cell culture is within a proliferation reactor that is in a fluid communication with the mass exchange system and with a conduit leading into a filter and a differentiation reactor; wherein the filter provides a passage of a differentiated growth seed comprising an adherent growth of the two or more cell types from the proliferation reactor and into the differentiation reactor.

Feature 45: The method of feature 44, further comprising the differentiation reactor is in fluid communication with the mass exchange system; and the differentiation reactor provides a chamber for the differentiated growth see to grow into a differentiated cell populated scaffold; and the differentiation reactor includes an outlet operative to provide the differentiated cell populated scaffold.

Feature 46: The method of feature 36, further comprising the mammalian cells include and/or are replaced by animal cells and/or vertebrate cells.

Feature 47: The method of feature 36, further comprising the algae cells include and/or are replaced by one or more cells from a microorganism.

Feature 48: The method of feature 36, further comprising one or more pumps, each of the one or more pumps capable of moving the mammalian growth medium and/or the algae growth medium.

Feature 49: The method of feature 36, wherein one or more algae cells comprise Chlorella sorokiniana, Chlorococcum littorale, Chlorella vulgaris, Chlamydomonas reinhardtii, Parachlorella, Desmodesmus armatus, or a combination thereof.

Feature 50: The method of feature 36, wherein one or more mammalian cells comprise stem cells, human cells, ruminants' cells, murine cells, porcine cells, bird cells, or a combination thereof.

Feature 51: The method of feature 36, wherein the system is capable of a production of a mammal tissue suitable for a human consumption under one or more standards of human consumption.

Feature 52: The method of feature 51, wherein the production of a mammal tissue is a meat production; wherein the meat is suitable for human consumption; and wherein the meat production is provided with a lower waste emission into an environment when compared to a production of the same meat utilizing one or more living animals.

Feature 53: The method of feature 36, wherein the mass exchange system includes a semi-permeable membrane, a filter, a zeolite, a hollow fiber, a filter, or a combination thereof.

Other implementations are also described and recited herein. These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

Solely for the purpose of illustration, certain embodiments of the present invention are explained using examples in the drawings described below. It should be understood, however, that the invention is not limited to the precise arrangements, dimensions, and configurations shown. In the drawings:

FIG. 1A shows a chart of greenhouse gas emissions (GHG) by economic sector-.⁵

FIG. 1B shows a chart of energy consumed by economic sector in the US.⁶

FIG. 1C shows a chart of use of freshwater in the US.

FIG. 1D shows a chart illustrating a comparison of the origin of diseases infectious to humans.

FIG. 1E shows a chart of potential reduction of environmental impact of meat production by cellular agriculture.

FIG. 1F shows a chart illustrating the current cost to produce one pound of each meat product.

FIG. 1G shows an overall equation of aerobic respiration.

FIG. 1H shows an overall equation of photosynthesis.

FIG. 1I shows an overall equation of lactic acid fermentation.

FIG. 2A shows a diagrammatic representation of a finalized simple pump design according to some embodiments.

FIG. 2B shows a diagrammatic representation of the hourglass design according to some aspects of the technology.

FIG. 2C shows a diagrammatic representation of the teabag design.

FIG. 3A shows a plot summarizing a comparison of alae in Tris-Acetate-Phosphate medium (TAP) vs. Dulbecco's Modified Eagle Medium (DMEM).

FIG. 3B shows a photo of the two treatments of TAP and DMEM.

FIG. 3C shows at left, (A), C. reinhardtii culture after 48 hours with constant stir bar agitation and at right, (B), C. reinhardtii culture after 48 hours without any stir bar agitation.

FIG. 3D shows a graph of cells counts for the algae agitation experiment (n=2).

FIG. 3E shows an image of the algae with a darker brown color, which is indicative of heat stress or a lack of light.

FIG. 3F shows representative images (A-C) from the Transwell Hoechst stain (Left to right: no algae A, Algae 100:1 B, Algae 200:1 C).

FIG. 3G shows the estimated number of primary bovine satellite cells (PBSCs) in a Transwell after three days of co-culture at varying ratios.

FIG. 3H illustrates the estimated number of PBSCs per well in the three different ratios.

FIG. 3I shows co-culture with PBSCs and algae cells at (A) Day 1, (C) Day 2, (E) Day 3, and monoculture at (B) Day 1, (D) Day 2, (F) Day 3.

FIG. 3J shows Live/Dead stain of monoculture (A, B) and co-culture (C, D) at 8×.

FIG. 3K shows a comparison of the % dissolved oxygen in the monoculture and co-culture system over a 10-day period.

FIG. 3L shows a comparison of the pH in the monoculture and co-culture system over a 10-day period.

FIG. 4A shows a diagrammatic representation of a cross-section of the algae cells 135 flowing through a hollow fiber model.

FIG. 4B shows a diagrammatic representation of a process flow diagram of an overall hollow fiber reactor model.

FIG. 4C shows a model of oxygen diffusion in the lumen.

FIG. 4D shows pictures of Hoechst staining of the Transwell.

FIG. 4E shows the monoculture and co-culture dissolved oxygen (DO) vs. time plot.

FIG. 4F shows the monoculture and co-culture pH versus time.

FIG. 4G shows the mathematical formulas used to calculate the peclet number of the fiber.

FIG. 4H shows the mathematical equation used to calculate the shear rate.

FIG. 5A shows a diagrammatic representation of a simplified process for recycling spent animal cell culture media.

FIG. 5B shows a diagrammatic representation of a theoretical process for supporting suspension bioreactors with algal bioreactors including mammalian cell carrier 175 and mass exchange system 170.

FIG. 5C shows equation 1, which was utilized to calculate growth rates.

FIG. 6A shows a plot of growth of C. sorokiniana.

FIG. 6B shows a plot of C. sorokiniana light intensity and growth by day.

FIG. 6C shows a plot of C. sorokiniana light growth versus dark growth.

FIG. 6D shows a plot of C. sorokiniana light growth versus dark growth by day.

FIG. 6E shows a photo of flask cultures of C. sorokiniana.

FIG. 7A shows a plot of glucose consumption.

FIG. 7B shows a plot of ammonia generation.

FIG. 8A shows a plot of C. sorokiniana media type and growth.

FIG. 8B shows a graph of C. sorokiniana media type and growth by day.

FIG. 8C shows a microscopic brightfield image of C. sorokiniana.

FIG. 8D shows an autofluorescence of algal chlorophyll after 7 days growth in GM 649 nm/667 nm excitation/emission.

FIG. 9A shows an algae growth curve.

FIG. 9B shows an ammonia consumption curve.

FIG. 9C shows a glucose consumption curve.

FIG. 10A shows a photo of a C. sorokiniana filtration.

FIG. 10B shows QM7 cells stained using live (green cytoplasm or lighter cytoplasm)/dead (red nuclei) stain.

FIG. 10C shows a graph of cell viability, determined via colocalization of dead signal with nuclear stain, compared between QM7 cells cultured in GM and 8D-SGM after four days.

FIG. 10D shows an MTT Analysis of QM7 cells that were exposed to GM, 8D-SGM, and Alg-Treated-8D-SGM.

FIG. 10E shows Table 6.

FIG. 11 shows a diagrammatic representation of a proposed bioprocess.

FIG. 12A shows Chlorella vulgaris (BFS20), Parachlorella (PK25), and Desmodesmus armatus (DA25-HFL) in cultures.

FIG. 12B shows absorbance units (AU) at 750 nm.

FIG. 12C shows a plot of chlorophyl fluorescence using 440 nm and 680 nm.

FIG. 12D shows a plot of absorbance (750 nm) versus time (hours).

FIG. 12E shows a plot of algae count with absorbance at 670 nm on the X-axis.

FIG. 12F shows a plot of absorbance at 670 nm versus hours with different media.

FIG. 12G shows a plot of absorbance at 670 nm versus hours and concentration is at the right of the plot with different media.

FIG. 12H shows an image of filtering algae treated DMEM.

FIG. 12I shows fluorescent images of algae treated media (top), DMEM F12 and

10% FBS media (middle), and dead control (bottom).

FIG. 12J shows a plot illustrating culture media cytotoxicity.

FIG. 12K shows a plot of ammonia concentration with different growth media (GM) on the X-axis.

FIG. 13 shows a flow chart of an example method for a tissue (or meat) production.

It should be understood that while different numbers/numbering are/is sometimes used in some of the figures above to describe different embodiments and different aspects of the technology, any number from any figure can be inter-combined with a numbered aspect from any other figures. All trademarks, images, likenesses, words, and depictions in the drawings and the disclosure are plainly in fair use and are provided solely for the purposes of illustration of the invention in view of an urgent need to treat subjects as further discussed in detail below.

### DETAILED DESCRIPTION OF THE INVENTION

The subject innovation is now described in some instances, when necessary, with reference to the drawings. In the following description, for purposes of explanation, numerous specific details are set forth in order to provide a thorough understanding of the present invention. It may be evident, however, that the present invention may be practiced without these specific details. In other instances, well-known structures, methods, and devices are shown in block diagram form or with illustrations in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation. The term “including” can be interchanged with “comprising”.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention. The term “consisting essentially of” can also be exemplified by plain language provided in the claims.

As used herein, the term “reactor” can be used interchangeably with “culture system”.

The term “statistically significant” or “significantly” refers to statistical significance and generally means a two-standard deviation (2SD) or greater difference.

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, an agent or a therapeutic agent provided to a subject and suspected to be or involved in a treatment can be a small molecule less than 1000 MW or a large molecule not less than 1000 MW including biologics, oligonucleotides, peptides, oligosaccharides, and larger molecules. Any of the therapeutic agents disclosed herein can be used as or in combination with small molecules and/or large molecules as discussed herein.

Other terms are defined herein within the description of the various aspects of the invention.

### Algal Co-Culture System

The global demand for meat is expected to double in the next half-century, but current meat production practices pose significant hazards to the environment and human health. The emerging field of cellular agriculture has the potential to solve problems associated with traditional animal agriculture by culturing animal products in vitro. Cellular agriculture is potentially more environmentally sustainable, but there are hurdles to overcome in large-scale production. Cell culture media is the nutrient solution in which the cells are grown and is a limiting factor in the cost and environmental impact of large-scale cellular agriculture. There is a need to extend the lifetime of the media by removing metabolic waste products and replenishing the media with nutrients. This would reduce media-associated costs, water usage, and energy usage of the system. This initial project aimed to create a co-culture system of primary bovine satellite cells (PBSCs) and the microalga, Chlamydomonas reinhardtii, to extend the media lifetime and improve the sustainability of large-scale cellular agriculture. The success of this system was assessed by collecting data on dissolved oxygen concentrations, culture pH, and cell proliferation and viability. The data suggest that PBSCs can remain viable in co-culture with C. reinhardtii, and that the system increases dissolved oxygen and buffers the pH drop normally observed in animal cell culture. In a 200:1 ratio of C. reinhardtii to PBSCs grown in hypoxic conditions, the PBSCs were able to undergo one doubling in three days. Based on the pH data, the media lifetime was extended by 85%. This system should be further explored to optimize the media recycling potential of C. reinhardtii co-cultures.

Introduction, 1.0: When recognizing human contributions to climate change, animal agriculture cannot be ignored. Factory livestock contributes to 9% of carbon dioxide, 30% of methane, and 65% of nitrous oxide emissions⁷. This does not account for water, land, and energy use, or the safety and ethical concerns of factory farming. There is currently an effort to develop the field of cellular agriculture to improve global meat production practices. This field focuses on growing animal tissue in vitro by applying tissue engineering principles. One model predicts this approach could reduce greenhouse gas (GHG) emissions by 96%, land usage by 99%, water usage by 90%, and energy usage by 40% when compared to traditional beef production⁸. There is potential for this field to revolutionize our food production industry while improving our relationship with the environment. However, one of the most prohibitive factors in scale-up is cell culture media. The cost of media is high and volatile due to fluctuations in the price of fetal bovine serum (FBS), which makes consistent large-scale operations difficult⁹. While efforts are being made to move away from serum-based media with supplementation of growth factors and hormones, the quantities required for scale-up still warrant new cost reduction strategies.

Media needs to be replaced over time because animal cells take up the nutrients and produce toxic byproducts like lactic acid and ammonium¹⁰. In our environment, we consume nutrients and excrete waste, which other organisms use and eventually convert back into compounds we can use. The inventive team aimed to capture this symbiotic relationship in the lab to recycle the waste in spent media. The goal is that co-culturing complementary cell types in a micro-ecosystem will increase the lifetime of cell culture media. This would decrease the frequency of media replacement, thereby reducing media-associated costs. Co-cultures of microalgae and mammalian cells can reduce carbon dioxide and ammonium concentrations while increasing oxygen concentration¹¹. Algae culture can also increase the proliferation of mouse myoblasts in glucose and amino-acid deficient media¹². These studies show that co-culture micro-ecosystems can improve cell growth conditions. The inventive team decided to design a co-culture system for primary bovine satellite cells (PBSCs), which are used to make cultured beef.

After background research, and with relevant stakeholders in mind, the team determined objectives and constraints to guide the design process. To meet the societal need, the system must be scalable, allow proliferation and harvesting of PBSCs, and reduce the cost associated with cell culture media. For the cell culture system to work, the PBSCs must remain viable and retain their stem-phenotype, and the co-culture cell must remain viable and metabolically functional. The design was split into two aspects. The first is the co-culture system, which is largely defined by the specific cell species used to co-culture with PBSCs. After comparing the growth conditions and metabolic capabilities of multiple cell species, the microalga Chlamydomonas reinhardtii was chosen to be co-cultured with PBSCs. The second aspect of the design is scale-up, which is concerned with bioreactor designs that can incorporate the co-culture system for large-scale cultured meat production. The team determined design needs for the bioreactor and compared multiple conceptual designs to finally choose a hollow fiber bioreactor design that keeps the PBSC and C. reinhardtii cultures separate with a semi-permeable membrane.

The inventive team tested the prototype co-culture system to confirm that it fulfills the design objectives and constraints. The PBSCs and C. reinhardtii were first grown in monoculture to establish baseline growth conditions. Later experiments were focused on exploring the coculture's effect on PBSC proliferation and viability, the dissolved oxygen (DO) of the media, and the culture pH. The results indicated that co-culturing can promote PBSC proliferation and increase viability in hypoxic conditions. C. reinhardtii can remain viable in co-culture conditions, and it increases the DO while slowing the drop in pH. These data indicate that the coculture can increase media lifetime by 85%, thereby reducing the prohibitive media cost for scale-up. A theoretical model was created for a hollow-fiber culture design, although further work was necessary to prototype and validate the design. The team concluded from the results that C. reinhardtii co-cultures should continue to be explored as a path to reducing media costs for large-scale cellular agriculture.

### Literature Review

2.1 Current Animal Agriculture: Global meat production has increased more than fivefold since 1950, and factory farming is the fastest growing method of animal production worldwide¹³. This practice continues to grow around the world as a cheap method of producing meat for consumption. However, many of the practices associated with intensive animal farming have been criticized by public health professionals and animal welfare advocates¹⁴. Pork, chicken, and eggs are viewed as important and inexpensive contributors to dietary protein for the expanding urban populations¹⁵. To help dismantle factory farms, humans need to either change their dietary habits or look for alternative sources of protein.

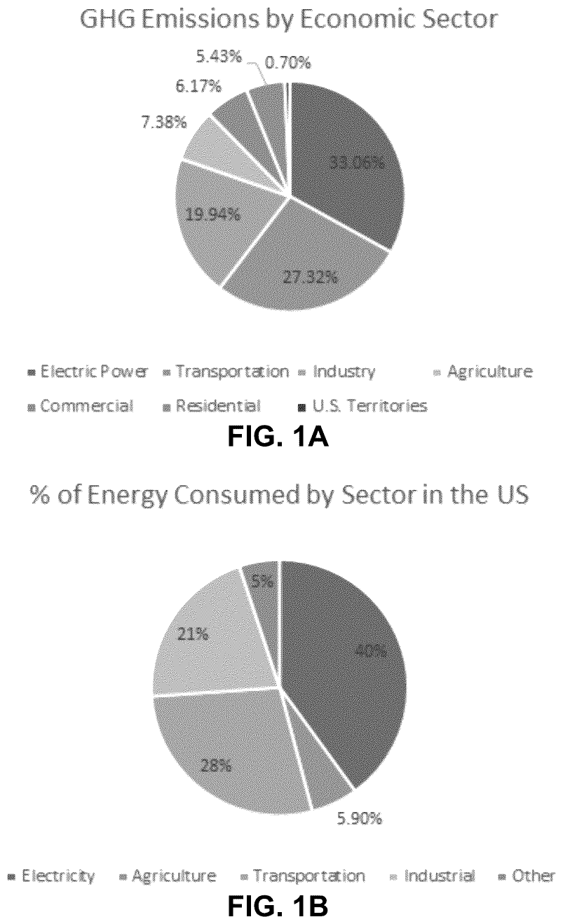

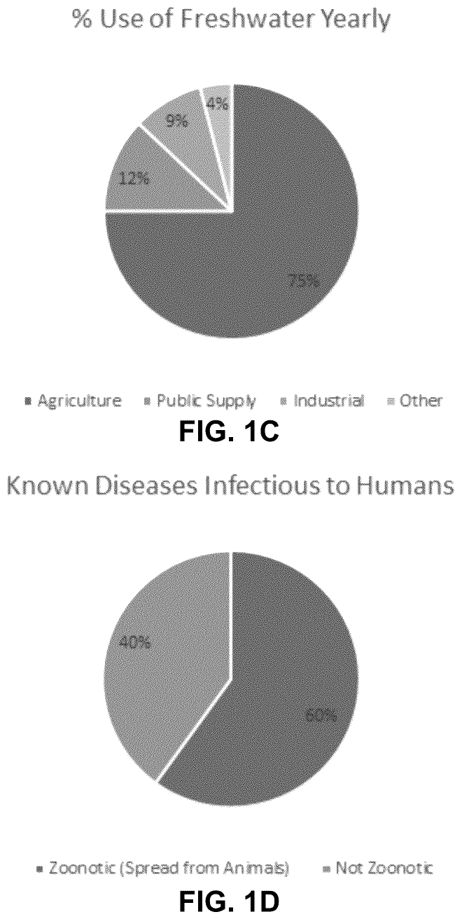

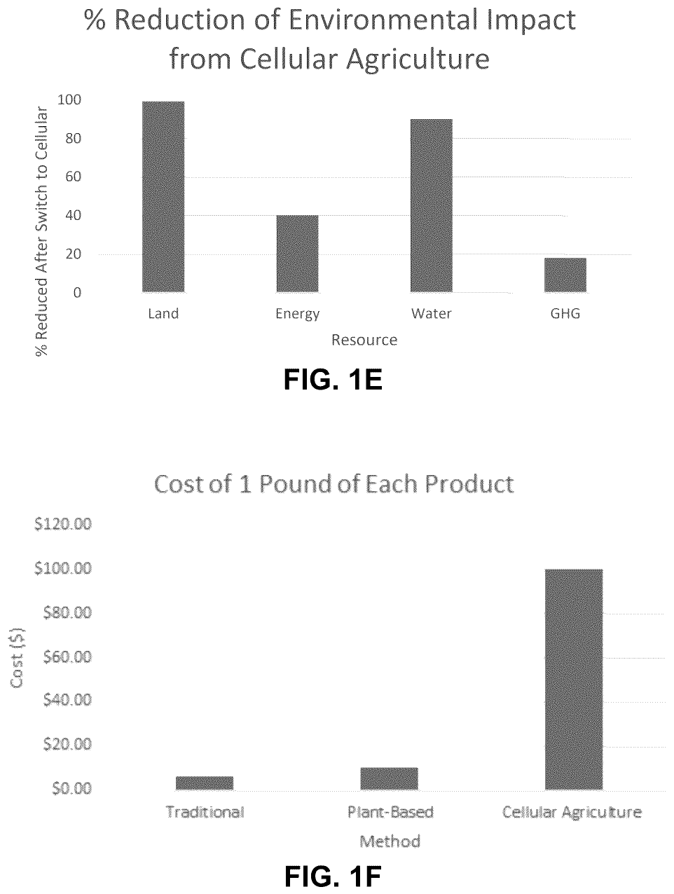

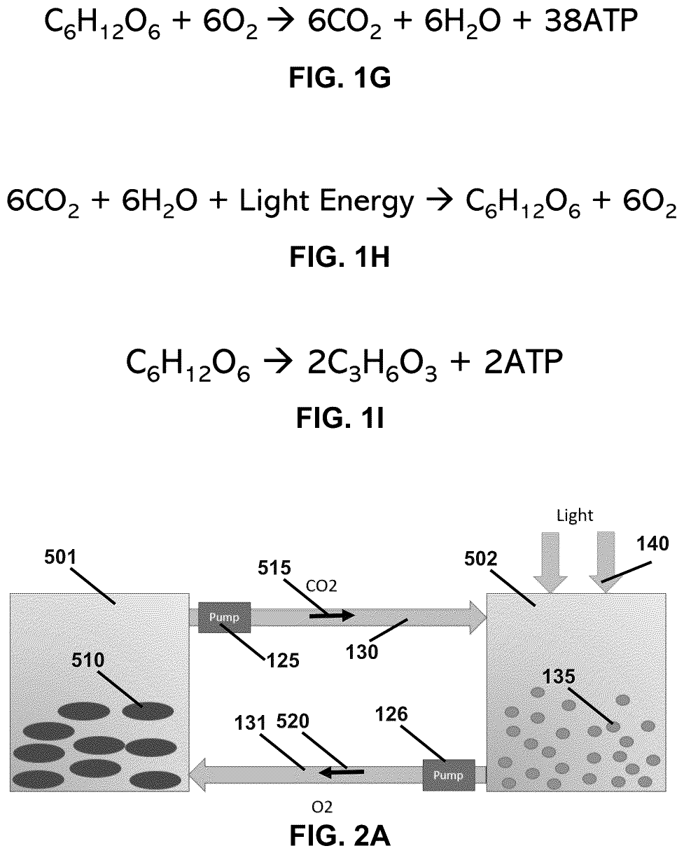

2.1.1 Environmental Impact: Our current farming practices require large amounts of water, energy, and land use, as well as GHG emissions, making factory farming a grave environmental concern⁷. On average, livestock contributes to 9% of total carbon dioxide emissions, 39% of methane emissions, and 65% of nitrous oxide emissions⁷. Although seemingly minor in comparison, agricultural practices account for 7.4 percent of all GHG emissions, as shown in FIG. 1A⁵, which shows a chart of greenhouse gas emissions (GHG) by economic sector. This is still however an egregious number of emissions from agriculture, and all aspects need to be lowered to significantly decrease the impact of GHG emissions.

Humans currently use approximately 50% of total habitable land for agriculture¹⁶. This does not include deserts, the arctic, or other land that is not inhabited by humans on a large scale. Also, as shown in FIG. 1B, 5.9% of energy in the United States is used for agriculture⁶. FIG. 1B⁶ shows a chart of energy consumed by economic sector in the US.

Furthermore, FIG. 1C illustrates that 75% of the total amount of water used by humans is for agriculture¹⁷. FIG. 1C shows a chart of use of freshwater in the US18. To lessen the crisis of our shrinking water supply, industries must research and apply creative solutions to decrease their impact on an increasingly scarce resource.

2.1.2 Health and Safety Concerns: The risk of disease is embedded within modern factory farming¹⁹. Animals in factory farms are treated with enriched feeds and veterinary medicines that stimulate growth but also weaken their immune systems. These animals are also almost genetically identical, and with thousands of them housed close together, the environment is a hotspot for disease incubation¹⁹. As seen in FIG. 1D, the majority (60.3%) of emerging infectious disease events are caused by zoonotic pathogens, which are defined as those which have a non-human animal source²⁰. FIG. 1D shows a chart illustrating a comparison of the origin of diseases infectious to humans²⁰.

2.1.3 Ethical Concerns: There are many ethical concerns with factory farming, from the overcrowding and confinement of animals in small spaces to the removal of beaks or tails from livestock. Veal calves are kept in total darkness for most of their lives and fed a diet of iron-deficient milk substitute to make their flesh palatably pale and tender, to a point just above starvation²¹. All these factors cause a great deal of stress to the animals²¹.

Some laws have been passed to prevent inhumane treatment. California passed the Prevention of Animal Cruelty Act in 2008. This law requires that “calves raised for veal, egg laying hens and pregnant pigs be confined only in ways that allow these animals to lie down, stand up, fully extend their limbs and turn around freely”¹⁴. There is still significant legal headway to be made to ensure all states and countries are treating livestock humanely.

2.2 Cellular Agriculture: Cellular Agriculture is the field that attempts to tackle the issues caused by our current animal agriculture systems. Recent developments in biology and biomedical engineering have led to the ability to grow human tissue in vitro, which created the field of tissue engineering²². These developments include the ability to isolate stem cells and culture them ex vivo⁷. One recent development in the last 15 years is the ability to grow skeletal muscle stem cells, or satellite cells, into full artificial muscle⁷. This new ability paved the way for exploring tissue engineering in the world of animal agriculture²³. If it is possible to grow human muscle in a lab, it should also be possible to grow animal muscle. This was shown in 2013 when the first cultured meat prototype was produced by Mark Post's lab at Maastricht University²³. The process of growing cultured meat starts by obtaining animal muscle tissue²². The tissue is then broken down, and the satellite cells are isolated for proliferation²². After enough satellite cells have proliferated with traditional culture techniques, they are differentiated into mature muscle cells²². This then leads to the final meat product, which in Mark Post's case, was the first cultured beef burger²⁴. Since this demonstration of the potential of cultured meat burgers, this research has spread to more labs around the world, and some small businesses have begun to develop their own product prototypes²⁴.

2.2.1 Solves Contemporary Issues: The appeal of cultured meat is that it has the potential to solve many issues which are caused by traditional meat production methods. Some of the most alluring qualities of cultured meat are that it reduces land, energy, and water used in animal agriculture, and overall GHG emissions.

Current farming practices contribute a large portion of the GHG emissions into the earth's atmosphere. One analysis found that cultured meat has the potential to significantly reduce GHG emissions from meat production⁸. This analysis also found that land usage could be reduced by 99% when compared to beef production⁸. There would also be a 90% reduction in water usage and a 40% reduction in energy usage when compared to beef production⁸. The only category that cultured meat did not improve is the energy consumption when compared to poultry farming⁸. In FIG. 1E, one can see the percentage reduction of each resource if animal agriculture was replaced by cellular agriculture. FIG. 1E shows a chart of potential reduction of environmental impact of meat production by cellular agriculture⁸.

2.2.2 Cost Comparison: Traditional methods of factory farming have been engineered and modified over the years to make the final product as cheap as possible²⁵. The cost comparison between the different meat products, as seen in FIG. 1F, illustrates the massive cost difference between traditional meat culture with cellular agriculture. Traditional meat cost fluctuates depending on where you live, but the average cost is $6.12 per pound²⁶. Plant-based meat is slightly more expensive, at $10.30 per pound²⁷. Comparatively, cell agriculture meat is very expensive and costs around $100 per pound, which currently cannot compete in the general meat market against farmed products²⁷. However, as this field is researched and improved upon, the cost of cellular agriculture meat will only go down²⁴. FIG. 1F shows a chart illustrating the current cost to produce one pound of each meat product^{26,27}.

2.3 Scale-Up Issues: While cultured meat has the potential to improve upon traditional meat in many aspects, this requires the creation of large-scale systems to generate massive quantities to sustain the meat demand. The scale necessary to compete with traditional meat production is said to be the largest ever system for tissue engineering²⁴. Some of the biggest limitations that researchers face for scale-up are related to the cells, suitable scaffolds, cell culture media, and the large-scale bioprocess¹⁹.

For large-scale production of cultured meat products, many limitations are specific to the needs of the cells which are part of the final meat product. One limitation is how long cells can be cultured, as it has been shown that human myoblasts can double up to 45 times for in vitro culture²⁸. This is limited because there is difficulty preventing satellite cells from differentiating⁷. However, there is some evidence that aged populations of satellite cells have similar regenerative abilities when compared to a young standard²⁹. The main two stages for consideration in cell culture are proliferation and differentiation, and there has been much focus in delaying the latter to lengthen the former⁷.

Another goal is to control the muscle cell niche to be more like that of stem cells; this may allow the cells to extend proliferative abilities³⁰. Besides finding ways to improve and control the proliferation and differentiation of satellite cells, there is a focus on the other cell types that make up meat²². Skeletal muscle is not just one type of tissue; it contains muscle, fat, and connective tissues which contribute to the texture, flavor, and nutritional make-up of meat³¹. Taste is an important factor for cultured meat, and researchers are beginning to understand the combination of peptides responsible for these flavors³². However, the lipids in meat cannot be ignored when replicating the taste of traditional meat³³. Overall, the hurdles to overcome for scale-up to commercial products are controlling proliferation, differentiation, and the mixture of cell types.

Scaffolds are an integral part of tissue engineering, as they guide cells in 3D culture and can help promote the growth of functional tissue³⁴. This is especially important for muscle cells since they form tissues with specific orientations³⁴. Therefore, it is necessary to form scaffolds that help to replicate the natural environment of the target cells⁷. One necessary complication to overcome is nutrient transport, which is often addressed by increasing scaffold porosity⁷. Another way to achieve this is using decellularized plant scaffolds, which serve as an effective scaffold for cardiomyocytes³⁵. The benefit of these scaffolds is that they take advantage of the natural vasculature of plants for nutrient transport³⁵. The fabrication of these scaffolds is relatively simple when compared to the synthetic formation of such a vascular network³⁵. Since they are made of natural polymers, these scaffolds are edible³⁵. The idea of an edible scaffold is incredibly important for cultured meat, as the product is intended for consumption³⁶. The scaffold does not have to be edible, but this would mean that cells must be removed before further processing³⁶. Another benefit of having an edible scaffold is that it may contribute positively to the texture of the cultured meat product¹⁹. The type of scaffold used will influence the success of large-scale cultured meat production.

Growing cells require cell culture media to supply nutrients and other necessary biomolecules⁹. Currently, the gold-standard medium for growing mammalian cells uses FBS to supply necessary growth factors and growth inhibitors³⁷. One concern with this method for cultured meat is that the use of FBS conflicts with the advantage of “slaughter-free” meat production⁹. Serum-based media are also flawed because the serum composition varies for each batch, which makes quality control more difficult⁹. Serum-based media is also prohibitive for scale-up because of its cost³⁶. In recent years, the price of FBS in the United States has gone up by 300%³⁸. These kinds of volatile fluctuations in FBS cost are not uncommon and are not conducive to sustainable large-scale operations³⁸. Another concern for cell culture media is the production of waste products. There are some concerns that, like other large-scale operations, waste products may be released into the water supply³⁹. Currently, serum-based media are more effective than completely synthetic media, but the cost and nonstandard nature of FBS are prohibitive for scale-up⁷. However, there is some preliminary evidence that recycling nutrients might improve the capabilities of synthetic media⁸.

2.2.4 Bioreactors: A universal definition of a bioreactor does not exist⁴⁰. There are, however, two main types of bioreactors: chemostat and turbidostat. A chemostat bioreactor includes an ongoing feed of fresh nutrient medium, while also discharging an equal mass of used medium, including biomass. Conversely, a turbidostat bioreactor keeps the biomass constant⁴¹. Bioreactors can operate in batch, bed-batch, and continuous processes⁴². Depending on the type, such as a fixed wall or rotating wall, the bioreactor can be set up for continuous or semi-continuous harvesting.

Bioreactors have many applications in the bioprocessing field, including cell culture and tissue engineering. This ranges from bone, ligaments, blood vessels, and even heart valve tissue. Bioreactors have already improved the processing and the results of skin and cartilage regeneration, the two main laboratory-grown products that are currently commercially available⁴⁰.

Some important considerations for bioreactors are how fast the effluent flow rate is as this can help the system in saving water with a recycle stream if the effluent is fast enough¹⁹. Another consideration when designing a bioreactor is the equipment involved to keep the cells alive and keep operating conditions running smoothly. This includes media storage tanks, heat exchangers, monitoring and control systems, and a means of maintaining isothermal conditions within the bioreactor¹⁹. Each bioreactor must be designed specifically for each tissue because if the signals are inappropriate or absent, cells cannot proliferate or form organized tissues, and instead they become disorganized which leads to cell death⁴³.

2.3 Animal Cell Culture: Cell culture is the process of growing cells in vitro for scientific purposes. The process for cell culturing includes isolation of the cell of interest to culture, growing the cells in media, and passaging the cells as they grow to confluence, or fill an entire container⁴⁴. The skeletal muscle niche has a complex tissue composition with various cell and tissue types that contribute to its quality⁴⁵. Recreating authentic muscle tissue in culture requires many efforts to replicate the structures, texture, and flavors present in traditional meat. Many specifications of culturing conditions must be considered for successful tissue culture.

2.3.1 Culture Requirements: Before considering the chemical components of the media for cell culture, the macroscopic environment for cell growth must be suitable for cell growth by mimicking physiological conditions. Specifically, pH, temperature, and osmotic pressure are conditions that should be monitored and kept consistent. The suitable pH for mammalian cell growth is about 7.2-7.4. CO₂ can have a large effect on the pH of a system because of the formation of carbonic acid. A NaHCO₃—CO₂ buffer system is often utilized to prevent pH from fluctuating⁴⁶. The temperature of the system should be kept at about 37° C. which is consistent with mammalian internal temperature. Oxygen must be kept at high levels to support growth and metabolism as well.

In addition to these essential components for cell growth, considerations should be made to prevent unwanted contamination in the environment. The growth environment must be sterile to prevent the growth of unwanted microorganisms such as bacteria⁴⁷. Sterile techniques must be carefully followed to prevent the incorporation of plasticizers from plastic instruments, trace elements from water, or microorganisms⁴⁸. To prevent contamination and thus product spoiling, antibiotics, and antimycotics are often included in cell culture media⁴⁶. Specifically, penicillin and streptomycin may be utilized in media. It should be noted that these are not essential to growth, and depending on the purpose of the product, antibiotic use may not be preferred by potential consumers since antibiotic-fed farm animals have been a larger concern in the past decade²³.

Satellite cells also need a physical scaffold or structure on which to develop in culture to generate a 3D structure similar to muscle tissue. The scaffold must allow for nutrient flow, proper anchorage, and generate a viable product⁴⁵. A tissue cell culture environment may be simulated with biomaterials such as hydrogels or macroporous structures, which may even be edible materials such as decellularized plants⁴⁵.

The necessary nutrients required for cell culture include sugars, inorganic salts, pH buffers, amino acids as a nitrogen source, vitamins, fats, nucleic acid precursors, growth factors, hormones, antibiotics, and O₂⁴⁴. Each cell type has a unique set of nutrients in its ideal media which must be determined experimentally. Often found in cell culture media, which is artificial, or not derived directly from animal tissue, is serum. Serum provides many essential nutrients such as salts, various growth factors, and essential proteins for cell growth, however, there are serum-free media available for cell culture⁴⁷.

Growth factors present in media designate conditions either of the two main growth pathways of satellite cells. The two major pathways for cells when in culture are proliferation and differentiation. For proliferating cells, key growth factors must be present at different cell cycle growth determining steps. Fibroblast growth factor (FGF), specifically FGF2, is an essential growth factor involved in signaling pathways to initiate continuous satellite cell proliferation⁴⁹. In vivo, FGF is released when skeletal muscle cells are injured to induce satellite cell proliferation for tissue regeneration⁵⁰. FGF also blocks the differentiation of satellite cells, thus reinforcing proliferation pathways⁵¹. In a medium dedicated to satellite cell proliferation, there must be a source of growth factors which is typically serum⁵². Serum-rich media support the proliferation of cells, whereas serum-poor media are needed for cell differentiation.

Albumin is an essential molecule to preserve the integrity of the grown satellite cells. Bovine serum albumin (BSA) is a common component of media for cell culture. BSA in serum is typically 60% of the total protein in serum at a concentration of about 50 mg/mL⁵³. There are many positive effects associated with albumin. Albumin plays a role in reducing oxidative stress, acting as an antioxidant to aid in circulation in vivo, which is also essential in vitro⁵⁴. Albumin also has been suggested to play a role in preventing apoptosis by modifying forces in the extracellular environment of large bioreactors⁵⁵. With the ability to bind a variety of ligands of vastly different chemical makeup, including fatty acids, metal ions, and amino acids, albumin can influence many cellular processes including waste removal⁵⁶.

Amino acids must be in high concentrations in tissue culture media. Amino acids are the nitrogen source for satellite cells and are essential for tissue growth and protein synthesis. Specifically, glutamine is essential at a higher concentration than other amino acids in mammalian cell culture⁴⁶.

Other notable nutrients include salts, hormones, and micronutrients. One salt of note is sodium bicarbonate which regulates pH by buffering CO₂ concentrations. Insulin may also be added to media for its hormone activity in regulating and promoting the use of glucose and amino acids⁴⁶. Various inorganic salts, amino acids, vitamins, and other compounds are common in formulations of media such as DMEM basal medium⁴⁴.

2.3.2 Limiting Factors: The health of animal cell cultures can decline due to the accumulation of toxic metabolic byproducts. The primary by-products of concern are ammonium and lactic acid, which can limit cell function and proliferation^{57,58}. Ammonium is the main nitrogenous waste product of mammalian cells and is secreted as a waste product of protein synthesis. Lactic acid is a carbon based compound produced by anaerobic respiration. During aerobic respiration, the carbon byproduct is carbon dioxide. When maintaining cell cultures, these waste products are removed by replenishing the culture with fresh cell culture media.

2.4 Co-cultures-2.4.1 Benefits: Co-culture of animal cells with other species is useful in many different contexts. Coculture systems can be used to help better mimic in vivo tissue models by creating a more accurate cellular environment. Other than studying the natural interactions between the cell 11 cultures, researchers can also use it to better understand ways to improve the cultivation success of cell lines⁵⁹⁻⁶¹. Researchers have been using co-culture systems to help better understand the microenvironment in cancer research⁶². It is also widely used by scientists to help determine the potential cytotoxicity of drug compounds due to a more accurate in vivo environment⁶⁰. Furthermore, in the field of synthetic biology, the study of various co-culture systems can also help create new synthetic interactions between different cell populations⁵⁹.

In the realm of synthetic biology, researchers have been studying different ways to employ a co-culture of different organisms to achieve various metabolic end-products^{63,64}. Before this discovery, researchers have been using a single type of engineered microbe to help aid in the conversion of substrate to the desired product. However, research has shown that using a combination of organisms in a “divide and conquer” technique can improve the efficiency of the biosynthesis of desired compounds⁶³. In addition to a “divide and conquer” technique, studies have also revealed that one can manipulate and modulate the production of compounds through co-cultures⁶⁴.

2.4.2 Carbon Cycle: Carbon must be present in usable sources for animal cell metabolism. The most common carbon sources in media are sugars, specifically in monosaccharide form, most commonly as glucose for aerobic respiration⁴⁶. In commonly used media for optimal mammalian cell culture, DMEM, glucose is present at 4500 mg/L concentration⁶⁵. Carbon sugar sources are transformed into metabolic chemical compounds in the cells that generate chemical energy in the form of carrier molecules such as ATP, NADH, and FADH2. Energy from these carrier molecules is used in the cell for various metabolic processes including protein synthesis, DNA synthesis, waste removal, vesicle trafficking, among others. During aerobic respiration, which requires O2 as a reactant, the metabolic byproduct is carbon dioxide, as seen in FIG. 1G⁶⁶, which shows the overall equation of aerobic respiration.

Carbon dioxide in the atmosphere is recycled via photosynthesis by plants and other photosynthetic organisms by using water as a hydrogen source to ultimately produce sugars⁶⁷. This process is depicted in FIG. 1H, which shows the overall equation of photosynthesis. If oxygen is not present, the animal cells undergo lactic acid fermentation, as seen in FIG. 1I⁶⁶, which shows the overall equation of lactic acid fermentation. In FIG. 1I, the by-product, lactic acid, is written as C₃H₆O₃.

2.4.3 Nitrogen Cycle: The nitrogen cycle is a biogeochemical process where inert nitrogen found in the atmosphere is transformed into more usable forms for various organisms. The nitrogen cycle can be characterized as five interconnected stages: nitrogen fixation, nitrification, assimilation, ammonification, and denitrification. Nitrogen fixation consists of converting inert nitrogen, found in the atmosphere, into a more usable form, ammonia. Nitrification is when ammonia is converted into nitrite and subsequently into nitrates. Assimilation is the process where plants uptake the various nitrogen compounds used in the formation of amino acids and proteins. Assimilation is the process that allows nitrogen to enter the food web. Ammonification is when various bacteria and fungi convert organic matter containing nitrogen back into ammonium when an organism dies. Denitrification is the process where nitrogen is released back into the atmosphere by converting nitrates into nitrogen gas⁶⁸.

Nitrogen metabolism is an aspect of the nitrogen cycle that plays an important role in the survival of many living organisms⁶⁹. Nitrogen metabolism is the process in which an organism can uptake and recycle ammonia, or its charged form, ammonium, and convert it to various compounds including proteins, nucleic acids, hormones, neurotransmitters, and nucleoside triphosphates^{69,70}. Various studies have suggested that the manipulation of this mechanism in algae and cyanobacteria can be used to recycle built-up ammonium in animal cell cultures⁷¹⁻⁷³.

2.4.4 Potential Cell Types-2.4.4.1 Plant Cells: Plant cells are a potential cell type to consider for their low cost, easily replicated growing conditions, and photosynthetic capabilities. Plant cells for culture are cheap and easy to isolate and general methods for harvesting metabolically active and photosynthetic cells are well established⁷⁴. Developing a plant cell culture includes isolation of tissue from the explant, culturing of the callus, and generation of a cell culture suspension from the callus in media⁷⁵. The growth condition for plant cell culture generally requires a temperature of 24-25° C. with photoperiodic light⁷⁵. The short life cycle and easy manipulation of their small genome make plant cells ideal candidates for research purposes⁷⁶. Additionally, due to plant cells being photosynthetic, they require few nutrients besides oxygen, water, and ample light to thrive. An additional benefit to the photosynthetic abilities of plants in co-culture is the uptake of carbon dioxide generated by cellular respiration.

Of the many possible plant species to use, Arabidopsis thaliana is a well-characterized species that can grow successfully in suspension culture⁷⁷. Studies of A. thaliana demonstrate the simple techniques to generate large cell suspensions from callus cultures⁷⁸. Other cell types to consider for a co-culture environment would be those with high capacity for cell growth, high viability with high cell density, and efficient metabolism for utilizing waste products of animal cell metabolism.

2.4.4.2 Microalgae: The application of algae to help remove waste prevents another possibility. For example, a mixed culture of microalgae and bacteria can remove ammonium and phosphate from domestic sewage and convert them into various valuable compounds such as algal metabolites and biogas⁷². Research has also suggested the potential benefits of using algae to recycle waste metabolites produced in mammalian cell culture¹¹. The microalga, Chlorococcum littorale, can increase the proliferation of both C2C12 (mouse myoblast) and rat cardiac cells through co-culture¹¹. Co-culture of C. littorale with mammalian muscle cells can decrease glucose consumption and lactic acid production, increase O2 concentrations by metabolizing carbon dioxide, and significantly reduce the ammonium buildup in media¹¹. This co-culture also reduced the cardiomyocyte production of creatinine kinase, which is an enzyme that indicates muscle cell damage^{11,79}. This indicates that co-cultures of microalgae can increase the health of muscle cells. The microalga Chlamydomonas reinhardtii is also able to uptake ammonium as a nitrogen source⁸⁰⁻⁸². C. reinhardtii is a model organism for algae⁸², and it has been used in co-culture with fibroblasts to reduce hypoxic distress in low-oxygen conditions⁸³. Chlorella vulgaris is another microalga species that can uptake ammonium from cell culture media^{84,85}. Both C. reinhardtii and C. vulgaris are edible and contain essential amino acids, fatty acids, and minerals⁸⁶.

2.4.4.3 Cyanobacteria: Cyanobacteria are photosynthetic prokaryotes that are thought to be one of the earliest organisms to have lived on this planet. Aside from being known for their photosynthetic abilities, studies have shown that they can also be used to treat distillery wastewater^{71,87}. Their ability to fix nitrogen and their photoautotrophic nature make them cheap to maintain⁷¹. Their ability to produce reactive oxygen species serves to break down recalcitrant contaminants that are usually non-biodegradable⁷¹. In addition, cyanobacteria are also known for up taking metal ions and phosphates⁷¹. There has not been much research on cyanobacteria and animal cell co-cultures because certain metabolites that cyanobacteria secrete are toxic to many organisms⁸⁸. However, research has shown that manipulating culture conditions of cyanobacteria, like temperature, can reduce toxin secretion⁸⁹.

Additional research has suggested that cyanobacteria hydrolysate can also be used as a nutrient supplement for muscle cell growth⁸. Culturing meat using cyanobacteria hydrolysate, when compared with traditional beef production, resulted in a 45% reduction in energy use, 96% reduction in GHG emissions, 99% lower land usage, and 96% reduction in water usage⁸. This demonstrates that cyanobacteria can potentially produce nutritional components for muscle cells. However, minimal research has been done on whether cyanobacteria can secrete these metabolites for muscle cell growth.

2.4.5 Genetic Engineering Possibilities: The use of genetic engineering to help increase the production of various compounds is a relatively new field of biology that researchers have been exploring. Various transgenic bacterial species are used to synthesize compounds such as insulin, biofuels, and enzymes⁹⁰. However, genetically engineered bacteria can harbor harmful side effects and be a potential health hazard⁹¹. Therefore, the usage of genetically engineered bacteria has been mainly limited to pure production purposes. The potential use of genetically engineered plants or algae to produce desired metabolites has also been an area of interest for many. Genetically engineered plants and algae can produce many more complex proteins, due to their increased cellular complexity compared with bacteria. Many bacteria lack the proper post-translational modifiers to 14 process and modify more complex proteins, which makes plants and algae more promising candidates for producing complex proteins⁹².

### 3. Project Strategy

3.1 Initial Client Statement: Our inventive team aimed to alleviate some of the limitations of large-scale cultured meat production. The initial background research showed that the cost of media in large-scale production was prohibitive and raised environmental concerns. With this research, and input from scientists in the field of cellular agriculture, this initial client statement was developed: Recycle the spent cell culture media to be reused for future cell culture.

3.2 Technical Design Requirement: The team found that recycling the media required the design of a novel system. There has been minimal research on the feasibility of a media recycling system using symbiotic cocultures. However, no significant work has been made on the development of a prototype or proof-of-concept. The team determined multiple design objectives that would satisfy the need for this system. Various cell culture constraints were determined to ensure that the co-culture system is feasible. These objectives and constraints informed the design process and experimental setup.

3.2.1 Objectives: The team determined objectives that, if achieved, would fulfill the need for this coculture system. To meet the need, the system must be scalable, allow proliferation and harvesting of PBSCs, and reduce the cost associated with cell culture media.

3.2.1.1 Scalable System: Scalability is necessary for this system to improve the large-scale production of cultured meat so it can compete with traditional meat production. To create a scalable system, the cells must be grown in a bioreactor, which can range from small-scale to industrial-sized. There must be a scaffold for the PBSCs to grow on since they are adherent cells. The system must transport the spent media to be recycled and transport the replenished media back to the PBSC culture.

3.2.1.2 Proliferate PBSCs: Proliferation is the step in cultured meat production when a large mass of cells is produced, which will be used to form muscle tissue. The PBSCs must continue to proliferate in the system so a large biomass can be produced. The media must contain components that encourage PBSC proliferation, like FGF2.

3.2.1.3 Harvest PBSCs: After proliferation, the cells must differentiate into more specialized tissues to form a meat product. This system must allow easy harvesting of PBSCs when they are ready to move to the differentiation stage. This objective may be accomplished by physically separating the PBSC culture from the symbiotic recycling culture.

3.2.1.4 Reduce Media Cost: The system must reduce media-associated costs. This can be achieved by increasing the lifetime of the cell culture media by 100%. This will reduce the frequency of media 16 replacements, thus reducing the cost associated with media consumption. The media lifetime can be extended by removing metabolic waste products like ammonium, lactic acid, and carbon dioxide. The system should also add oxygen back into the media.

3.2.2 Constraints: The team determined cell culture constraints that would be necessary to adhere to for the system to be feasible. For the system to work, the PBSCs must remain viable and retain their stem-phenotype, and the co-culture cell must remain viable and metabolically functional.

3.2.2.1 PBSC Viability: The PBSCs must remain viable in the co-culture system. The co-culture cell and the coculture conditions must not significantly hinder the growth of PBSCs. The PBSCs must be viable after proliferation to differentiate into muscle tissue.

3.2.2.2 Maintenance of PBSC Stem Phenotype: PBSCs can proliferate well because they are stem cells. After differentiation, the proliferative ability of the cells diminishes. To produce a large cell mass, the co-culture system must encourage PBSCs to maintain their stem-phenotype.

3.2.2.3 Co-culture Cell Viability: For the co-culture to function, the co-culture cell must remain viable throughout the proliferation process. This means that the co-culture cell must be able to survive in similar culture conditions to the PBSCs.

3.2.2.4 Co-culture Cell Functionality: Along with remaining viable, the co-culture cell must maintain its ability to metabolize animal cell waste products. The co-culture conditions must not inhibit the uptake of waste products or the production of vital nutrients.

3.3 Standard Design Requirements: For the design of this project, there are standards of regulation that must be followed. Since the goal of this bioreactor system includes food production, both the FDA and USDA standards of meat production must be followed as it relates to the quality and safety of meat production for consumption. There are also design regulations for the bioreactor set forth by Good Manufacturing Practices and International Organization for Standards. Both regulations cover safety, cleanliness, and general good operations for this bioreactor to avoid accidents or contamination.

3.3.1 Bioreactor Standards: For the bioreactor itself, many standards must be complied with to consider it valid. The bioreactor must be sterile and easy to be cleaned, along with safe to work with for employees. Through the design process, the following regulations must be met: ISO 22000-Food safety management systems-Requirements for any organization in the food chain⁹³; ISO 11737-2—Sterilization of health care products—Microbiological methods—Part 2: Tests of sterility performed in the definition, validation, and maintenance of a sterilization process⁹⁴; and ISO 31000-Risk management⁹⁵.

ISO 22000 shows companies what they need to do to demonstrate how they can control food safety hazards. Mostly just a guideline on how to safely handle and store food. ISO 11737-2 is a guide to properly sanitize equipment. ISO 31000 lays a guideline for organizations facing any risks, no matter the size of the organization. It is just a general approach to solving some common risks or problems.

In addition to the ISO regulations, there are Current Good Manufacturing Practices (e.g., GMP) guidelines set by the FDA to set the standards for pharmaceutical products. Although our bioreactor isn't strictly for pharmaceutical use, this is still a product obtained from cell culture and falls under these guidelines. The main CGMP standard we will follow is: CGMP Part 210—Current Good Manufacturing Practice in Manufacturing, Processing, Packing, or Holding of Drugs⁹⁶.

This regulation is to ensure the product meets quality design requirements and the whole assembly process is followed safely. Failure to comply with these regulations, “shall render such drug to be adulterated under section 501(a)(2)(B) of the act and such drug, as well as the person who is responsible for the failure to comply, shall be subject to regulatory action”⁹⁶.

3.3.2 Standards for Meat Production: For the meat production standards, the group will follow the regulations set by the USDA. The role of the USDA is to regulate packaging, handling, and labeling. Many of the current USDA meat standards relate to livestock so we can disregard a good amount as they include living conditions or ways to correctly handle the animals. Several of the relevant meat production standards are shown here: 315 Pathogen Intervention—The harvest process shall include at least two pathogen intervention steps. One of the intervention steps shall be a critical control point (CCP) in the establishment's FSIS recognized harvest process Hazard Analysis Critical Control Point (HACCP) plan. The CCP intervention(s) shall be scientifically validated to achieve a three-log reduction of enteric pathogens⁹⁷. 318.2 Handling-All boneless beef shall be maintained in excellent condition⁹⁷. 318.7 Microbial Testing-All lots of fresh chilled boneless beef shall be tested for all indicator microorganisms⁹⁷. 331 Fat Percentage—The limits for percent fat in a boneless beef product should be within 12.0-18.0%⁹⁷.

Standard 315 covers the intervention of pathogens, which is the methods the production company uses to prevent diseases in their meat products. Of course, the best methods are preventative methods, but this is up to the company on how to handle this. Standard 318.2 is just to make sure the boneless beef is stored correctly. Standard 318.7 says to test all boneless beef 18 for microorganisms, which is to make sure correct storage guides are followed and protect consumers from disease. The final standard, 331, covers the fat percentage of boneless beef products, which is relevant to cellular agriculture.

It should be noted that the FDA does not regulate meat production for most farm animals. It does, however, require all food manufacturers except seafood and juice processors to create a Food Safety Plan. This plan has the owner evaluate the hazards that could affect food manufactured, processed, packed, or held by such a facility. It also wants them to identify and implement preventive controls to significantly minimize or prevent the occurrence of such hazards and provide assurances that such food is not adulterated or mislabeled. For example, 21 U.S.C. § 350 g-Hazard analysis and risk-based preventive controls⁹⁸ is applicable.

3.4 Revised Client Statement: Addressing the prohibitive cost of media in large-scale cultured meat production is necessary to compete with the traditional meat market. The requirements previously explained must be met to address this limitation. With these requirements in mind, the team formed the following revised client statement: “Develop a scalable system to recycle spent media so it can be used again for PBSC culture. The system must keep PBSCs viable and promote proliferation. The PBSCs should be harvestable from the system for relocation to the differentiation stage. The system must reduce media-associated costs by extending the media lifetime by 100%. This can be achieved by removing metabolic waste products and replenishing vital nutrients. The cells responsible for media recycling must remain viable and functional in the co-culture system”.

3.5 Project Approach: The inventive team began with background research on the need for a co-culture media recycling system. The team also brainstormed conceptual designs for a bioreactor to house the co-culture. By the end of A term (late October), the team chose to use C. reinhardtii as the co-culture cell and began to learn techniques for growing PBSCs. The team spent B term (late October through early December) starting preliminary experiments and learning proper techniques for culturing PBSCs and C. reinhardtii. The preliminary experiments explored the growth of PBSCs and C. reinhardtii in various media conditions and temperatures. The team also began experiments to assess the ability of C. reinhardtii to uptake waste metabolites and extend the media lifetime. Over winter break, the team began co-culturing PBSCs with C. reinhardtii. The team spent C term (late January through mid-March) exploring the effects of the co-culture on dissolved oxygen and pH. The effects of growing the co-culture with a semi-permeable membrane were also explored. In D term (mid-March through early May), the team finished all validation experiments. The data was analyzed, and the report was finalized. The team participated in Project Presentation Day with a recorded presentation and a live Q&A session.

### 4. Design Process

The following will outline the needs analysis for the two main aspects of the system, a description of various conceptual designs, and the selection of a final design using a custom value analysis matrix.

4.1 Needs Analysis: The needs were addressed for two aspects of this design. The first is the co-culture system, which is largely defined by the specific cell species used to co-culture with PBSCs. The second is the scale-up, which is concerned with bioreactor designs that can incorporate the coculture system for large-scale cultured meat production.

4.1.1 Co-culture System: The team brainstormed needs for the co-culture system, and each need was assigned a weight based on relative importance in Table 1. The weight ranged from 1 to 5, with 1 being not very important and 5 being very important.

TABLE 1
NEEDS AND RELATIVE WEIGHTS
FOR THE CO-CULTURE SYSTEM.

| Need | Weight |
|---|---|
| Metabolize CO₂ | 5 |
| Uptake Ammonium | 5 |
| Uptake Lactic Acid | 5 |
| Overlapping Temp. Requirement | 4 |
| Overlapping pH Requirement | 4 |
| Well Documented Species | 3 |
| Transfectability | 2 |

The most important needs are metabolizing carbon dioxide into O₂, uptaking ammonium, and uptaking lactic acid. These are the primary metabolic waste products in animal cell culture. Removing them from the media is the first step in media recycling.

The next most important needs are having an overlap in temperature and pH requirements for the two cell types. Normal culture conditions for PBSCs are 37° C. and a pH of 7.2. PBSCs can be grown at a lower temperature, but their metabolic rate will be slowed. The cell type used in co-culture should be able to survive well in similar conditions to the PBSCs.

The next need in order of importance is having a cell type for co-culture that is well documented. It is advantageous to work with a species that is well studied, so more time can be spent on experimental design instead of learning to culture the cells.

The least important need is transfectability. The team discussed the possibility of creating a transgenic cell that produces growth factors that promote PBSC proliferation. This may become a more important objective following the success of toxic byproduct removal.

4.1.2 Scale-Up: For scale-up of the co-culture system, the inventive team brainstormed multiple needs and gave them relative weights, as seen in Table 2.

TABLE 2
NEEDS AND RELATIVE
WEIGHTS FOR SCALE-UP.

| Need | Weight |
|---|---|
| PBSC Harvestable | 5 |
| Scalability | 5 |
| Keep Co-culture Separate | 4 |
| Suitable Scaffolding | 4 |
| Variable Temperature Control | 3 |
| Durability/Ease of Maintenance | 3 |

The harvestability of primary PBSCs was deemed to be the most important because this is the whole idea behind culturing the cells in a bioreactor. If the cells cannot be harvested, then there is no point in scaling up the process. Scalability of the design is also very important, as that pertains to taking the small-scale model and recreating an industry-scale version for marketing and sale.

Next separating the co-culture and suitable scaffolding for the cells were decided to be given a weight of four. Although these were not the most important factors in the scale-up of our design, they are very integral and important to the success of the scale-up. Separating the coculture is important in our design to avoid contamination between the algae and PBSCs. Suitable scaffolding for the cells pertains to the holding system for the cell interactions, which is important to ensure the proliferation of the PBSCs.

Finally, ease of maintenance and variable temperature control were both given a weight of three. Ease of maintenance is how easy the equipment can be cleaned after use or how often material needs to be changed to avoid breaking or damage. Variable temperature control is keeping the cells in viable living conditions.

4.2 Conceptual Designs: Multiple designs were brainstormed as solutions to the media-use issue in large-scale cellular agriculture. For the basic co-culture system, multiple cell species were explored to find a suitable co-culture cell. For scale-up, multiple conceptual designs were created for bioreactor systems.

4.2.1 Co-Culture System: The main component of the co-culture system is the species of co-culture cell that is being co-cultured with PBSCs. Inspiration was drawn from the carbon and nitrogen cycles in our ecosystem, so multiple photosynthetic cell species were explored.

4.2.1.1 Chlamydomonas reinhardtii: The unicellular microalga C. reinhardtii was explored as an option for the co-culture cell in the co-culture system. C. reinhardtii is a model species for algae, so its culture techniques and conditions are documented⁹⁹. The microalga is photosynthetic, motile, and phototropic. C. reinhardtii can uptake ammonium, which is a necessary step in media recycling⁹⁹⁻¹⁰¹. The optimal temperature for culturing the microalga is 28° C., but it has been grown from 18° C. to 37° C.¹⁰². C. reinhardtii is often grown in TAP media, which can have a pH of 6.9-8.8103. Transfection of C. reinhardtii has been successful by electroporation¹⁰⁴ and CRISPR¹⁰⁵.

4.2.1.2 Arthrospira platensis: Arthrospira platensis is a cyanobacterium that was explored as a cell species for the coculture system. A. platensis is an edible and photosynthetic bacterium that is sold as a dietary supplement, more commonly referred to as spirulina¹⁰⁶. The cyanobacterium can uptake ammonium in culture¹⁰⁷. The optimal temperature for growth is 29° C., and it has been grown at a range of 23° C. to 34° C.¹⁰⁸. A. platensis is grown at an optimal pH of 9.5108. Transfection of A. platensis has been successful by electroporation¹⁰⁹ and using agrobacteria¹⁰⁶.

4.2.1.3 Arabidopsis thaliana: Arabidopsis thaliana is a model organism for plants that was explored as a source of cells for the co-culture system. This is the only multicellular organism proposed, but it is well characterized, and its cells can be isolated and cultured. There is some evidence of A. thaliana uptaking ammonium, but this process is downregulated as ammonium is taken into the roots and then the cytoplasm¹¹⁰. The optimal culture temperature for A. thaliana cells is 24° C., and the culture pH is 5.5111. Transformation of A. platensis has been demonstrated at the organismal and cellular level using agrobacteria¹¹² and tymovirus¹¹³ transfection.

4.2.2 Scale-Up: To be able to scale up the design the group must develop a system to contain the cells that will allow the nutrient exchange but keep them separate to avoid contamination. Several concepts of this design were created and are shown below.

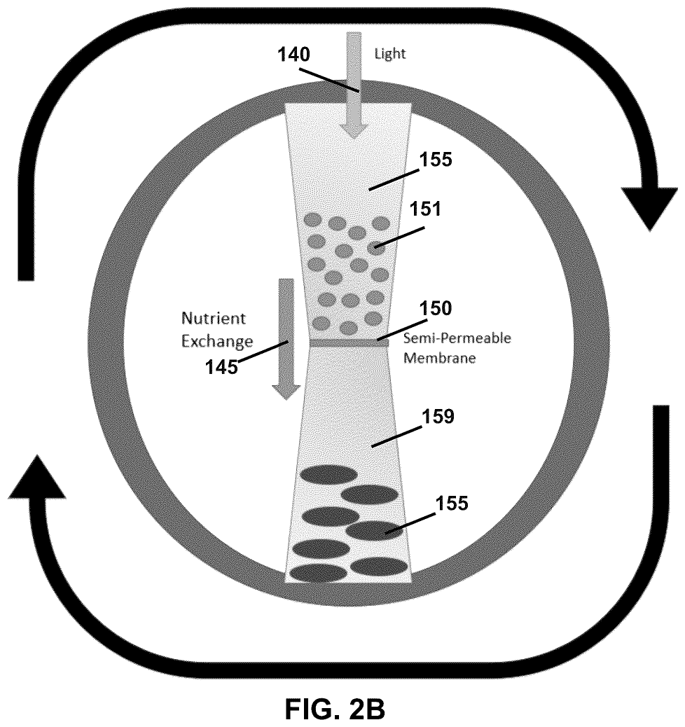

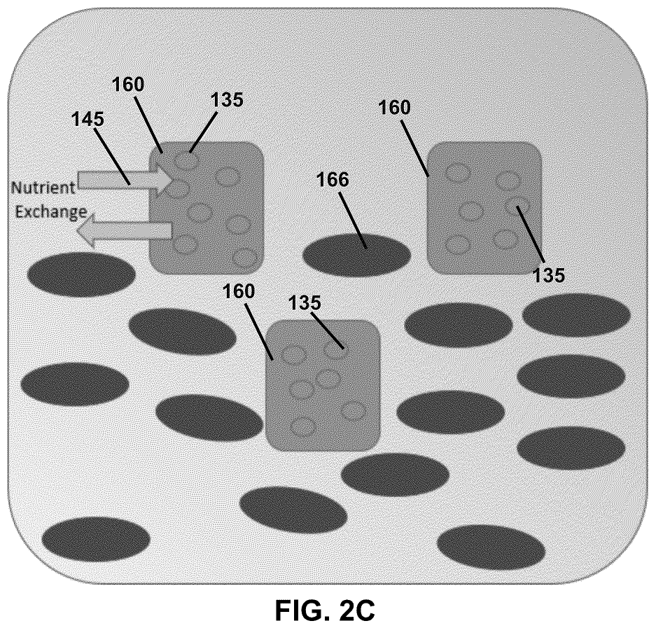

4.2.2.1 Simple Pump: The first design the group considered was keeping both cells separate with two vats connected with pumps, as illustrated in FIG. 2A, which shows an initial design of a simple pump according to some aspects of the technology. This design, at its core, is likely the simplest and easiest to design and build due to its simplicity. The main drawback to this design is its maintenance with many parts that require cleaning such as the membranes and pumps. FIG. 2A shows a diagrammatic representation of a simple pump design according to some embodiments. In FIG. 2A, the live bovine cells 510 are illustrated in mammalian culture reactor 501 living and producing waste metabolites 515 that is pumped at waste pump 125 through waste conduit 130 and into symbiotic reactor 502 wherein light 140 is shining upon algae cells 135 that consume and/or dispose of waste metabolites 515 and produce beneficial nutrients 520 that arrive at nutrient return pump 126. The nutrient return pump 126 pumps the beneficial nutrients 520 through nutrient conduit 131 and into mammalian culture reactor 501, wherein the live bovine cells 510 thrive upon beneficial nutrients 520.

4.2.2.2 Hourglass: The second design considered was an hourglass design that replaces the two pumps with a gravity-fed transfer system. A semipermeable membrane separates the two containers in the center, which allows the byproducts to pass through. The hourglass will also be on a motorized spinning wheel, to avoid the buildup of waste on one side and allow even transfer. This design is a little out of the box and would hopefully be little maintenance, but the biggest issue is how the PBSCs would be harvested. FIG. 2B shows a diagrammatic representation of the hourglass design according to some aspects of the technology. In FIG. 2B, light 140 enters symbiotic culture chamber 155 wherein symbiotic cells 151 consume waste that enter the symbiotic culture chamber 155 through a nutrient exchange 145 that can occur through the semi-permeable membrane 150 from product cell reactor 159 that contains product producing cells 155.

4.2.2.3 Hollow Fiber: Hollow Fiber bioreactors are used in cell culture making them a prime option for this project. The idea is to pump the algae cells in DMEM around the bioreactor, where the algae cells enter a cartridge. This cartridge allows the flow of algae to separate into many different capillary tubes, with PBSCs on the outside of these tubes. A semipermeable membrane separates the two cells which will keep them from contaminating each other but allow the exchange of the carbon dioxide and O2. This design concept is well established and has been studied extensively.

4.2.2.4 Teabag: This design is unique in that the cells are grown in the same vat and mostly stay there together. FIG. 2C shows a diagrammatic representation of the teabag design. The algae cells 135 would sit inside a semipermeable membrane packet 160, like a teabag, and would allow the nutrient exchange 145 into the PBSCs 166 surrounding the packets. This idea is like how tea diffuses from leaves into water to make tea, which inspired the name.

4.3 Final Design Selection: To choose a final design, a custom value analysis matrix was used to compare each conceptual design. This considered the needs and weights stated in Section 4.1 above and how well each design satisfied the needs.

4.3.1 Co-culture System: To choose a cell species for co-culture with PBSCs, a value analysis matrix was used to compare the proposed co-culture cells. Each species was rated on a scale from 1 to 5 based on how well it satisfied each objective as shown in the value analysis matrix of Table 3. The satisfaction score was multiplied by the weight, and each of these values was added together for a total score. The highest score indicates the design that best fulfilled the needs, taking relative importance into account.

TABLE 3
VALUE ANALYSIS MATRIX FOR EVALUATING
THE CO-CULTURE SYSTEM.

| | | Chlamydomonas | | Arthrospira | | Arabidopsis | |
|---|---|---|---|---|---|---|---|
| | | reinhardtii | | platensis | | thaliana | |
| Need | Weight | Satisfaction | Total | Satisfaction | Total | Satisfaction | Total |
| Metabolize | 5 | 5 | 25 | 5 | 25 | 5 | 25 |
| CO₂ | | | | | | | |
| Uptake | 5 | 4 | 20 | 4 | 20 | 3 | 15 |
| Ammonium | | | | | | | |
| Uptake Lactic | 5 | 0 | 0 | 0 | 0 | 0 | 0 |
| Acid | | | | | | | |
| Overlap of | 4 | 3 | 12 | 3 | 12 | 2 | 8 |
| Temp. | | | | | | | |
| Requirement | | | | | | | |
| Overlap of | 4 | 5 | 20 | 3 | 12 | 2 | 8 |
| pH | | | | | | | |
| Requirement | | | | | | | |
| Well | 3 | 5 | 15 | 4 | 12 | 5 | 15 |
| Documented | | | | | | | |
| Transfectable | 2 | 4 | 8 | 4 | 8 | 4 | 8 |
| Total | | | 100 | | 89 | | 79 |

Based on the results of the value analysis matrix in Table 3, Chlamydomonas reinhardtii best fulfills the design needs for the co-culture system. Arthrospira platensis was promising, but the high pH requirement was its most detrimental factor. Arabidopsis thaliana also had issues with pH, as well as temperature, and its ability to uptake ammonium is not very promising. None of the proposed cell species could uptake lactic acid, which suggests that a second cell species may be necessary for further development of the co-culture system.

4.3.2 Scale-Up: Shown in Table 4 is an analysis of the group's several scale-up designs. Based on the scale-up objectives laid out above in Table 2, each design was scaled on how much it satisfies each objective and given a score according to the importance of that objective.

TABLE 4
VALUE ANALYSIS MATRIX FOR EVALUATING THE SCALE-UP DESIGN.

| | | Simple Pump | | Hourglass | | Hollow Fiber | | Teabag | |
|---|---|---|---|---|---|---|---|---|---|
| Need | Weight | Satisfaction | Total | Satisfaction | Total | Satisfaction | Total | Satisfaction | Total |
| PBSC | 5 | 4 | 20 | 4 | 20 | 5 | 25 | 4 | 20 |
| Harvestable | | | | | | | | | |
| Durability | 3 | 3 | 9 | 3 | 9 | 5 | 15 | 4 | 12 |
| Co-culture | 4 | 5 | 20 | 4 | 16 | 5 | 20 | 4 | 16 |
| Separate | | | | | | | | | |
| Temp | 3 | 5 | 15 | 2 | 6 | 3 | 9 | 2 | 6 |
| Control | | | | | | | | | |
| Scaffold | 4 | 4 | 16 | 3 | 12 | 5 | 20 | 4 | 16 |
| Scalability | 5 | 5 | 25 | 2 | 10 | 5 | 25 | 4 | 20 |
| Total | | | 105 | | 73 | | 114 | | 90 |

Based on the totals given in Table 4, the hollow fiber bioreactor design satisfies the objectives the most. The simple pump design is very close to the hollow fiber design, due to its simplicity and ease of scalability, but the hollow fiber is a more solid and chosen design.

### 5. One Final Design Verification

### 5.1 Standard Laboratory Procedures:

5.1.1 PBSC Feeding: PBSCs were regularly maintained according to the volume of the culture flask. For T-25 flasks of PBSCs, after aspirating media, cells were fed with 5 mL of DMEM+10% FBS, 1% penicillin/streptomycin, and growth factors. For T-75 flasks of PBSCs, after aspirating media, cells were fed with 11 mL of DMEM+10% FBS, 1% penicillin/streptomycin, and growth factors. Cells were fed one day after passaging, and every 2-3 days beyond the initial feeding until the next passage. See Example 1.

5.1.2 PBSC Passaging: The purpose of PBSC passaging is to either increase the cell population for experimentation or to prevent PBSC contact inhibition. PBSCs were passaged once the cells reached around 70% confluency. The PBSC passaging protocol is detailed in Example 1.

5.1.3 PBSC Thawing: Frozen PBSCs were removed from liquid nitrogen and immediately thawed according to the detailed protocol in Example 1. The cells were taken from the same cow sample for each thaw, except for the final reader experiment, results from these cells are shown in Section 5.3.3 below.

5.1.4 C. reinhardtii Maintenance: C. reinhardtii maintenance was carried out to ensure a healthy population of microalgae for experimentation. The C. reinhardtii culture was maintained on a shaker in an incubator at 27° C. with a 12-hour light cycle. The health of the algae was examined by the coloration of each flask, with deep green indicating a healthy culture with minimal death and high proliferation, and a bright yellow indicating more cell death. See Example 2 for more information on algae culture maintenance.

5.1.5 Transwell Experiments: The Transwell experiments conducted to verify the feasibility of algae and PBSC coculture utilized 6-well plates with Transwell inserts (VWR, Corning). The protocols used for these experiments are included in Example 4 below.

5.1.6 Oxygen Evolution with Hill Reaction: The hill reaction is an oxidation reaction that quantifies the amount of a colorimetric indicator, DCPIP, which is oxidized by a sample. The goal of the hill reaction for our purposes was to quantify exactly how much oxygen is evolved by the algae in different media conditions and at different concentrations. This would determine the concentration of algae needed to fulfill the oxygen requirements of the PBSCs, without oversaturating PBSC cultures with oxygen. Multiple different protocols were developed, however there were no conclusive results from the concentrations of algae per volume studied. The hill reaction results were no longer deemed necessary when new equipment was obtained by the lab which provided direct readings of 28 dissolved oxygen and pH in any cell culture flask. The protocol which achieved inconclusive results is included in Example 3.

5.1.7 Staining: Hoechst stains were conducted on PBSCs to help with cell counting and imaging upon conclusion of co-culture experiments with algae to help define the two cell types from each other. The Hoechst staining protocol is detailed in Example 5.

A live/dead stain was used to identify the viability of PBSCs after co-culture with algae cells for three days. Live/dead stains were conducted on PBSCs for cell imaging of viable cells using Calcein AM and Ethidium homodimer-1 (Invitrogen) according to protocols available from the product manufacturer. See Example 6 for protocol details.

5.1.8 ID Reader Set-up: Readers and biosensors were supplied from Scientific Bioprocessing (SBI). The two readers we used for data collection required setup and calibration per the manufacturer instructions which are present in Example 7. Biosensors for both dissolved oxygen (DO) and pH were placed in experimental flasks according to manufacturer instructions (Example 7).

5.2 Determining Characteristics of Monoculture—5.2.1 PBSC and C. reinhardtii viability and growth in various media compositions: Before growing the two cell types of interest together and assessing their growth in a coculture environment, it was important to identify whether each cell type could survive in conditions that met the needs of both cell types. In other words, we needed to determine the ideal culture conditions which would proliferate PBSCs without differentiation, and in which algae cells could also survive and undergo photosynthesis.

Initial experiments were conducted to compare the growth of PBSCs in DMEM, the ideal growth medium of PBSCs, and in TAP, the ideal growth medium of C. reinhardtii. Passage 4 PBSCs were plated at 10,000 cells per well in a 6-well plate and various ratios of DMEM and TAP were used as the media. The cells were grown at 37° C., with 20% O2, and 5% carbon dioxide. Unfortunately, all trials were contaminated due to an issue with serum contamination and results were inconclusive although they suggested that PBSCs could not survive in higher ratios of TAP, since no nutritional needs were met by the contents of TAP medium for PBSC growth. It was clear that DMEM should be the growth medium if our objective were to proliferate PBSCs.

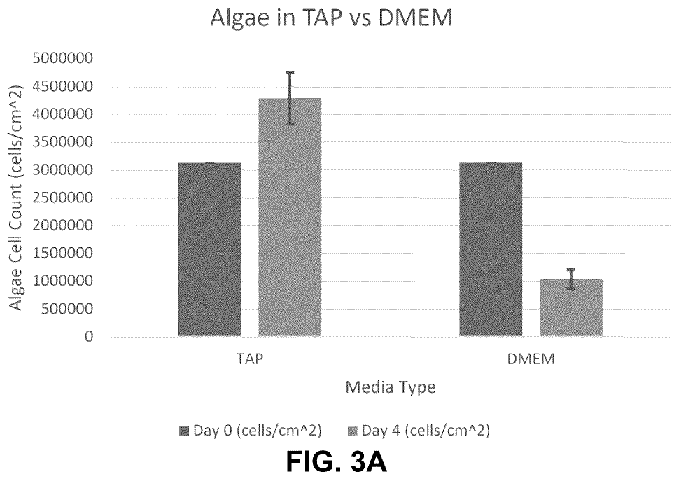

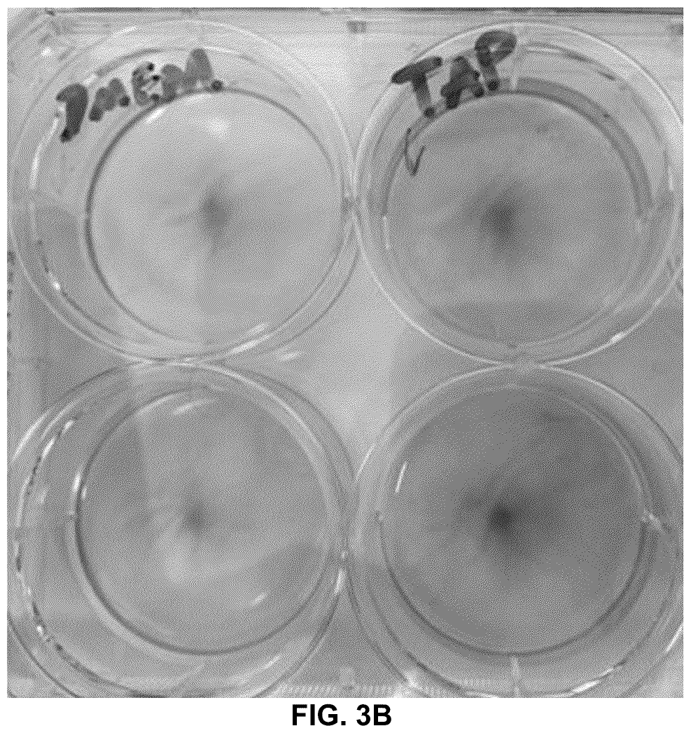

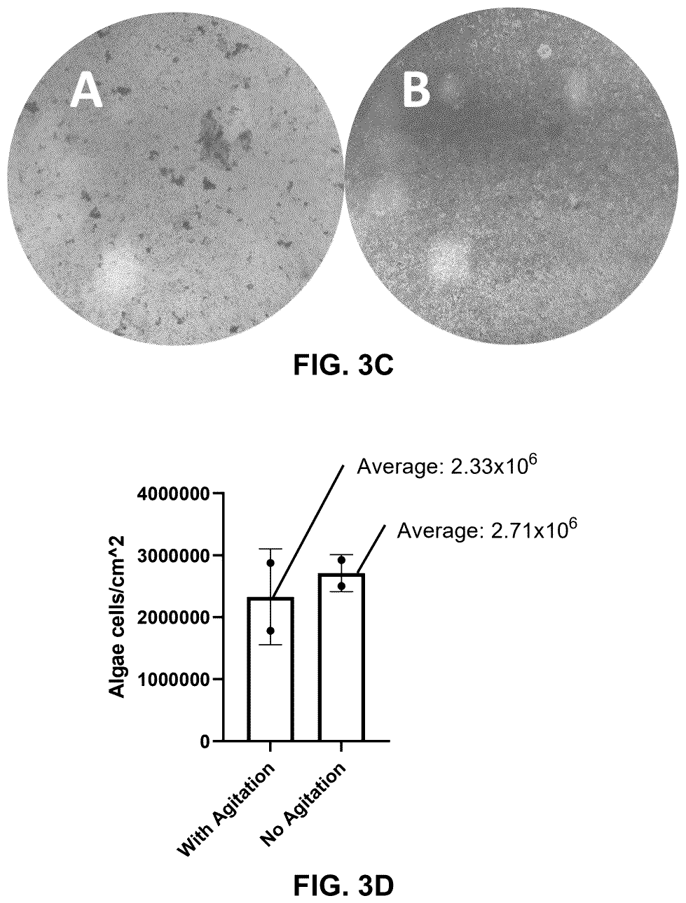

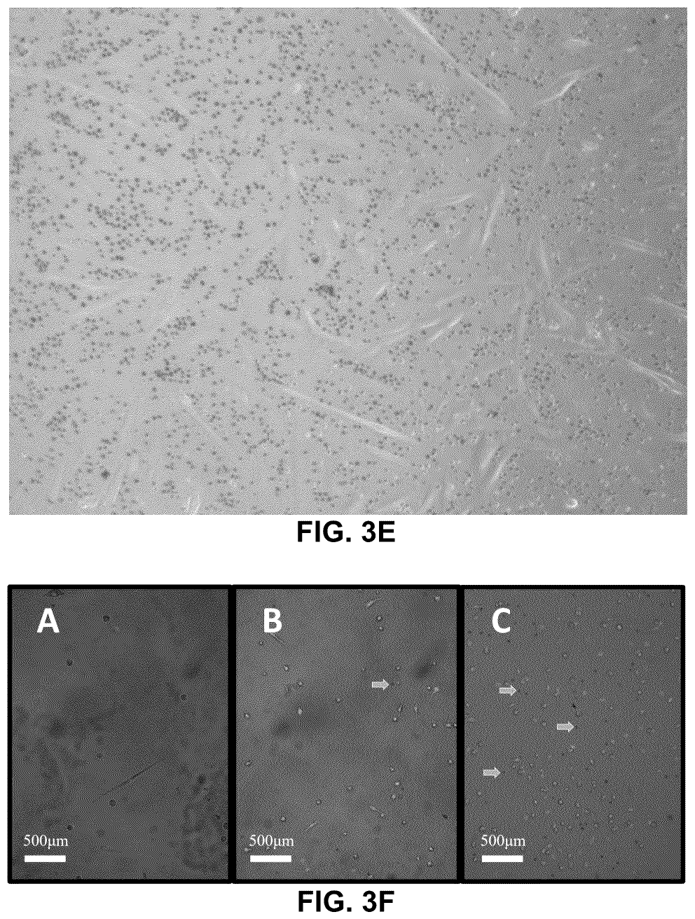

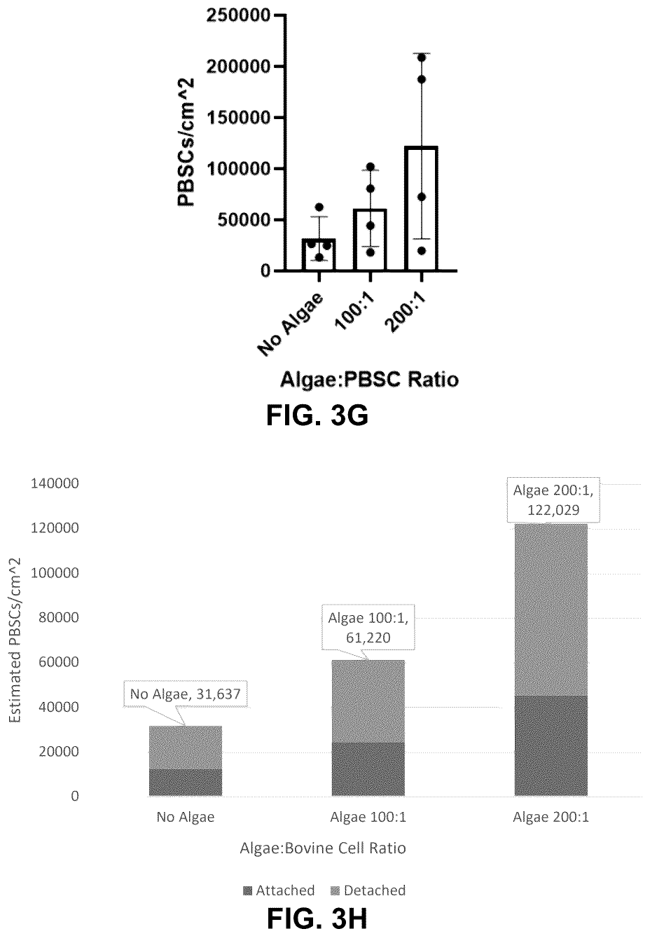

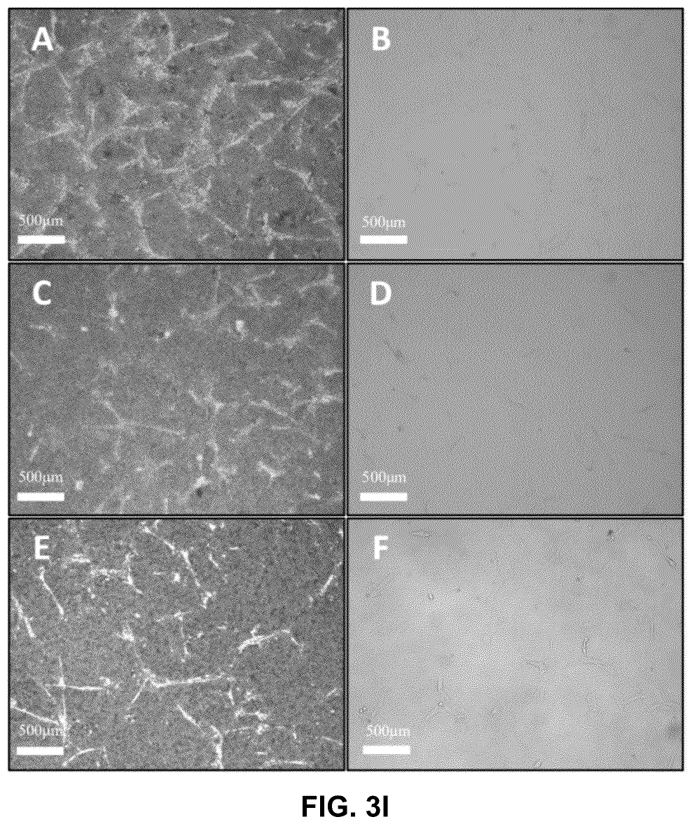

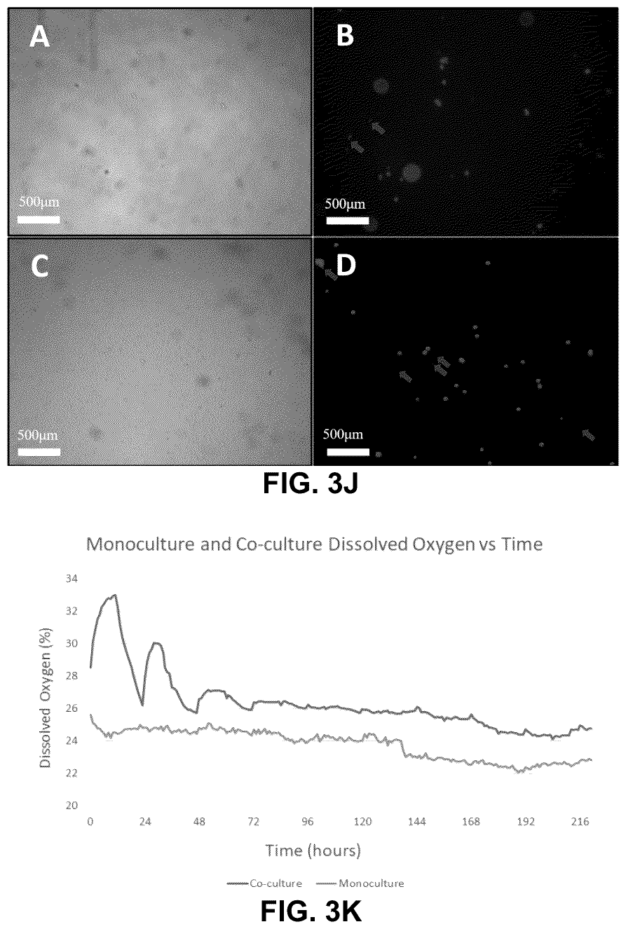

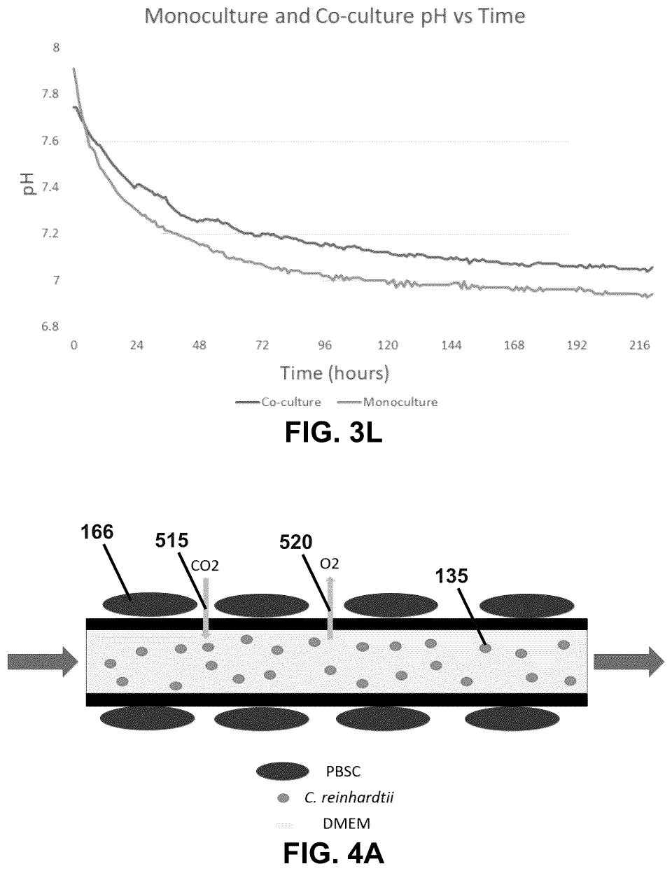

Similar to the goal of the previous experiment, the next experiment aimed to test the viability of C. reinhardtii in DMEM compared to TAP. The algae were plated in a 6-well plate at 10,000,000 cells per 3 mL well at 30° C., with 5% O2 and 5% carbon dioxide in a 12-hour on/12-hour off light cycle. Two of the wells had DMEM media and the other two wells had TAP media. The initial cell count was recorded, and the final cell count was recorded after four days of culturing. The results showed that the algae population increased significantly in TAP from about 3 million cells/mL to nearly 14 million cells/mL and remained at about the same population size when in DMEM, specifically decreasing by about 33,000 cells/mL (FIG. 3A).

FIG. 3A shows a comparison of C. reinhardtii growth in TAP media and DMEM media after four days of culture. As seen in the bar chart of FIG. 3A, C. reinhardtii was unable to exhibit any significant growth in the DMEM media in four days, however, upon examination of the algal cells, it was determined that the algae remained viable despite not being able to proliferate. The viability of the algae was assessed based on the color, movement, and “clumpiness” as dead algae forms clumps in floating media. FIG. 3B shows an image of the two treatments of TAP and DMEM.

In FIG. 3B, the algae media comparison in DMEM and TAP Image was taken after 2 days in at 30° C., with 5% O2 and 5% carbon dioxide in a 12-hour on/12-hour off light cycle. The left column shows algae in DMEM, while the right shows algae in TAP.

5.2.2 Experiment with Algae Agitation: Once it was determined that C. reinhardtii was able to remain viable in DMEM, our group wanted to investigate whether the algae required constant agitation for successful proliferation. This would determine whether we needed to culture algae separately from PBSCs since PBSCs needed consistent, undisturbed conditions to properly adhere to the plate surface and remain viable. The stock cultures were known to proliferate best in Erlenmeyer flasks on shaker platforms at larger volumes of 50 mL or more. The angled sides of Erlenmeyer flasks ensure equal distribution of nutrients on a shaker platform. In this experiment, C. reinhardtii was cultured in a 30° C. incubator in TAP, 5% 02, 5% carbon dioxide, with 500,000 algae initially plated on a 6-well plate. The experiment was done with 2 replicates with one biological sample. The 6-well plate design was not as conducive to algae cell growth but was conducive to PBSC growth since 6-well plates are tissue-culture treated and provide a consistent flat surface to which cells can adhere. The sides of the 6-well plate are shallow and thus placing the plate on a shaker would potentially shake media out of the plate. Thus, the small magnetic stir bars provided agitation without the risk of losing media from the plate. Half of the 6-well plate contained stir bars, and the other half did not contain stir bars as the control as seen in FIG. 3C.

FIG. 3C shows at left, (A), C. reinhardtii culture after 48 hours with constant stir bar agitation and at right, (B), C. reinhardtii culture after 48 hours without any stir bar agitation.

FIG. 3D shows a graph of cells counts for the algae agitation experiment (n=2). After determining the ideal media conditions and whether algae required agitation for proper cell growth, we aimed to evaluate if PBSCs could tolerate the light conditions needed for algae survival and photosynthesis. Literature suggested that light conditions would not be specifically detrimental to PBSCs, although regular physiological conditions do not provide the cells with light¹¹⁴. Additionally, the optimal temperature of the co-culture would have to be experimentally determined in the next experiments to facilitate both algae and PBSC growth.

Our next tests aimed to synthesize the findings from the separate growth cultures and apply the newfound knowledge about ideal culture conditions to an algae-PBSC co-culture.

### 5.3 Creating a Co-Culture System

5.3.1 First co-culture experiment: In the first co-culture experiment, the PBSCs and C. reinhardtii were seeded in a T-75 flask in DMEM with no IGF at 37° C., with 20% O₂ with no growth lights. The aim of this experiment was to investigate whether the cell types could be cultured together without physical separation as well as the temperature requirements for the co-culture system. Literary research has suggested that C. reinhardtii will not function normally at 37° C. due to heat stress and that 30° C. the maximum temperature that still allows C. reinhardtii to be metabolically viable. Our group demonstrated the effects of heat stress on algae experimentally. FIG. 3E shows the algae with a darker brown color, which is indicative of heat stress or a lack of light^{11,114}.

In FIG. 3E, the image of the co-culture on Day 5 was at 20× magnification. Upon examination of the PBSCs, it was determined that the cells did not appear to exhibit any differentiation or stress responses as a result of direct co-culture with algae cells. This experiment was not held in hypoxic conditions, so it cannot be concluded that algae contributed significantly to the successful proliferation of PBSCs. From this crude example of the effects of co-culture of algae and PBSCs, the group needed to further characterize the effects of algae on PBSCs at a molecular level and determine if there were more ideal conditions that would support the growth of algae in addition to PBSCs. Certainly, moving forward, the growth conditions needed to be more amenable to the needs of algae, specifically with temperature and light conditions.

5.3.2 Transwell Experiments: The goal of the experiment was to compare the growth rates and viability of PBSCs in co-culture, in hypoxic conditions, and with various concentrations of algae. This would determine the ideal concentration of algae needed to fully support the oxygen requirements of PBSCs and adequately recycle carbon dioxide produced by PBSCs. This would also determine if there were any toxic effects on PBSCs with higher or lower concentrations of algae. From previous experimentation with algae and PBSCs, we determined that a ratio between 1 to 50 PBSCs to algae cells and 1 to 200 algae cells provided significant oxygen to the system. Additionally, it was determined that the PBSCs would grow best on the tissue-treated plate rather than the top well insert of the Transwell. This experiment tested the effect of no algae, a 1:100 ratio of PBSCs to algae, and a 1:200 ratio of PBSCs to algae. The cells were plated in a 6-well Transwell plate with the algae in the top well insert and the PBSCs in the well of the plate at 50,000 PBSCs per well. The total algae plated in the 1:100 ratio wells was 5,000,000 algae cells, and the total algae plated in the 1:200 ratio wells was 10,000,000 algae cells.

After three days, the well inserts containing the algae were removed. It was noted that some algae cells were small enough to fit through the filter of the Transwell insert and entered the culture directly with the PBSCs. This is noted by the light (or green) arrows in FIG. 3F. The lower wells containing the PBSCs only were stained with Hoechst stain to identify individual cells. The samples were fluorescently imaged with a 4× objective, and the images shown in FIG. 3F. A-C are representative of an 8× magnified image. There were four images taken from each well to count and identify PBSC concentrations. Representative images are shown in FIG. 3F. Example 9 below includes the calculation of the PBSC cell count from the images taken on the fluorescent microscope and its quantification can be seen in Table 5.

FIG. 3F shows representative images (A-C) from the Transwell Hoechst stain (Left to right: no algae, Algae 100:1, Algae 200:1). Blue fluorescence represents the stain of DNA in PBSCs. Green (or light) arrows point to examples of algae cells in the images within panels B and C.

Table 5 is a quantification of the average number of PBSCs in the fluorescent microscopy pictures for the varying PBSC-C. reinhardtii ratios.

TABLE 5
AVERAGE NUMBER OF PBSCs IN VARYING PBSC-
C. REINHARDTII RATIO WITH STANDARD DEVIATION

| PBSC: | Total PBSCs/ | Attached PBSCs/ | Detached PBSCs/ |
|---|---|---|---|
| Algae Ratio | cm\hat{ }2 | cm\hat{ }2 | cm\hat{ }2 |
| 1:100 | 61220 ± 32297 | 24242 ± 12960 | 36979 ± 20145 |
| 1:200 | 122029 ± 78616 | 45196 ± 28239 | 76833 ± 50687 |
| 1:0 (control) | 31637 ± 18498 | 12326 ± 9187 | 19311 ± 10418 |

The data gathered in Table 5 was compiled through an experiment with two samples for each ratio with two pictures per well. The data obtained from the pictures illustrated that the 1:200 ratio contained the highest number of PBSCs. In all three ratios, there seemed to be more detached PBSCs than attached PBSCs. However, since no live-dead staining was made, no conclusion can be drawn regarding the viability of the PBSCs in the co-culture system. From the Table, our group was able to determine the size of each of the images and thus estimate the total number of PBSCs in a well after three days of co-culture as seen in FIG. 3G.

FIG. 3G shows the estimated number of PBSCs in a Transwell after three days of co-culture at varying ratios. The cell counts in FIG. 3H are representative of four counts taken from different images of different locations in each well plate (See Example 8 for discussion of other images). The PBSCs appeared to proliferate at higher rates in the 1:200 PBSC to algae ratio. FIG. 3H illustrates the estimated number of PBSCs per well in the three different ratios. Compared to the initial seeding density of 50,000 cells, the hypoxic conditions in DMEM without algae caused cell death since the final cell count after 3 days was only 30,380 cells. In the 1:100 PBSC to algae ratio, the final cell count was 58,787 suggesting a minor increase in proliferation but a lack of significant cell population growth. With the 1:200 PBSC to algae ratio, the cell count was 117,180 cells which are more than double the initial seeding concentration of PBSCs. This suggests that the increase in the concentration of algae cells in relation to PBSCs has a positive impact on the outcome of PBSC viability.

FIG. 3H shows estimated counts for PBSCs with different Algae: PBSC ratio, including attached and detached cell counts (n=4). Total PBSCs counted per condition, including both attached and detached, are labeled at the top of each bar in FIG. 3H.

5.3.3 Percent Dissolved Oxygen and pH Reader Experiments: In this experiment, we aimed to again determine the viability of the PBSCs in a direct coculture system by performing live/dead staining. The direct co-culture was conducted in a T-25 flask in the same conditions as the Transwell experiment from 5.3.2, however, this experiment tested the effects of direct interactions between algae and PBSCs indirect co-culture. This would demonstrate the ability of algae cells to grow in direct contact with PBSCs to assess if there were any issues with contact inhibition for either cell type. The viability stain at the end of the three days of co-culture would determine if there were significant impacts of co-culturing PBSCs with algae compared to PBSCs growing without algae in monoculture. Due to the limitation on the number of readers, the sample size of each reader experiment was one. However, the experiment was conducted three times to ensure a detailed analysis.

FIG. 3I shows co-culture with PBSCs and algae cells at (A) Day 1, (C) Day 2, (E) Day 3, and monoculture at (B) Day 1, (D) Day 2, (F) Day 3. All photos are shown at 20× magnification. There was an issue with the live/dead staining of the cells which caused the PBSCs to detach from the culture flask. The cause of this is unknown, however, the stain still showed that cells were viable despite detachment from the flask. The images in FIG. 3I were taken on Day 1-3 of the experiment. The algae cells grew well in direct contact with the PBSCs, and the images of the co-culture show that the PBSCs have proper morphology indicative of proliferation. The live dead staining determined that the viability, regardless of whether cells were detached or attached, was at least 90% in the 200:1 algae ratio (FIG. 3J).

FIG. 3J shows Live/Dead stain of monoculture (A, B) and co-culture (C, D) at 8×. Brightfields are shown in A and C of the respective fields of view images in B and D. Panels B and D show the viable cells in green, and dead cells in red. Arrows point to dead cells in red (or visible arrows). To determine the effects of C. reinhardtii in a co-culture system with PBSCs, our group has decided to quantify the percentage dissolved oxygen (DO) and the pH over a 10-day period. In this experiment, both the co-culture and the monoculture control were cultured with a 12-hour light cycle at 30° C. under hypoxic conditions with a PBSC seeding density of 50,000 cells per well. The ratio of PBSCs to C. reinhardtii in the co-culture system was 1:200 as determined in the earlier experiments to be the most optimal ratio. The comparison of the percentage change DO concentration and pH can be seen in FIG. 3K and FIG. 3L, respectively.

FIG. 3K shows a comparison of the % dissolved oxygen in the monoculture and co-culture system over a 10-day period. As seen in FIG. 3K, the co-culture exhibited increased DO concentrations when compared with the monoculture's DO throughout the 10-day incubation period. This suggests that the presence of C. reinhardtii in the co-culture system has resulted in an increase in the percentage of dissolved oxygen in the system through photosynthesis. It should also be recognized that after three days, the algal cells did not seem to be as productive in generating oxygen for the system. This is an important finding to note when considering the scale-up of the bioreactor on whether the C. reinhardtii should be replenished with nutrients or replaced after three days to maximize efficiency.

FIG. 3L shows a comparison of the pH in the monoculture and co-culture system over a 10-day period. FIG. 3L illustrates a comparison of the pH values of the co-culture with the control over a 10-day period. The data in FIG. 3L suggests that the co-culture was able to dampen the decrease in pH over the 10-day culture period. There are multiple possible explanations for this observed phenomenon. Our team hypothesized that the gradual decrease in pH could be attributed to uptake of carbon dioxide through photosynthesis, affecting the sodium bicarbonate buffer in the DMEM media. Another possible explanation could be a decrease in lactic acid production due to an increase in aerobic respiration from an increase in the DO concentration.

The data obtained from the two other reader experiments were deemed to be unpresentable due to human and random errors. In the first reader experiment, the graph had multiple asymptote-like spikes because the flasks were removed from the readers periodically for imaging, resulting in a slight misalignment between the sensor and the readers. The results obtained from this experiment can be seen in Example 10 below. The data gathered from the other experiment was incomplete due to multiple unexpected shutdowns of the computer. The group decided that the random fragments of the data were not representative of the experimental results and therefore excluded from the analysis.

### 6. A Final Design Validation

6.1 A Final Design: One final design had two main aspects. The first is the co-culture system, which comprises the cell types, culture medium, temperature, and other culture conditions that will be discussed in further detail. The second is the scale-up, which is the proposed bioreactor design for large-scale production using our co-culture system. Both aspects will be discussed further in the following sections.

6.1.1 Co-culture System: The co-culture system uses the microalga C. reinhardtii to uptake metabolic byproducts of PBSC culture and replenish the media with oxygen. The system is closed to gas exchange, so all additional oxygen is supplied by C. reinhardtii. The co-culture is seeded at a 200:1 ratio of C. reinhardtii to PBSCs in DMEM supplemented with 10% FBS, 1×P/S, and growth factors (see Example 11 for full media making protocol). The cells are grown at 30° C., which slows down the metabolic activity of PBSCs but allows C. reinhardtii to function at an ideal temperature. The co-culture is grown with a 12-hour light cycle to allow C. reinhardtii to undergo photosynthesis while allowing the PBSCs to grow for 12 hours in their ideal conditions.

6.1.2 Scale-up: The scale-up of the co-culture system will involve the use of a hollow fiber bioreactor. Hollow fiber bioreactors are widely used for cell culture and have lots of different models and size versions, which is perfect for the plan of scale-up of our system. The group did several instances of modeling and calculations for the bioreactor, as discussed below.

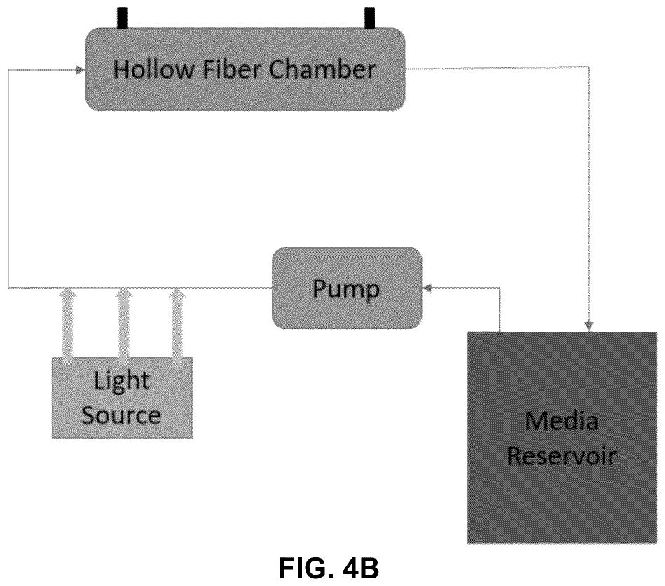

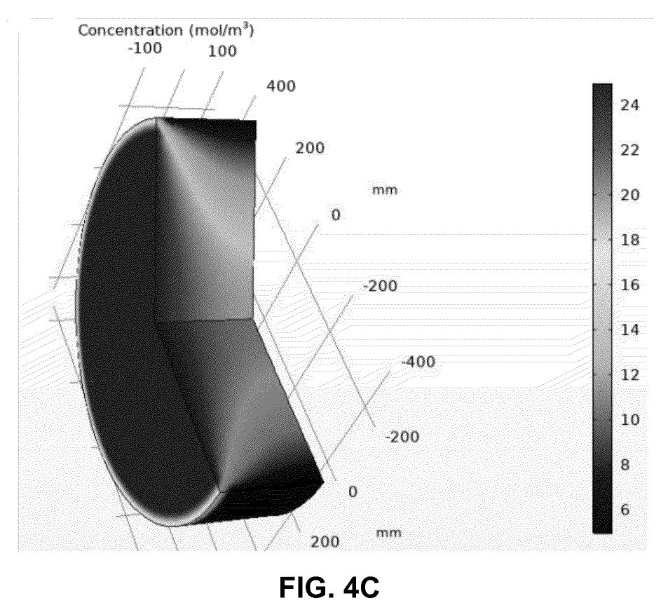

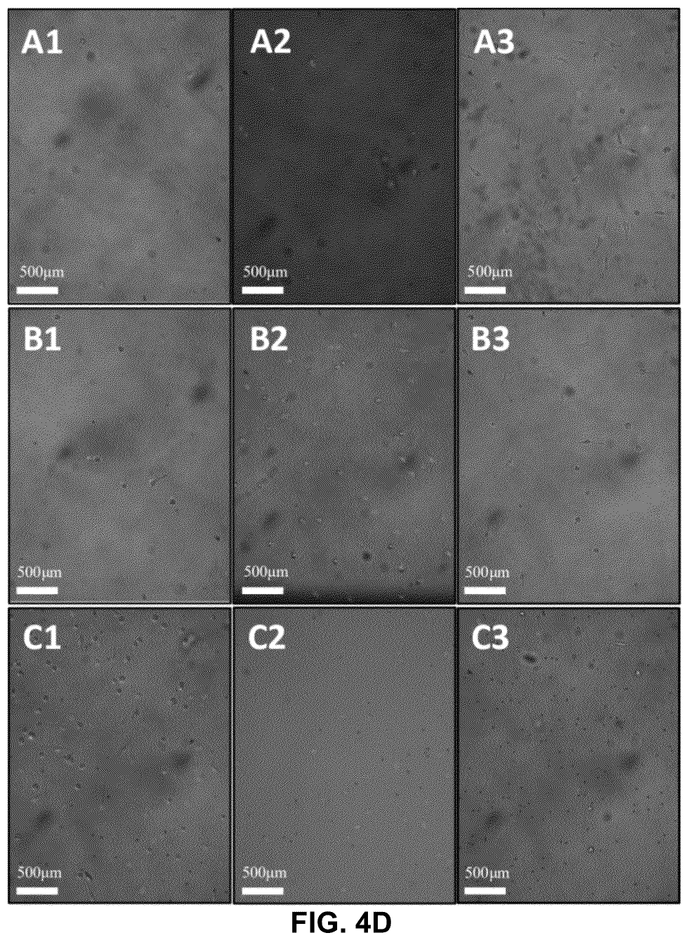

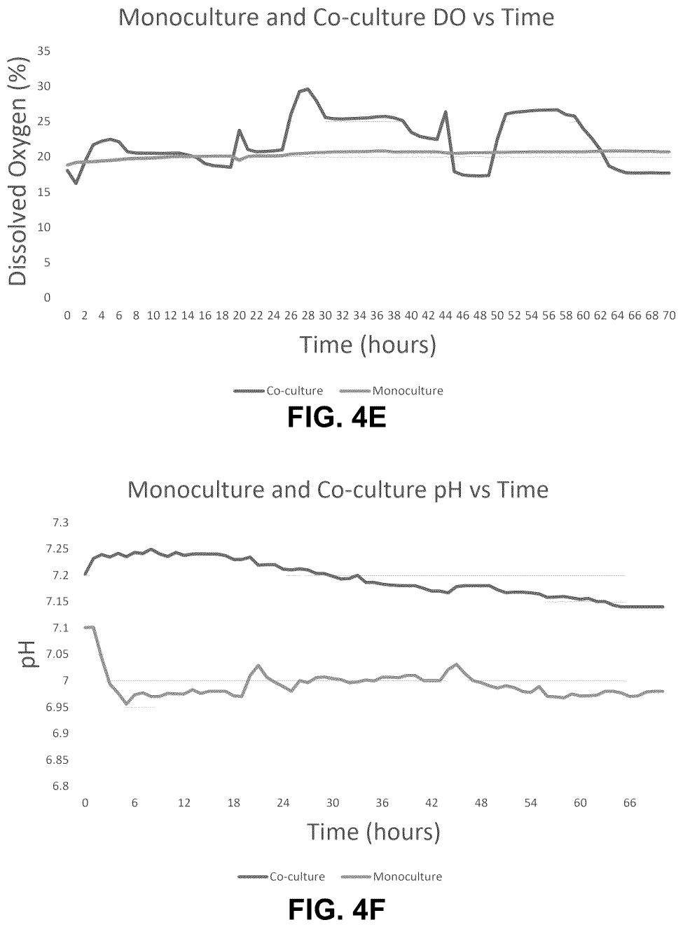

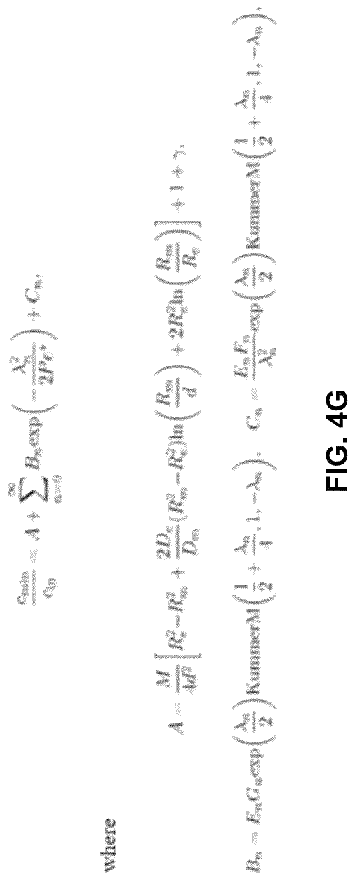

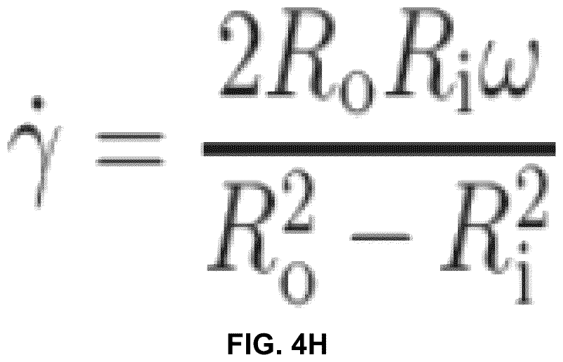

6.1.2.1 Hollow Fiber Reactor: For the hollow fiber bioreactor, the PBSCS will grow on the outside of each fiber. There are 100 fibers in this chamber, with about 80% of the surface area covered with PBSCs. The C. reinhardtii cells (or other cells used) will flow through the bioreactor in the DMEM and will be separated inside the hollow fiber chamber by a semipermeable membrane. This allows the waste metabolites to transfer from the cells while keeping the two cells separate. A depiction of the cells flowing through the lumen is shown in FIG. 4A. FIG. 4A shows a cross-section of the algae cells 135 flowing through a hollow fiber. The process is driven by a pump pushing the DMEM throughout the system. There will also need to be a light source for the algae cells to allow for photosynthesis of the cells to occur. A process flow diagram of the model can be seen in FIG. 4B. FIG. 4B shows a process flow diagram of an overall hollow fiber reactor model. SBI sensors measuring pH and dissolved oxygen (DO) will also be utilized for monitoring the status of the system. One sensor will be placed just before the media enters the hollow fiber chamber and one at the end of the chamber. This can show how each pass through the cartridge changes the DO and if the system can stay at a steady state of oxygen.

6.1.2.2 Hollow Fiber Model: To further model the bioreactor, COMSOL was used to model the diffusion of oxygen inside the lumen. The diffusion rate of oxygen was found to be 2×10−5 cm2/s¹¹⁵. Although this is the value used for oxygen diffusion in water, there was little research into the diffusion rate of oxygen in DMEM or other media, and the group did not have time to test for it. Using this information, the model was created using a boundary value problem of a constant flow rate with no advection or convection in the system. In FIG. 4C is a cross-section of the diffusion of the oxygen in the lumen of a single hollow fiber. Using this model, the group was also able to determine the inlet concentration of oxygen, which is 0.22 mol/m³. FIG. 4C shows a model of oxygen diffusion in the lumen.

6.1.2.3 Governing Equations: To begin with the general calculations of the bioreactor, we first needed to make some assumptions. First. the group assumed the flow rate was constant and there is no advection in the system, as stated above in section 6.1.2.2. The flowing media and algae cells are well mixed for this problem, meaning the algae cells are assumed to not settle at the bottom of the media reservoir. The group also assumed that 80% of the surface area of the fibers was covered in PBSCs, with a 200:1 ratio of algae to PBSCs. We are also currently neglecting the replication rate of both cells. The Navier-Stokes equation, along with the continuity equation, were also good governing equations used for finding flow velocity from density and viscosity. The fibers are each 0.2 meters long and have a diameter of 200 micrometers while the hollow fiber membrane will have an outer diameter of 400 micrometers, also 0.2 meters long, a pore size of 0.1 microns, and be made of polypropylene. Polypropylene is a durable, cost-effective material to use for the semi-permeable membrane and does not contain BPA like some plastics. This means that polypropylene is non-toxic to humans and safe to use in a cell culture system for eventual human consumption.

We calculated the surface area of one fiber to be 252 m², which means 201.6 m² is covered in PBSCs since we assumed a maximum of 80% of the surface to be covered in PBSCs. The area of a bovine cell is about 500 um², which means 403,200 PBSCs are attached to each individual fiber. Since our system uses 100 fibers, this means there are a total of 40,320,000 PBSCs and 8,064,000,000 algae cells within the bioreactor system initially. Using the minimum oxygen concentration in the hollow fiber bioreactor and the concentration of oxygen at the inlet calculated from COMSOL, along with constants in the analytical operating equation, the reduced Peclet number for the fiber was calculated. The Peclet number can then be used to determine the velocity inside the lumen¹¹⁶.

The inside velocity of the lumen was calculated to be about 1.2 centimeters per second. From the velocity, the group calculated the overall flow rate of the system to be 3.77 um²/s. This causes the C. reinhardtii cells to be subjected to a shear force of 1.536 pascals (Example 12). Further research will be conducted on the ability of cells to withstand this shearing force. Further research will also be conducted into the oxygen consumption rate of PBSCs, to determine whether this system will ever need oxygen pumped into the chamber to help make up for the loss of dissolved oxygen as the C. reinhardtii cells die. This likely shouldn't be the case if the media is changed every 4 days, which already doubles the current lifetime of cell culture media, as evidenced from our SBI sensor data.

### 6.2 Evaluation of Design Criteria

Multiple objectives and constraints were determined in Section 3 (above) to guide the design and testing processes. This section will describe how well the final design fulfilled these objectives and constraints.

### 6.2.1 Objectives

6.2.1.1 Scalable: The proposed hollow fiber bioreactor can be scaled up by increasing the number of fibers and the size of the pumps, tubing, and vessels. The benefit of this design is that the hollow fibers provide a large surface area for PBSC growth with a relatively low required volume of media. These bioreactors can take up relatively little space for large-scale production.

6.2.1.2 Proliferation of PBSCs: PBSCs were able to proliferate in co-culture with C. reinhardtii. This was quantitatively shown in FIG. 3G. PBSCS cultured in a 200:1 ratio of C. reinhardtii to PBSCs proliferated more than those grown in a 100:1 ratio and those grown without algae.

6.2.1.3 Harvesting PBSCs: In the bioreactor of the present invention, the PBSCs and C. reinhardtii are separated by a semipermeable membrane. PBSCs can be removed from the scaffold surface to be harvested for differentiation.

6.2.1.4 Reduce Media Cost: The main indicator for replacing cell culture media is a drop in pH. This indicates the level of metabolic activity because higher levels of carbon dioxide and lactic acid will lower the culture pH. As seen in FIG. 3L, after 48 hours of growth (typical time before changing media), the pH of the monoculture was 7.16. The co-culture grown in the same conditions reached a pH of 7.16 after 88.6 hours of growth. Based solely on pH values, the media lifetime was extended by 85%, which means the media can be replaced less frequently. Further testing is necessary to obtain an accurate value of media lifetime, but the results indicate that the co-culture system helps reduce media costs.

6.2.2 Constraints-6.2.2.1 PBSC viability: PBSCs were able to grow in co-culture with C. reinhardtii. As represented in FIG. 3J, the apparent viability is high, with only a few cells stained as dead. Due to the cells detaching, a quantitative comparison of viability was not obtained. However, FIG. 3I shows that there were attached cells in co-culture. Future studies will be done to quantitatively confirm PBSC viability.

6.2.2.2 Maintenance of PBSC Stem-Phenotype: The stem phenotype of the PBSCs was not evaluated due to time constraints during the project. Hoechst staining of co-cultures indicated that some cells had multiple nuclei, which only happens after PBSCs have differentiated. This indicates that not all cells retained the stem phenotype, but more specific testing must be performed to assess the fulfillment of this constraint.

6.2.2.3 Chlamydomonas Viability: C. reinhardtii cultures were successfully cultured and exhibited log-phase growth at 30° C. A C. reinhardtii culture was sustained during a 10-day co-culture with PBSCs, indicating that they can remain viable in co-culture. Viability was not quantified, so future studies should use live/dead staining for C. reinhardtii. For the hollow fiber bioreactor, the effects of shear stress on C. reinhardtii must be explored to confirm that they will remain viable.

6.2.2.4 Chlamydomonas Functionality: The literature suggests that phenol red may inhibit the O₂ production of C. reinhardtii, so DMEM without phenol red was used for the co-culture system. In co-culture, C. reinhardtii increased the dissolved oxygen concentration during the 12 hours when lights were on. This indicates that C. reinhardtii retained its metabolic activity while in co-culture grown in DMEM.

6.2.3 Evaluation of Standards: The goal of this bioreactor system is to be able to produce large quantities of cultured meat, so it is important to look at the design and manufacturing standards laid out earlier in the paper. Cell cultured meat is a generally new field and not all regulations are in place, but it is important to discuss the bioreactor standards as well as the meat product standards.

6.2.3.1 Bioreactor Standards: The hollow fiber bioreactor design meets ISO 11737-2 because this is a closed system that allows for little contamination risks. The main risk of breaking this regulation comes when changing the media or harvesting the PBSCs, which is easily mitigated with safe and sterile lab practices. The equipment can also be emptied and sterilized in an autoclave. The other design regulations can be followed with property industry safety and handling standards.

6.2.3.2 Meat Production Standards: One important USDA standard for meat production shown earlier is pathogen intervention. Section 315 states there must be at least two pathogen intervention steps. The current design of the hollow fiber bioreactor is mostly self-sufficient and has little human interaction which decreases contamination risks. There could also be a sanitization of the equipment after each use to help mitigate the risk. Gloves and other necessary equipment will also be used in a sterile environment around the apparatus to help with this too.

Another important standard is discussed in section 318.7, which is microbial testing. This mostly pertains to the meat produced by the bioreactor, and there are several ways to ensure no microbes are in the meat product. Microbial contamination changes the color of the meat, so this is one obvious way¹¹⁷. Another idea is the use of a pH sensor in the media, which can show if the media is contaminated as well. The FDA does not regulate the beef industry but does have some regulations, such as having a hazard analysis and risk preventative control guide. This is for the meat production factory owner, and not for the small-scale version our group has made. This would, however, be followed in a scaled-up factory version with a plan to mitigate risks and hazards when working with the machinery and the dangers to the public that may come from mismanaging the meat.

### 6.3 Broader Considerations

6.3.1 Economics: Cellular agriculture must be able to compete with traditional meat production on a large scale. The cost of cell culture media in large-scale production is prohibitive, but this co-culture design can alleviate some economic hurdles. As this system is refined and combined with other cost-cutting measures, cellular agriculture can become economically feasible to meet the rising global demand for animal products.

6.3.2 Environmental Impacts: The environmental impact of meat production is evident and explained further in Section 2 above. Cellular agriculture offers a potentially sustainable alternative, but advancements in technology are necessary to make it truly sustainable. Life cycle analyses are required to ensure that all parts of the process are accounted for. The co-culture system offers a way to reduce water and energy usage by reducing the need to produce, transport, and replace cell culture media.

6.3.3 Societal Influences: While the demand for meat is expected to double in the next half-decade, there is not enough land to meet the necessary supply. Animal agriculture requires wide plots of land, especially with a societal rise in concern for animal wellbeing. While factory farms aim to reduce the need for land, there are numerous ethical and safety concerns that are discussed in Section 2 above. The benefit of cellular agriculture is that meat can be cultivated indoors with minimal environmental requirements. Meat could potentially be produced in ‘vertical farms’, which use skyscrapers to increase the amount of meat produced per area of land. Specific climate and vegetation are not required, so the locations that meat can be grown will be expanded. This will increase access to calorie-dense food throughout the world while decreasing land usage and transportation costs.

6.3.4 Political Ramifications: There is already pushback from large meat producers against the integration of cultured meat products into the market. Lobbyists will continue to push legislators to outlaw the term ‘meat’ when describing cellular agriculture products. This will be a difficult hurdle to surmount, so it is necessary to start educating lawmakers and their constituents on the reality of cellular agriculture. The cells used for cultured meat products are genetically identical to the cells that make up traditional meat; they just do not require the death of an animal to produce.

6.3.5 Ethical Concerns: Cellular agriculture has the potential to obsolete many careers in the meat production industry. These ramifications cannot be ignored, especially since a goal of cellular agriculture is to better the conditions of humankind. Cellular agriculture researchers must engage in conversations with farmers, meat producers and distributors, and other workers in the field. It will be important to find ways to integrate cellular agriculture with some current practices to prevent the destruction of livelihoods. When this is not possible, there must be free access to education and retraining so those affected can continue to contribute to society and support themselves financially.

6.3.6 Health and Safety Concerns: Animal-borne illness is a major concern in modern society. Novel diseases like COVID19 and the swine and avian influenzas are becoming more common due to our animal agriculture practices. The widespread use of antibiotics in factory farms increases the chance of these novel pathogens being drug-resistant. The safety of our society can be increased by shifting away from our reliance on factory farming. Cellular agriculture offers an alternative that would reduce the chance of spreading novel pathogens to the human population.

6.3.7 Manufacturability: The success of this design is only possible if it can be scaled for the mass production of cultured meat. The hollow-fiber bioreactor design can be scaled up by increasing the number of hollow fibers and the size of pumps and vessels. As the large-scale manufacturability of cellular agriculture products increases, cultured meat will become a more competitive option when compared to traditional meat. Due to the potential for reduced land, water, and energy usage, large-scale cellular agriculture can surpass the meat-producing capabilities of traditional animal agriculture.

### 7. Discussion

7.1 Ideal Co-culture Conditions: Various co-culture conditions were investigated throughout the experimental process to determine the most ideal growth conditions for co-culturing PBSCs with C. reinhardtii. Through the different experiments, the established conditions for ideal growth of both cell types were to use DMEM (without phenol red), at 30° C., with a 12-hour on/12-hour off light cycle, with no agitation. Although the current co-culture condition allows for PBSC proliferation with minimal cell death while extending the media lifetime by 85%, further experimentation is needed to fully investigate each parameter.

7.1.1 PBSC and C. reinhardtii viability in co-culture media: The results of the experiments shown in Section 5.2 suggest that the ideal conditions established for the co-culture are not the most ideal for C. reinhardtii. The components present in DMEM do not pose any toxicity to C. reinhardtii, but DMEM does not provide any nutrients the algae need to proliferate. The inability to proliferate in DMEM is an issue that could be potentially solved by adding certain salts and heavy metals that are present in TAP media. However, the addition of these salts could be detrimental for the PBSCs; therefore, further experimentation will be needed.

The option of using different culture media should also be considered. Our team has initially tried to investigate whether there is an optimal DMEM: TAP ratio that is conducive for both PBSC and C. reinhardtii growth. Due to serum contamination and the inherent large amount of variability, the experiment was put on hold. Nevertheless, it should be recognized that although the experiments did not yield any conclusive results, the data did suggest that high ratios of TAP are not ideal for PBSC viability and proliferation. There are varieties of other media that could be used for algae growth, such as plant food, F/2 media, and BG-1; therefore, other algal media should also be explored in the future to properly assess whether a mix of DMEM and algal media could be used to achieve proliferation of both cell types.

An in-depth analysis of the media composition is another avenue of research that should be explored in the future to determine the ideal co-culture media composition. In this experiment, our group's focus was on the carbon and nitrogen cycle. However, there are other media components that need to be considered when trying to extend media lifetime, such as the production of lactic acid, the depletion of growth factors, etc. Our team hopes that an investigation on the composition between the new media, spent media and the media generated from the co-culture system will reveal other critical waste by-products that need to be recycled.

7.1.2 C. reinhardtii agitation: The usage of stir bars for the agitation of algae has also been proven to be unnecessary. Data have indicated that the usage of a stir bar has resulted in higher clumping as well as lower cell proliferation compared with no stir bar. The phenomenon observed in the stir bar condition could be potentially explained by the blunt trauma or force experienced by C. reinhardtii. The purpose of shaking the algae culture is to allow for better aeration and allow the cells to receive proper lighting by de-clumping the algae culture. However, the introduction of the stir bar did not achieve the desired results and caused more clumping. The standard procedure for algae agitation is usually achieved through a shaker. The usage of a shaker would negate the possibility of blunt trauma/force affecting the viability and proliferation of the algal cells. This experimental procedure was not explored during the experimental process due to technical constraints of the oxygen and pH readers, but it remains a possible option to further improve the co-culture conditions.

7.1.3 Possible improvements: It should be noted that there are still many different variables that could be explored to improve the co-culture conditions, such as the lighting conditions. The 12-hour on-off light cycle was a decision made after literary research, but no experiments were conducted to verify whether this was the most optimal condition. The establishment of the 12-hour light cycle was to try and maximize the photosynthetic capability of C. reinhardtii without bleaching them with too much light. Further literary research has suggested the usage of strobe LED lights could potentially increase the photosynthetic ability of the algal culture as well as mitigating light attenuation¹¹⁸. The experimentation with different lighting conditions is a variable that should be examined to improve the current co-culture specifications.

As aforementioned, the usage of a media analysis could also potentially suggest ways to improve the culture media. By assessing what components are vital or detrimental for the PBSCs and C. reinhardtii, a creation of a new media would be theoretically possible. The current system allows for PBSC proliferation with high viability; however, this comes at the cost of reducing C. reinhardtii growth. Therefore, the creation of a media that allows for growth of both cell types could potentially reduce the problems of algae maintenance in a large-scale setup.

### 7.2 Pbsc—C. reinhardtii Culture in Small Scale

Throughout the design process, the establishment of an ideal ratio is a vital process that will allow the team to determine the best way to scale-up the bioreactors. From the results, we suggest that a 1:200 ratio of PBSCs to C. reinhardtii allows for the highest rate of proliferation for the PBSCs. The Hoechst staining of the PBSCs in different algae ratios revealed that the algae, in the 1:200 ratio, was able to produce enough oxygen in the system to allow for PBSC proliferation. After three days, the control only had half of the initial 50,000 cells plated per well. The 1:100 ratio exhibited no doubling but no decrease, while the 1:200 ratio exhibited a single doubling in the three-day culture period. In addition to PBSC proliferation, both the PBSCs and C. reinhardtii have appeared to survive well in the DMEM conditions with light present with a 12 hour on/off light cycle.

7.2.1 SBI DO and pH Sensors: Our results from Section 5.3 suggested that PBSCs were viable, and algae provided enough oxygen to support the growth requirements of PBSCs. The pH was buffered more compared to the control culture, and the dissolved oxygen was higher than the control. The successful increase in viability of PBSCs as the ratio of algae to PBSCs increased suggested that there could be higher ratios explored to determine at which point PBSCs experience toxic effects. Media analysis would be needed to quantify the impact of the algae directly on the molecular components of DMEM.

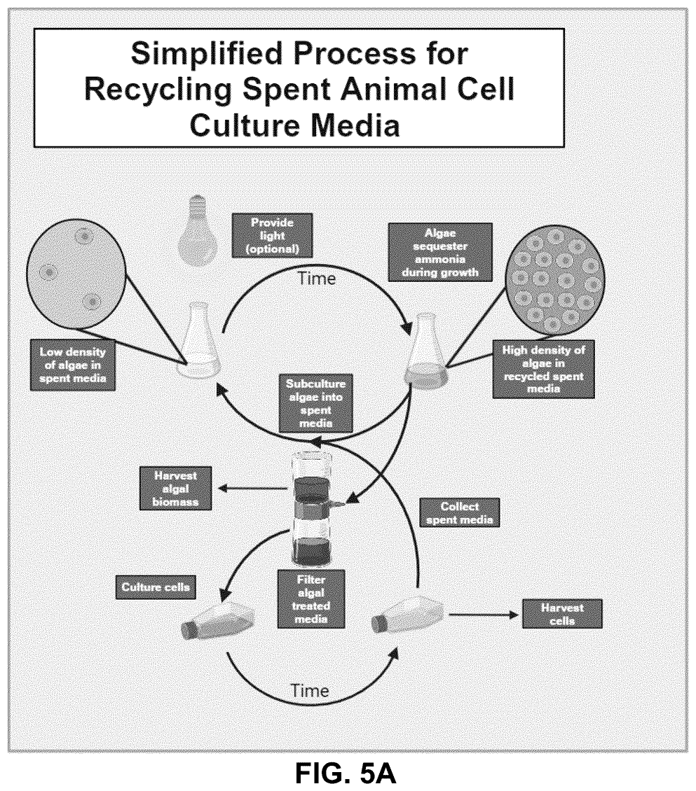

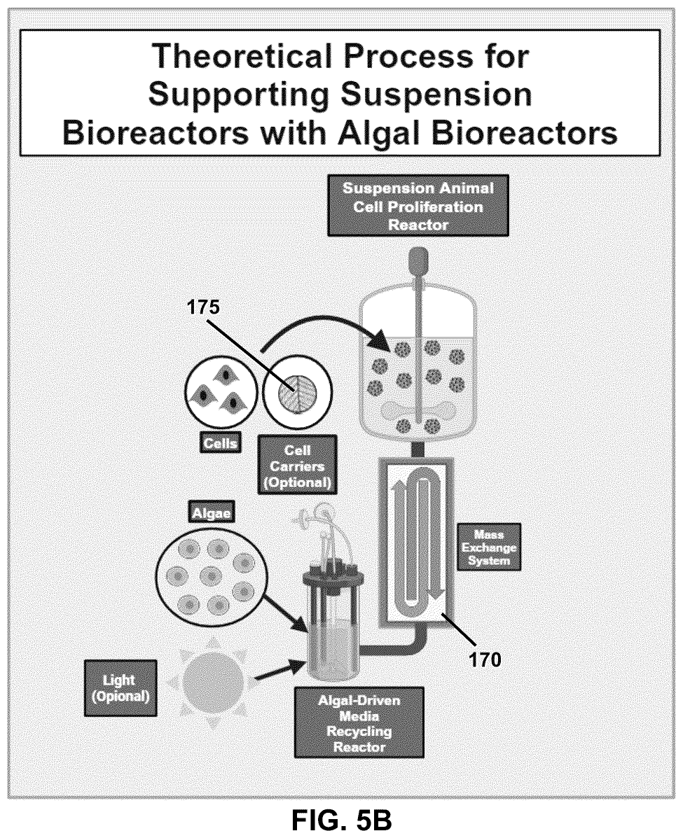

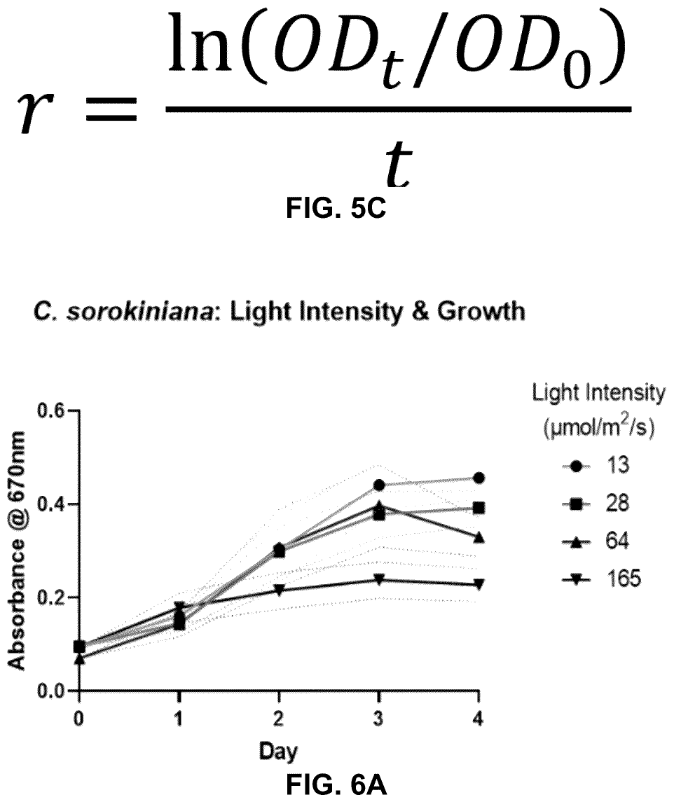

The DO increase with algae presence suggested that the algae created oxygen and thus more suitable environments for PBSC growth. The initial increase in the DO of the first 3 days of the 10-day period in the figure describing the pH (FIG. 5.12) can likely be explained by the high productivity rate of algae in the first few days of co-culture. This could perhaps be because the PBSCs also became less metabolically active to produce carbon dioxide as their media became depleted of resources. The DO values also fluctuated significantly between 12-hour light cycles, which suggest that algae have very different oxygen production when they are not under lights. In a larger scale system, the algae productivity as it flows through the hollow fibers of the reactor will have to be considered, since the algae will be moving away from the light source. It should be evaluated in future experiments whether the DO or the pH contributes more to the survival of PBSCs. It was also not determined whether these results were statistically significant compared to the monoculture, however results were consistent among many trials although the trials could not be directly compared.

Additionally, the buffer in pH could be attributed to many different factors. The pH may have decreased due to limited availability of oxygen after algae became less metabolically active after three days. The lack of oxygen would contribute to a decrease in cellular respiration and an increase in fermentation, which would contribute to the production of lactic acid. Certainly, the conditions were not hypoxic, so it could not be confirmed that fermentation was occurring, and lactic acid was present. It is likely that overtime, the algae could not metabolically compensate for all the carbon dioxide that the PBSCs were producing. Overall, there were limitations with the experimental setup of the sensors and readers which will be discussed more in the next section.

Other questions that remain are whether the algae would be capable of recycling nitrogenous wastes of PBSCs such as ammonium. The team was unable to directly test the presence of ammonium and nitrogenous products over the course of the experiments which evaluated DO and pH. These data would help suggest whether algae could compensate for significant waste production of PBSCs and replenish media.

An additional question is whether growing the algae directly in place with the PBSCs could have contributed to growth inhibition of the PBSCs since algae were taking the physical space of the PBSCs. Alternatively, the algae in direct contact with the PBSCs may have provided for very proximal metabolic conversions of wastes to sources for PBSCs, specifically carbon dioxide to O₂. The potential inhibition should be accounted for in the hollow fiber model, and the distance from the algae cells will still be very proximal to PBSCs in the model.

7.2.2 Transwell: In the Transwell experiment, the staining protocol did not provide the expected results. The expected viability of 90% may be slightly lower than the actual value. The PBSCs detached from the culture flask in which they were being stained. Perhaps the cells reacted poorly to the live/dead stains, since in previous experiments the cells had not been detached when Hoechst stains were conducted in the same culture conditions. The DMSO in the Calcein AM and Etd-1 stains were at such low concentrations once the stocks were diluted in PBS that it would not have been probable for DMSO causing cell detachment and thus cell death. The cells were not dead however and could have simply detached since they were at room temperature for 30 minutes without any suitable culture medium.

Staining the PBSCs with Ki67 for proliferation was another goal of the project that could not be conducted due to time and COVID-19 related restrictions. Ki67 stains would have informed with precision whether PBSCs were proliferating or differentiating and would have quantitatively proved that we met our original objective of ensuring that PBSCs were proliferating and not differentiating into other bovine muscle cell types. Although these stains were not conducted, qualitative analysis of the cell morphology informed the team that there was minimal differentiation and significant proliferation of the cells as PBSCs.

7.3 Current Limitations of the Experiments: The ID reader software and hardware that the team used to collect DO and pH was new to our team and our advisors. We worked with the manufacturer to improve our knowledge of the sensors, software, and best practices. Initially, the team struggled with accurately placing the small sensors due to issues with the adhesive adhering to the original packaging and not removing properly. This impacted the integrity of the sensor and could have contributed to the accuracy of the sensors to give a proper reading throughout experiments. In the last few experiments, the sensors with technical adhesive issues were replaced with sensors that could be removed from packing more easily. This proved to remove noise from the graphs and provided more consistent results.

The readers had other technical issues due to interference of the LED lights with the reader's mechanism of collecting data. The reader utilizes small LEDs to emit light which interact with the sensors of the culture flask of interest and determine the DO or pH of the culture. The lights interfered with the ability of the LEDs in the reader to accurately acquire proper data points, so data collected during the 12 hour “on” light cycle was slightly skewed. The data could be compared between 12 hour “off” light cycles, and separately compared between 12 hour “off” light cycles.

If the experiments required taking photos of the culture flasks, there was also some uncertainty that the sensors would be misaligned upon being replaced on the readers in the incubator. The team adapted to this uncertainty for the data shared in this report by using separate culture flasks which were held at the same conditions as the flasks on the readers.

Beyond the technical limitations of some of our experimental set-ups, the gas exchange could have not been entirely blocked for the Transwell and ID reader experiments. Parafilm does allow for some gas exchange which over short intervals of time may not have significant impacts however our experiments over 3 to 10 days may have been impacted¹¹⁹.

### 8. Conclusion

This project culminated in the creation of a proof-of-concept for an algal co-culture system to decrease the cost and environmental impact of large-scale cellular agriculture. The results support the hypothesis that C. reinhardtii co-cultures can increase PBSC proliferation in hypoxic conditions. This was assessed with Hoechst staining of co-cultures separated by Transwell inserts. The two species were also co-cultured without separation, and measurements of dissolved oxygen (DO) show that C. reinhardtii increased the DO during light cycles. This result indicates that the microalga can remain metabolically functional in co-culture with PBSCs grown in DMEM. The pH readings show that C. reinhardtii slowed the pH drop of the coculture, which supports the hypothesis that the algal co-culture can extend the lifetime of cell culture media. Live/dead staining was used to assess the viability of PBSCs in co-culture, and the results indicate that PBSCS can retain high viability after three days of culture with C. reinhardtii. These data indicate that C. reinhardtii co-cultures should continue to be explored as an avenue for media recycling in large-scale cellular agriculture. A conceptual design of a hollow-fiber bioreactor for scale-up of the co-culture was modeled, although further work is necessary to prototype and validate the design.

Future studies should aim to confirm if the PBSCs retain their stem-phenotype in coculture since this is necessary to maximize the proliferative potential of the cells. The addition of growth factors from a sustainable and low-cost source is a technological advancement that can help achieve this cell culture constraint. This project only studied co-cultures with a 12-hour light cycle, so the exploration of additional light conditions would generate valuable data for optimizing the co-culture system. The literature suggests that C. reinhardtii can uptake ammonium from the media, but time and monetary constraints prevented the team from confirming this experimentally. Measuring ammonium concentrations will be necessary for future studies, as well as characterization of other media components. A complete spent media analysis of various salts, amino acids, small molecules, and proteins will help characterize the full effects of the co-culture system. Lactic acid is a major waste metabolite that is not addressed with C. reinhardtii co-cultures. Therefore, the next step in developing a media recycling coculture system is identifying and testing an additional cell species that can metabolize lactic acid.

Ecosystems for Bioprocessing: Recycling Spent Animal Cell Culture Media Using the Thermally Resistant Microalga Chlorella sorokiniana.

Cell culture media is a significant contributor to the high cost of bioprocesses for stem cell therapies, or for cellular-agriculture. These additional studies explore the potential of algae to re-condition spent cell culture medium. We investigated the growth and ammonia consumption by Chlorella sorokiniana, a thermally resistant microalgal species in spent QM7 cell growth media at 37° C. Algae was grown under variable light intensities with the greatest difference in growth occurring between 13 and 165 μmol/m²/s (Optical Density (OD) measured at 670 nm: 0.23±0.08 and 0.46±0.10, respectively). Improved growth was observed when C. sorokiniana was grown heterotrophically in the dark, vs. mixotrophically in the light (OD 0.92±0.37 and 0.40±0.20, respectively). More rapid algal growth was observed in four days spent QM7 cell media, when compared to fresh media (OD of 1.39±0.22, 0.47±0.17). Within 72 hours, nearly all ammonia and glucose were eliminated from both high- and low-density algae inoculations (100 and 98% glucose, respectively; and 98 and 99% ammonia, respectively). No cytotoxic effects were observed on QM7 cells grown in algal-treated growth media. QM7 cells exhibited better metabolic activity in algal-treated spent medium than in untreated spent medium (44.57±10.82 and 80.85±12.02 percent activity of fresh media group, respectively). These results suggest that C. sorokiniana can be grown in spent cell culture media, at 37° C., and potentially extend the lifespan of media thereby enabling more affordable bioprocesses.

### INTRODUCTION

The products of modern animal cell bioprocesses, such as monoclonal antibodies, therapeutic proteins, cell therapies, tissue engineered grafts, and cultured meat have the potential to radically contribute to satisfying global pharmaceutical and food challenges¹²⁰⁻¹²³. The realization of cultured meat systems alone may contribute to meeting all 17 of the Sustainable Development Goals¹²⁴. While some of these technologies are currently regulated, marketed, and employed, their costs and availability limit their access for large populations. For example, as of 2018, the upfront drug cost of chimeric antigen receptor T-cell (CAR-T) therapy, a next generation cancer treatment, excluding costs associated with clinical support, was estimated at $373,000¹²⁵. Similarly, cellular agriculture, or the in vitro manufacturing of agricultural products, primarily meat, may aid in reducing the environmental strain of traditional livestock systems while providing a reliable source of affordable animal protein to large populations. As of 2023, some cultured meat manufacturing processes have been proposed and received regulatory approval, yet there currently exists no widely available cultured meat product marketed in the US that is economically competitive with traditional meat^{126,127}. The development of innovative, cost-effective strategies for scaled animal cell bioproduction is necessary to minimize the costs of current and future therapies, and to realize the potential for cellular agriculture to diminish the environmental risk of our food production systems.

Cell culture media is vital for supporting animal cell growth and metabolism in such bioprocesses¹²⁸. Medium typically contains sugars, vitamins, minerals, hormones, amino acids, proteins, and growth factors, among many other potentially vital components that are unique to meeting the demands of a particular cell type. As animal cells divide and metabolize in large scale expansion bioprocesses, low concentrations of cellular waste, such as ammonia, accumulate in the cell culture media¹²⁹. Eventually, a concentration threshold is reached, at which point cells exhibit characteristics that are detrimental to further cell growth. During controlled manufacturing processes, cultures are commonly maintained under this concentration threshold by continually adding fresh media to the culture, thus increasing the working reactor volume in fed-batch processes¹³⁰. While modern approaches to on-line analysis of nutrients in media, such as Raman spectroscopy, allow for design of more efficient and productive feeding strategies, upon reaching the concentration threshold at maximum volume capacity, the costly media is disposed of as waste¹³¹.

Current analyses conclude that cell culture media is the most expensive input in a cultured meat production process. One such analysis suggested that, at current commercial prices, the serum free media formulation, Essential-8, would cost about $400 per liter, and about $7,535,958 per 20,000 liters¹³². At such costs, a batch production strategy employing a 20,000 L tank would yield meat at a cost of >$8,600/kg. Additional scenarios are reasonably proposed, which consider theoretical implementation of strategies for decreasing costs, such as manufacturing growth factors at scale and preconditioning of cells. Advanced scenarios suggest, even with high media use, the medium raw material cost contribution of meat could range from $9.60-$34.00/kg. While recent developments in media technology, such as demonstrated use of recombinant albumin, continue to reduce the projected costs of bulk media for cellular agriculture processes, the development of new strategies for extending the lifespan of cell culture media may also further decrease production costs.¹³³

FIG. 5A shows a simplified process for recycling spent animal cell culture media. FIG. 5B shows a theoretical process for supporting suspension bioreactors with algal bioreactors. As the media is metabolized by the animal cells, it would be continuously pumped through a mass exchange system 170 designed to enable media to enter a secondary microalgal reactor without any cross transfer of animal or algal cells. A mammalian cell carrier 175 can be used to culture mammalian cell upon. In some aspects of the invention, the mammalian cell carrier 175 can be a fixed mammalian cell substrate. The metabolic plasticity of microalgae is well documented by extensive study of their potential applications in biofuel production and wastewater treatment^{134,135}. Their capacity for biomass growth is conserved when subjected to a wide range of both media constituents and general culture environments. Experiments confirm their ability to sequester nitrogen originating from ammonia, suggesting their potential capacity to eliminate it from animal cell culture media¹³⁶. Haraguchi et al.¹³⁷ hypothesized that algae can eliminate ammonia that has accumulated in cell culture media after murine C2C12 myoblast cell expansion. Within their proposed process Chlorococcum littorale, a marine unicellular green alga, sequestered nearly all ammonia from the spent animal cell culture media. Within this bioprocess, the spent cell culture media was treated by C. littorale at room temperature, within a light controlled, room-temperature environment, as constrained by the environmental requirements of the alga. It is important to note that satellite cells, a commonly proposed cell type for cultured meat products, are traditionally cultured at 37° C. in DMEM containing 10% FBS and buffered at a pH of 7.4. Thus, it would be advantageous to identify a species of algae better suitable for growth in the cell culture environment¹³⁸. The identification of a species capable of growth and ammonia removal in this cell culture environment may enable transition from batch treatment of spent media to a continuous approach. Within an integrated bioreactor supporting algae growth and mammalian cell growth kinetics can be modeled and monitored in concert.

While most photosynthetic algae, such as C. littorale, are typically cultured at much lower temperature and in more alkaline conditions than animal cell cultures, other algae species have adapted to more extreme environments¹³⁹. The freshwater alga, Chlorella sorokiniana has been observed growing in a wide variety of, sometimes extreme, environmental conditions in nature, suggesting that it may maintain the ability to be conditioned to grow in more saline and complex solutions such as DMEM at 37° C.¹⁴⁰⁻¹⁴². In laboratory environments, this strain has been studied for its potential in wastewater treatment, fermentation, and biofuel production applications. Previous literature demonstrates its growth at temperatures up to 40° C., at pH ranges between 5.8 and 9, suggesting a unique opportunity to grow C. sorokiniana within the same environmental conditions as animal cells¹⁴³. Additionally, C. sorokiniana has metabolic plasticity to grow both photoautotrophically, in the presence of light, and heterotrophically, in the absence of light when carbon sources, such as glucose or acetate, are locally available in the growth medium¹⁴⁴. If the provision of light can be eliminated within a theoretical waste-media management system, this evolutionary advantage may enable light independent culture media systems driven by continuously supplemented glucose.

Previously, we sought to improve the affordability of tissue engineering and bioprocessing for cellular agriculture by incorporating materials inspired by nature, crossing-kingdoms between plant tissues and animal tissues; notably, via use of decellularized plant tissue such as spinach to support of human pluripotent stem cell-derived cardiomyocytes, and broccoli and agricultural waste such as corn husk and jackfruit rind as cell carriers in suspension cultures¹⁴⁵⁻¹⁴⁷. Continuing this theme, we propose the incorporation of C. sorokiniana, as an organism in parallel culture with animal cells, into mass-bioproduction systems to enhance production capacity aimed at reducing the cost of the most expensive input, cell culture media.

In this study, we hypothesized that C. sorokiniana would grow in media designed to support animal cells at 37° C. and could eliminate ammonia from spent cell culture media to enable retention of animal cell metabolic activity. Growth rates of algal cultures in algal media and animal cell growth media were compared under variable light conditions. The cytotoxicity of algal-treated animal cell culture media was measured. The rate at which ammonia and glucose were removed from the media was observed and the metabolic activity of animal cells grown in fresh, spent, and algal treated spent animal cell growth media was compared.

### 2. Materials and Methods

2.1 Algal Cultivation: Chlorella sorokiniana UTEX1230 was acquired from the Culture Collection of Algae at The University of Texas at Austin. The culture was subcultured and maintained in Tris-Acetate-Phosphate medium (TAP) (PhytoTech Labs) at pH 7.4, in 125 mL Erlenmeyer flasks, on an orbital shaker at 100 RPM, at ˜ 60+/−5 μmol/m⁻²/s on a 12-hour day/night cycle using an USHIO-UFL, F32 T8/850, CRI85 linear fluorescent light bulbs, for a minimum of 5 days at 37° C. before inoculating into fresh TAP media at a density of 500,000 cells/mL. Microalgae were subcultured into fresh TAP media approximately every 7 days to maintain actively growing cultures. Algae were grown for at least 5 days prior to initiation of each experiment.

2.2 Cell Culture: QM7 Cells (CRL-1962 ATCC), transformed quail myoblasts, were acquired and cultured at 37° C. and 5% carbon dioxide in growth media (GM) (DMEM/F12 (Thermo Fisher Scientific), 10% heat-inactivated fetal bovine serum-FBS (Thermo Fisher Scientific), and 1% Penicillin and Streptomycin (Thermo Fisher Scientific)). Cell culture medium was replaced every 48-72 h during maintenance culture and cells were subcultured upon reaching approximately 80% confluence.

2.3 Imaging Microalgae: Microscopic images of C. sorokiniana were taken after 7 days of growth in GM using an Axioimager Z2 microscope (Zeiss, Oberkochen, Germany). Brightfield images were taken and overlaid with fluorescent images taken at 649 nm/667 nm excitation/emission to observe the conservation of chlorophyll within the chloroplasts after extended culture in GM. ZEN 3.4 Blue Edition© imaging software (Carl Zeiss Microscopy) was used for image processing.

2.4 Spent Animal Cell Culture Media Preparation: To prepare stock quantities of spent animal cell culture media, 500,000 QM7 cells were inoculated into T-75 tissue culture flasks containing 10 mL of GM. Cultures were maintained without media change or subculturing for 2, 4, 6 or 8 days. Following 4 and 8 days of culture, the media was collected and frozen at −80° C. and considered spent growth media (4D-SGM and 8D-SGM, respectively).

2.5 Algal Growth Under Variable Light Intensities: Algae were inoculated at 500,000 cells/mL into 125 mL Erlenmeyer flasks containing 10 mL of fresh GM. Flasks were placed in the incubator at 37° C. under light intensities of approximately 13, 28, 64, and 165 μmol/m2/s, measured by a LI-COR light meter (LI 250A),) equipped with a quantum sensor. Algal growth was monitored daily for 4 days using 3 averaged absorbance readings at 670 nm using a Victor Nivo Multimode Plate Reader (Revity). Flasks were wrapped in aluminum foil for measurement of heterotrophic dark growth.

2.6 Cytotoxicity of Algal-treated Media: Algae in stationary phase, 5-7 days post subculture, were inoculated into 10 mL of fresh GM to establish an initial density of 500,000 cells/mL for 4 days before being removed from the media via vacuum filtration. Approximately 100,000 QM7 cells were inoculated into each well of 6 well tissue culture treated plastic (Corning, ref. 353046) plates in fresh growth media. After 24 h the GM was gently aspirated out of each well and replaced with the algal-treated media. Control groups were given fresh growth media. Cells were incubated at 37° C. and 5% carbon dioxide for 72 h. Experimental cells were stained with Hoechst, Calcein-AM and Propidium Iodide (Thermo Fisher Scientific) before being fixed in 4% paraformaldehyde. A positive control for dead cells was prepared by exposing additional groups to 70% ethanol for 20 minutes prior to staining. A minimum of three images were taken per group using a Cytation-1 microscope (Agilent). The percentage of Hoechst-stained cell nuclei expressing positive signals for propidium iodide was calculated using ImageJ.

2.7 Algal Growth in Spent Animal Cell Culture Media: To identify if C. sorokiniana could grow in spent animal cell growth media, algal cells were inoculated and growth quantified daily for 7 days in 125 mL Erlenmeyer flasks containing GM, 4D-SGM, 8D-SGM, and TAP media in the environmental conditions previously described. FIG. 5C shows equation 1, which was utilized to calculate growth rates. Growth rates were calculated with equation 1 as follows; where r is the growth rate (1/day), ODₜ refers to the optical density at a given timepoint post inoculation, OD₀ refers to the optical density immediately following initial inoculation, and t is equal to the elapsed time since initial inoculation.

2.8 Quantification of Ammonia, Glucose, and Glutamine in Algal-treated Media: Algae were inoculated into 4D-SGM at densities of 10⁷ and 10⁸ algal cells/mL, after counting with a hemocytometer. One mL media samples were taken daily from each group and filtered to remove cells using a 0.22 μm pore sized polyvinylidene syringe filter (Merck). Additional ammonia and glucose measurements were taken at 3 and 7 hours post inoculation for the 10⁸ group. Total ammonia and glucose were measured using a CEDEX Bioanalyzer (Roche) using an automated enzymatic photometric assay using glutamate dehydrogenase (Roche) and an automated enzymatic photometric assay using hexokinase (Roche).

2.9 Metabolic Analysis of Animal Cells Cultured in Algal-treated Media: A 10⁸ aliquot of algal cells/mL was inoculated into 8 to 10 mL of 8D-SGM and maintained at 37° C., at 60±5 μM/m²/s light with a 12 h photoperiod for 24 h to generate algal-treated 8 day spent media. 10,000 QM7 cells were inoculated into 96 well plates in 100 μl of fresh GM. After 24 h, the GM was replaced with 100 μl of fresh GM, 8D-SGM, or algal-treated 8D-SGM. Cultures were maintained for 24 h before analysis via MTT assay (Thermo Fisher Scientific). Briefly, 10 μL of 12 mM solution of 3-(4,5-dimethylthiazol-2-yl)-2,5-diphenyltetrazolium bromide was added to each well and maintained for 4 hours at 37° C. From each well, all but 25 L of media were removed and mixed with 50 μL of DMSO. After 10 min incubation at 37° C., wells were mixed again, and absorbance was read at 540 nm using a PerkinElmer Victor3 spectrophotometer (PerkinElmer). The absorbance of three wells were averaged per group to yield a data point.

2.10 Statistical analysis: GraphPad Prism (Version 9.3.1) was used for all statistical analyses. Mixed effects analysis with post hoc Tukey's multiple comparisons test was used for analysis of C. sorokiniana growth between variable light intensities, between light and dark conditions, and for growth in spent animal media. A Brown-Forsythe and Welch ANOVA with post-hoc unpaired t-tests with Welch's correction was used for analysis of glucose and ammonia consumption by QM7 cells and analysis of MTT assays. Unpaired two-tailed t-tests were used to compare cell viability and percent ammonia and glucose consumption between algal inoculation densities at each day. One, two, three, and four asterisks denote a p≤0.05, 0.01, 0.001 and 0.0001, respectively. The term, ns, indicates no significance. Absorbance values were recorded three times per algal culture at each timepoint to represent the growth of that culture. All algal growth experiments were twice repeated with 2-3 flasks per experiment for a minimum of 5 total flasks observed per condition. Absorbance readings were measured three times per flask at each time point and averaged together to yield a single data point. Each live/dead and metabolic assay was performed on three separate occasions. On growth curves, standard error (SE) is represented by dotted lines colored to match its corresponding mean growth curve, represented by solid lines.

### 3. Results

3.1 Light Intensity and C. sorokiniana Growth: Prior to determining the effect of algal metabolism on the growth of QM7 cells, baseline growth kinetics of C. sorokiniana were identified. Algae inoculated into fresh GM were submitted to different light intensities and growth was measured over four days (FIG. 1a). By day four, growth slowed or ceased. Significant differences in final algal density were observed between 165 and 13 μmol/m²/s (0.23±0.08 and 0.46±0.10, respectively), and 28 and 13 μmol/m²/s (0.41±0.10 and 0.46±0.10, respectively) (FIG. 1b).

Because many algae can grow heterotrophically, algae were inoculated into GM and maintained in either the light (60±5 μmol/m²/s) or the dark and grown for 7 days. After 3 days, the average culture density reached the stationary phase for both conditions. Dark-grown culture cell density was more than double that of the light-grown cultures (FIG. 1c-d). The growth rate of the dark-grown cultures was about twice that of the light-grown cultures (FIG. 1c). After 7 days, the average culture densities did not change. Statistically significant differences in culture density were observed between groups at days 3 and 7.

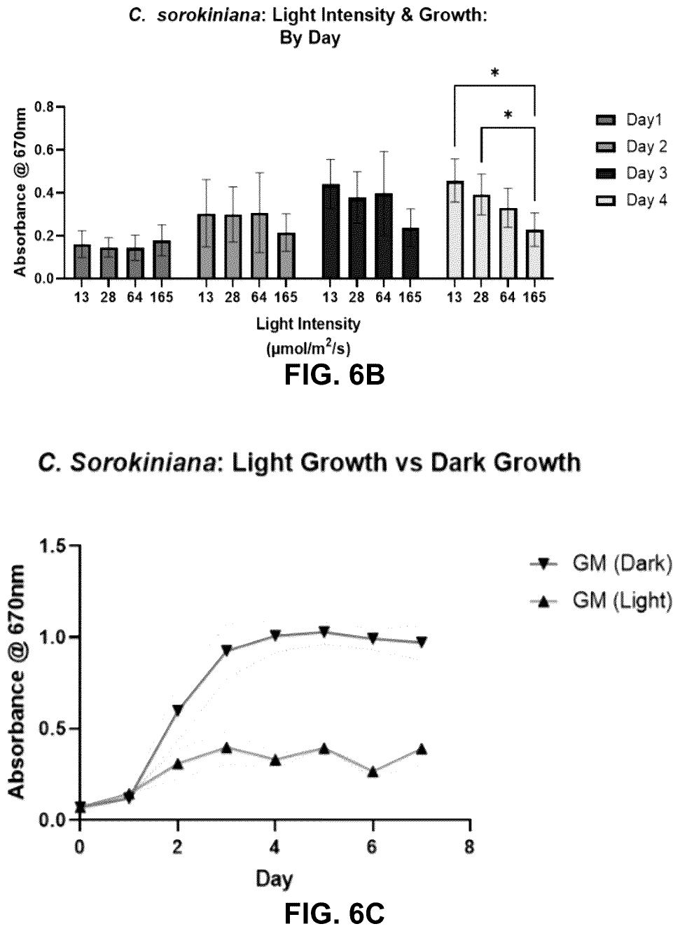

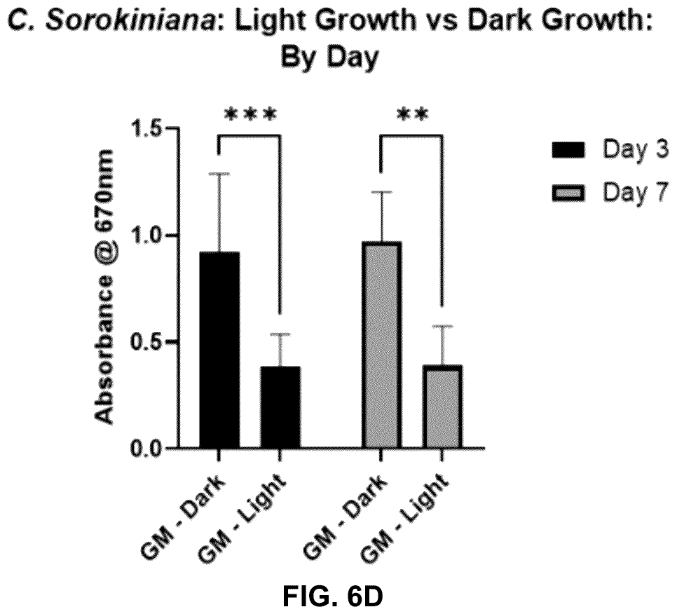

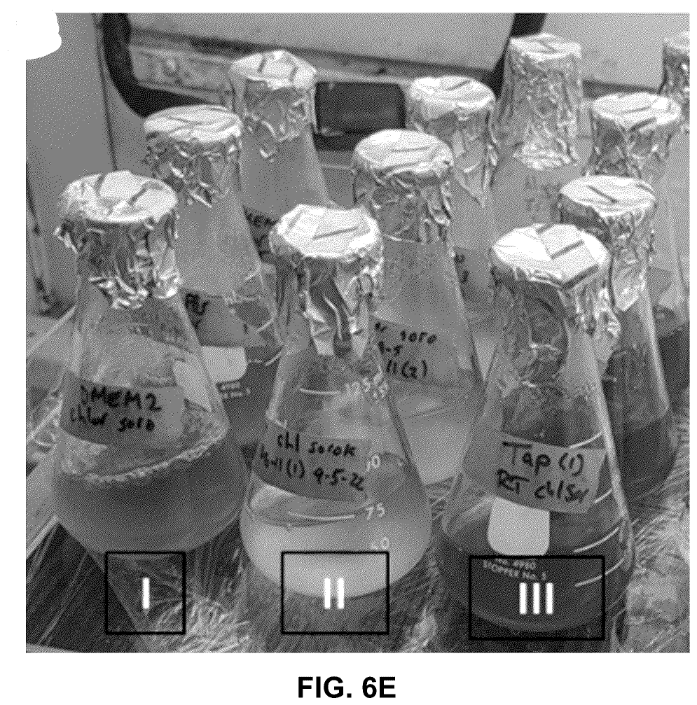

FIG. 6A shows growth of C. sorokiniana at different light intensities. In FIG. 6A, the algal growth curves are plotted when exposed to light intensities of 13, 26, 64, and 165 μmol/m²/s of light. FIG. 6B shows C. sorokiniana absorbance readings when exposed to light intensities of 13, 26, 64, and 165 μmol/m²/s of light, represented by day. Each group experiencing variable light intensity is compared to all other groups at that time point. FIG. 6C shows C. sorokiniana growth curves in GM when grown in the light (60±5 μmol/m²/s) and in the dark. FIG. 6D shows algal growth in the light (60±5 μmol/m²/s) vs. in the dark for 3 and 7 days. One, two, and three asterisks denote a p≤0.05, 0.01, and 0.001, respectively. n≥5. FIG. 6E shows a photo of flask cultures of C. sorokiniana in GM (I), BG-11 (II) and TAP (III) grown for 7 days at 52±5 μmol/m²/s.

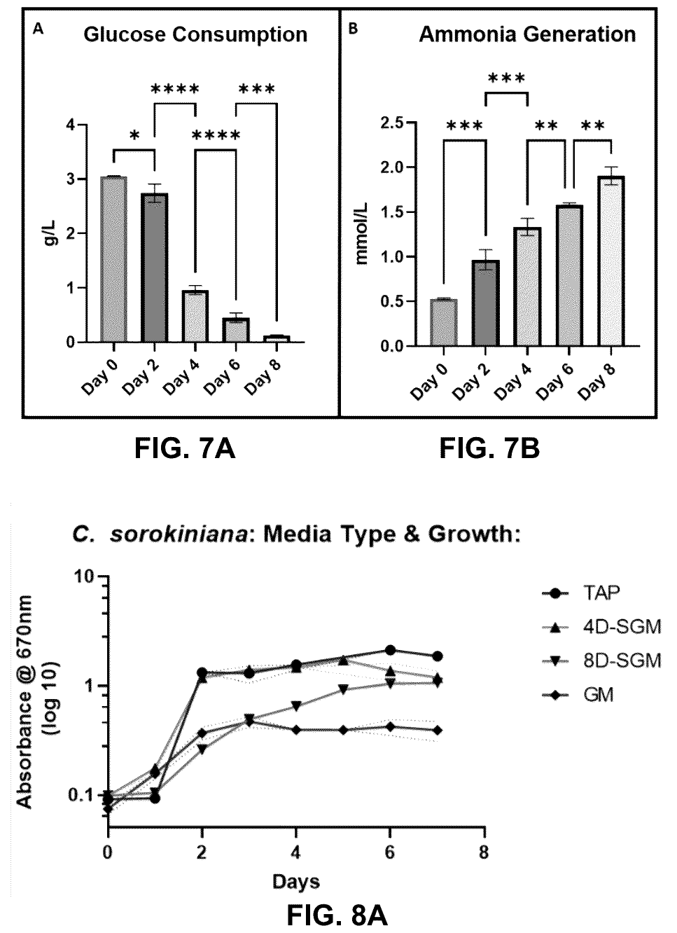

3.2 Spent Media Analysis: Total glucose and ammonia concentrations were measured over 8 days of QM7 cell growth. After 4 days, the total concentration of glucose in the media decreased from 3 to 0.95±0.08 g/L, and by day 8 it was 0.12 g/L (FIG. 7A). Simultaneously, the total concentration of ammonia in the media increased from 0.5 at day 0 to 1.90±0.10 mmol/L by day 8 (FIG. 7B). FIG. 7A shows a plot of glucose consumption. FIG. 7B shows a plot of ammonia generation. In FIG. 7A and in FIG. 7B, Total glucose (g/L) and ammonia (mmol/L) levels in GM after 8 days of QM7 growth. One, two, three, and four asterisks denote a p≤0.05, 0.01, 0.001 and 0.0001, respectively; and n=5.

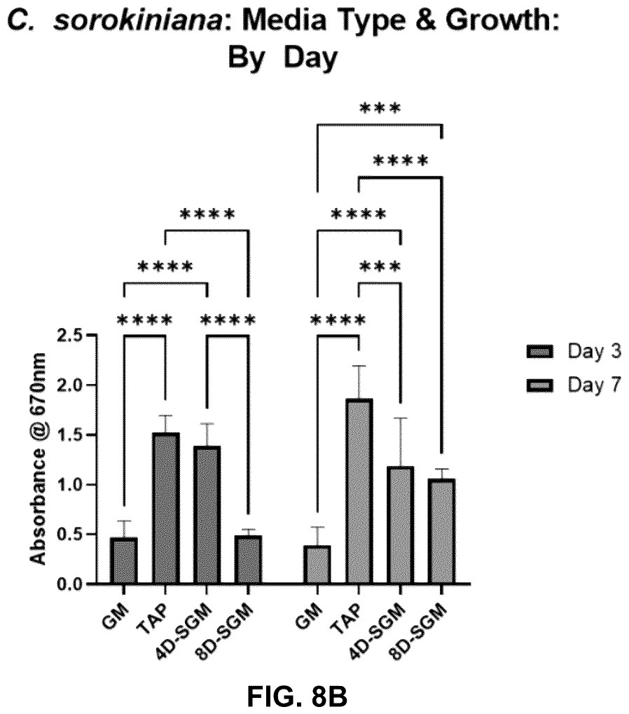

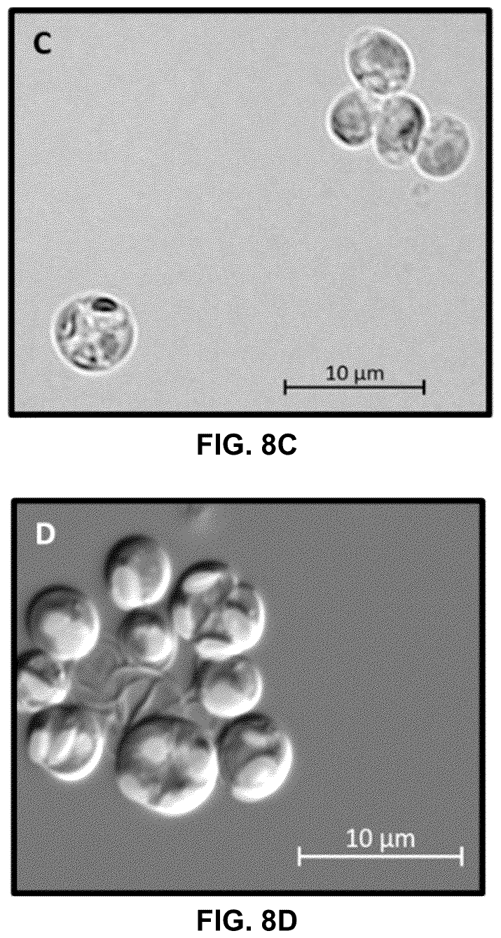

3.3 C. sorokiniana Growth in Spent Media: C. sorokiniana was inoculated into TAP media, GM, 4D-SGM, 8D-SGM, and BG-11 media and grown for 7 days at 37° C. at 60±5 μmol/m²/s. Brightfield images showed that the algae appeared normal with intact cell walls and characteristic green chloroplasts, and, with fluorescent overlay, exhibited chlorophyll autofluorescence after 7 days of growth in animal cell GM (FIG. 8A, FIG. 8B). After 3 days, growth was observed in all groups (FIG. 3C). Compared to GM at day 3 there was about 3-fold more growth in the TAP and 4D-SGM grown cells. Growth in TAP and 4D-SGM continued to day 6. After day 6, cell growth in 4D-SGM began to decline while in TAP it continued to increase. Although algal growth in 8D-SGM reached the same yield at day 7 as cells growing in 4D-SGM, the growth kinetics were substantially different. Algal growth rates and yields were significantly greater for cells in TAP, 4D-SGM and 8D-SGM than in GM (FIG. 3D) over 7 days. FIG. 8A shows a plot of C. sorokiniana media type and growth. FIG. 8B shows a graph of C. sorokiniana media type and growth by day. FIG. 8C shows a microscopic brightfield image of C. sorokiniana after 7 days growth in GM. FIG. 8D shows an autofluorescence of algal chlorophyll after 7 days growth in GM 649 nm/667 nm excitation/emission. The algal growth kinetics in different media formulations and conditions were compared. In FIG. 8B, C. sorokiniana absorbance readings at days 3 and 7 when grown in GM, TAP, 4D-SGM, 8D-SGM, and BG-11. Each group is compared to GM at that time point. One, two, three, and four asterisks denote a p≤0.05, 0.01, 0.001 and 0.0001, respectively; and n≥5.

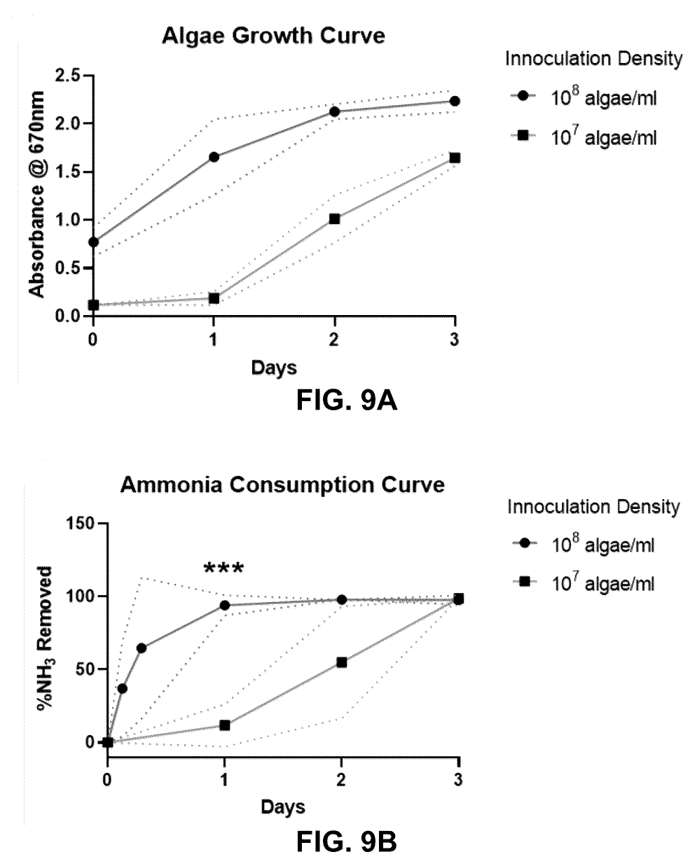

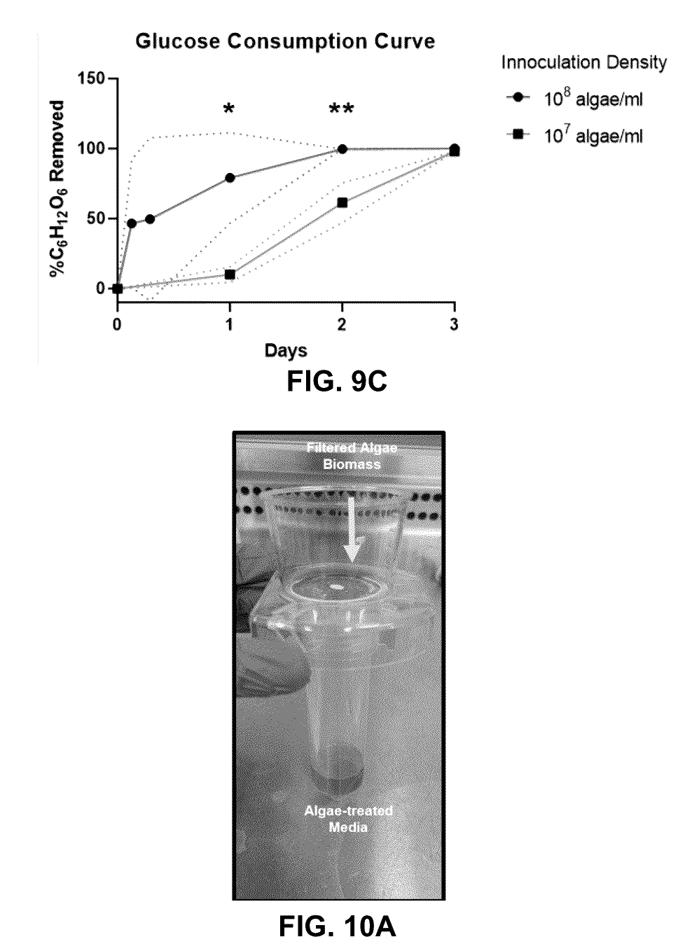

3.4 C. sorokiniana Consumption of Ammonia and Glucose: Algae were inoculated into 4D-SGM at two cell densities and grown for 3 days (FIG. 9A). A lag period spanning ˜1 day was observed for cultures inoculated at the lower density, 10⁷ cells/mL. After one day, ammonia and glucose decreased in each inoculation group, with a significantly greater percentage being removed by the 10⁸ cells/mL group (79.17±32.31% ammonia and 94.13±6.96% glucose) compared to the 10⁷ group (11.84±13.09% ammonia and 10.26±5.1% glucose) (FIG. 9B and FIG. 9C). The rate of consumption for both media components was greater in the 10⁸ group, but by day 3 both groups had metabolized all of the glucose and ammonia in the media.

FIG. 9A shows an algae growth curve. FIG. 9B shows an ammonia consumption curve. FIG. 9C shows a glucose consumption curve. Glucose and ammonia consumption by algae in 4D-SGM media at 2 inoculation densities was measured. In FIG. 9A, C. sorokiniana growth is shown when inoculated into 4D-SGM at densities of 108 and 107 algal cells/mL. In FIG. 9B, percent ammonia removed from 4D-SGM over time at inoculation densities of 10⁸ and 10⁷ cells/mL is shown. In FIG. 9C, percent glucose removed from 4D-SGM over time at inoculation densities of 10⁸ and 10⁷ cells/mL is shown. One, two, and three, asterisks denote a p≤0.05, 0.01, and 0.001 respectively; and n≥5.

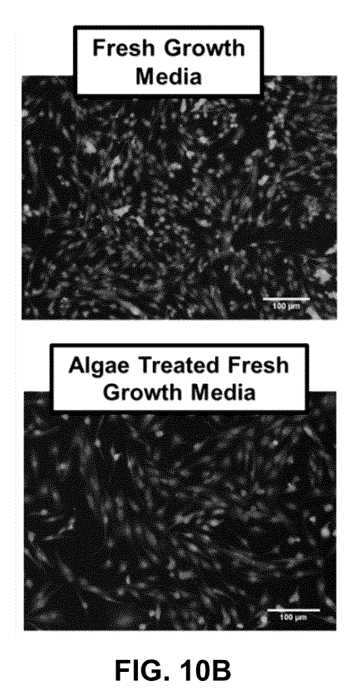

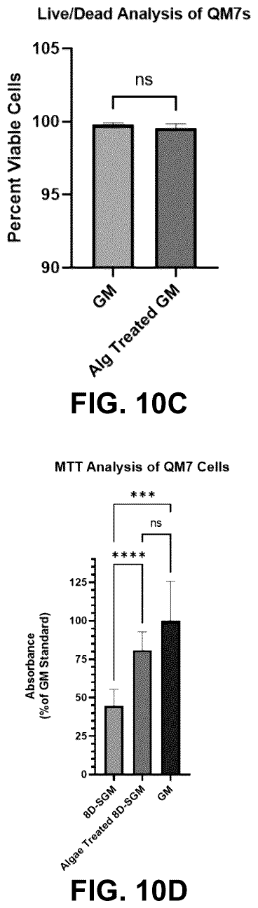

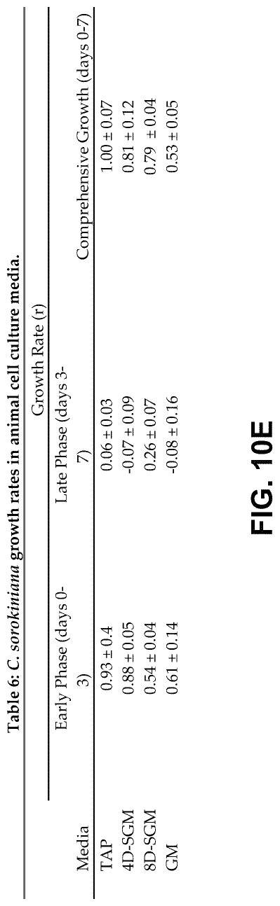

3.5 Response of QM7 Cells to Algal Treated Media: For qualitative observation, C. sorokiniana was grown in phenol red containing GM. After algae were filtered from media, the algal conditioned media appeared pink, as expected of fresh GM media containing phenol red, indicating no substantial change in pH (FIG. 10A). For quantifying the cytotoxicity of algal-treated GM, C. sorokiniana was grown in phenol-red-free GM, and QM7 cells were cultured in the algal treated fresh GM. Live/dead analyses were performed, and there was no statistically significant difference between the fresh GM and the algal treated-GM group (FIG. 10B, FIG. 10C).

When the metabolic activity of QM7 cells was compared after 1 day of culture in GM, 8D-SGM, and Algal-treated-8D-SGM media, MTT assay showed there was no significant difference between GM and Algal-treated-8D-SGM grown cells. In contrast, QM7 cells grown in 8D-SGM media exhibited significantly less metabolic activity (FIG. 10D).

QM7 cell response to algal-treated media is illustrated in FIG. 10A, FIG. 10B, FIG. 10C, and FIG. 10D. FIG. 10A shows C. sorokiniana grown in GM containing phenol red passing through a vacuum filter. As the media passes through the filter a color change from dark red to pink is observed and a bright green cake of algae biomass is retained at the filter. FIG. 10B shows QM7 cells stained using Live (green cytoplasm)/dead (red nuclei) stain. All nuclei are also stained with Hoecsht 33342 (Blue). FIG. 10C shows a graph of cell viability, determined via colocalization of dead signal with nuclear stain, is compared between QM7 cells cultured in GM and 8D-SGM after four days. FIG. 10D shows an MTT Analysis of QM7 cells that were exposed to GM, 8D-SGM, and Alg-Treated-8D-SGM. Absorbance data for each condition is represented as the percentage of the average absorbance recorded for the fresh media group for its corresponding MTT test.

### 4. Discussion

Advances in bioprocessing have enabled the affordable production of many commercial products in the food and pharmaceutical industries¹⁴⁸. Recognizable food products such as yeast, vinegar, and yogurt, as well as thickeners and preservatives such as xanthan gum and lactic acid, respectively, have been mass produced in well controlled processes in compliance with regulatory safety requirements for decades¹⁴⁹⁻¹⁵³. Adjacently, many complex molecules and modern medicines including antibiotics, enzymes, and vaccines, have been affordably mass produced and globally distributed; however, affordable mass production of next generation foods and medicines, including the products of cellular agriculture, and cell therapies is possible but is still technically and economically limited^{148,154}. If such next generation technologies are to become available and affordable to large populations, novel strategies for affordable production must be developed. In this study we explored the ability of a small green alga, C. sorokiniana, to grow mutualistically with animal cells for improved bioprocessing of lab-grown meat.

The accumulation of ammonia in cell culture media is primarily driven by the metabolism of glutamine and other amino acids by animal cells^{129,155,156}. Glutamine is both a nitrogen source for protein synthesis and an alternative to glucose as a carbon energy source. It is supplied as the most concentrated amino acid of many cell culture media formulations including Essential-8, a commonly proposed formulation for rapid growth of cells such as those for cellular agriculture applications, due to its ability to rapidly enter cells and contribute to total protein biomass^{128,133,157}. Ammonia is released when glutamine is converted to glutamate for contribution to ATP production in the Krebs cycle during cell growth. Deamidation of glutamine, yielding ammonia, also spontaneously occurs during its lifespan in a bioreactor, independent of living cells, at rates dependent on its temperature and pH¹⁵⁸. Ultimately, ammonia's rapid accumulation is expected in rapid expansion bioprocesses which use high concentrations of glutamine.

Previous studies explore, with mixed results, the effects of the reduction or replacement of glutamine with other compounds for animal cell cultures on cell productivity. For example, complete elimination of glutamine in batch culture of HEK293-D9 resulted in approximately 50% reduction of the integral of viable cell density. Another glutamine replacement, L-alanyl-I-glutamine (commercially marketed as Glutamax), has repeatedly demonstrated support of animal cell growth with reduced accumulation of media; however, in one study, the growth rate of the target Chinese hamster ovarian cell line was reduced during the first seven days of culture¹⁵⁹. After day 7, in glutamine containing cultures, concentrations of ammonia that had previously been observed to be detrimental to the strain's growth were recorded alongside a greater apoptotic ratio. Together, these results suggest that cultures that include glutamine, alongside a strategy to eliminate the ammonia before it accumulates beyond a detrimental threshold, may exhibit faster growth without the deleterious effects of the ammonia.

Several strategies to eliminate ammonia waste from animal cell culture media for pharmaceutical applications have been established; however, they have not yet been widely adapted for the context of cultured meat. Materials like zeolite, zirconium phosphate, activated carbon and anionic resins, can be packed into columns and introduced into bioreactors to selectively adsorb ammonia from the aqueous solutions¹⁶⁰⁻¹⁶³. The columns are subsequently removed from the media and then stripped of the adsorbed ammonia before reuse. Gas-permeable membranes made from materials such as polyvinylidene fluoride have also been used to encourage passive diffusion of ammonia from aqueous solutions, such as spent cell culture media, to a permeate side containing an acid which would then be disposed of as waste^{164,165}. Another method, electrodialysis, or the application of a DC electric field to the culture media has been incorporated into systems for selectively removing ammonia¹⁶⁶. These technologies all have successfully demonstrated the removal of ammonia for the preservation of media integrity; however, a recent analysis suggests that without a strategy for reclamation and reuse of waste nitrogen from growth media, cultured meat produced at scale would still have nearly as much nitrogen waste generated as a percentage of nitrogen input into the system (76%) as conventional beef systems (84%), and even more nitrogen waste than swine (47%) or poultry (55%) systems¹⁶⁷.

A novel approach to nitrogen removal and reclamation, first developed by Haraguchi, et al., described as a circular cell culture system that employs microalgae for synthesizing proteinogenic amino acids from waste ammonia before returning them to the cell culture, may be an ideal system for sustainable cultured meat production^{137,168}. Microalgae such as freshwater C. vulgaris and marine C. littorale were both investigated as potential biocatalyst species for reclaiming nitrogen from spent media. While both species demonstrated the ability to eliminate ammonia from the media waste, algal cultivation studies were performed at 30° C., rather than 37° C. at which animal cells would typically be cultured. For this reason, we explored the capability of a thermoresistant strain of algae, C. sorokiniana, as an alternative biocatalyst.

A number of variables define the growth capabilities and metabolism of microalgae and, depending on the geographical environment and habitats within which the species have adapted. Adaptive laboratory evolution, or the intentional induction of stress in a controlled environment, can also modify the behavior of algae under unique growth conditions¹⁶⁹. For example, light intensity can affect species that may be shaded by canopy or caves, or exposed to direct sunlight in tropical deserts¹⁷⁰. We explored the effect of light intensity on the growth of C. sorokiniana in animal cell culture medium to identify lighting conditions that would be satisfactory for algal growth in an animal cell bioreactor. We observed that C. sorokiniana grown under the highest light intensity exhibited the least amount of growth. Due to the complex relationship between light intensity and quality, CO₂ consumption, exogenous carbon, and photosynthesis, it is difficult to identify the root cause of the reduction in growth¹⁷¹. It is possible that high light intensities led to rapid CO₂ consumption and CO₂ sooner becoming a limiting component to photosynthesis. We suggest future studies exploring the relationship between light intensity and CO₂ consumption rates. Additionally, while the maximum intensity used in this study, 164 μmol/m²/sec, did not approach that found in some outdoor cultivation plants (˜2000 μmol/m²/sec at noon), it is possible that the high intensity of the light caused photoinhibition in the culture and thus limited its growth¹⁷². Photoinhibition, or the reduction in photosynthetic capability in the presence of too much light, is marked with a reduction in CO₂ assimilation by the organism and is believed to primarily be the result of damage to Photosystem II ultimately limiting growth¹⁷³.

C. sorokiniana also exhibit the metabolic plasticity for not just photoautotrophic growth (light dependent), but also heterotrophic growth where growth occurs in the absence of light¹⁴⁴. Instead, carbon sources, such as glucose or acetate, are assimilated from the media for metabolism. Additionally, previous studies demonstrate the capacity for C. sorokiniana and other species to exhibit mixotrophic growth, which refers to the simultaneous use of photoautotrophic and heterotrophic metabolic strategies^{136,144,174}. Because of the presence of glucose in GM, it is important to note that the addition of light to C. sorokiniana cultures in GM may modify the metabolic strategy of the algae from heterotrophic to mixotrophic. The advantages of heterotrophic and mixotrophic cultivation over photoautotrophic cultivation for improved growth rates, biomass yield, and enhanced material quality and are well documented within the context of biofuel manufacturing^{175,176}. We explored the growth of C. sorokiniana in GM when exposed to light and found that, after three days, C. sorokiniana maintained in the dark exhibited significantly more growth than when grown in light. Previous studies describe, for photoautotrophic species, a phenomenon referred to as self-shading, which is characterized as the reduction in algal specific growth rate due to light obfuscation by the algal biomass itself, can limit further algal growth^{177,178}. Additionally, photoautotrophic species are dependent on the presence of CO₂ for photosynthesis; thus, to prevent growth limitations, CO₂ is typically sparged into photobioreactors at additional production cost¹⁷⁹. Our results suggest that the design and implementation of a heterotrophic C. sorokiniana driven cell culture media recycling system may not depend on balancing the complex kinetics of the algal demand for both light and CO₂¹⁸⁰.

To define metrics that assess the productivity of animal cell bioprocesses, one must consider the desired output of the culture^{148,181}. For pharmaceutical applications this could be, for example, the maximum amount of cells grown per unit volume of media, referred to as cell yield, or, e.g., the mass of antibody produced per liter per day¹⁸². For cellular agriculture, this could be either the cell density or the percentage of successfully differentiated myocytes¹⁹. It is the goal of bioprocess engineers to ensure that their subject cells exist in their most metabolically efficient state that maximizes a predefined productivity metric. Thus, it is important to assess the consumption rates of metabolic fuel, such as glucose, and the production rates of metabolic waste, such as ammonia, for each unique cell type and the cells' respective metabolic goals. Understanding that C. sorokiniana has the capacity to grow mixotrophically, and that algal growth rate is accelerated in media containing ammonia¹⁴⁰, it is important to monitor, for the sake of algal growth management, the rates at which animal cells consume and produce these critical components. We found that glucose consumption and ammonia generation by QM7 cells was continuous across 8 days of culture. After 8 days, the concentration of ammonia had accumulated beyond 1.8 mmol, a common threshold at which point ammonia inhibits cell growth, and glucose was nearly exhausted¹⁸³. The greatest increase in ammonia occurred between days 6 and 8 of culture. This may be due to the cells increasing their dependence on glutamine metabolism for ATP generation due to the lower concentrations of glucose.

Because C. sorokiniana grows faster heterotrophically, we hypothesized that algal growth in 4D-SGM would be greater than in 8D-SGM, due to the concentrations of glucose in the media. Simultaneously, we hypothesized faster growth in 4D-SGM and 8D-SCG than in GM, due to the presence of higher concentrations of ammonia. C. sorokiniana was inoculated into each media group and, over time, notable differences in growth rate were observed (Table 1).

In GM, which contained the highest concentration of glucose but the lowest concentration of ammonia, growth was observed to day 3. This growth was comparable to 8D-SGM, which contained little glucose, but had the most ammonia. Beyond day 3, growth continued in 8D-SGM while growth declined in GM. These results suggest that higher concentrations of glucose, upon C. sorokiniana inoculation, do not necessarily ensure longer term (>3 days) growth. The presence of ammonia and light may enable longer-term growth in animal cell media than in GM, despite less initial glucose. C. sorokiniana that was inoculated into 4D-SGM contained significantly more glucose and less ammonia than 8D-SGM and significantly less glucose and more ammonia than GM, yet after 3 days, growth was comparable to that in TAP media, and there was twice the growth in each other group. Those results suggested that the combination of both ammonia and glucose may have driven greater immediate growth in animal cell culture media than the presence of ammonia or glucose alone. While the density of C. sorokiniana in 4D-SGM remained higher, its density declined after day 3 in contrast to the 8D-SGM, which showed continued growth throughout the extent of the experiment. Ultimately, these data suggest that, in animal cell culture media, C. sorokiniana exhibits complex growth kinetics that are dependent on the number of days in which QM7 cells are cultured. FIG. 10E shows Table 6: C. sorokiniana growth rates in animal cell culture media.

In FIG. 10E (Table 6), early phase refers to the growth rate calculated during the first three days following inoculation where ODₜ and OD₀ refer to OD recorded at days 3 and 0, respectively, and t is equal to 3 days. Late phase refers to the growth rate between days 3 and 7 where ODₜ and OD₀ refer to OD recorded at days 3 and 7, respectively, and t is equal to 4 days. Comprehensive growth refers to the growth rate calculated for the length of the experiment where ODₜ and OD₀ refer to OD recorded at days 0 and 7, respectively, and t is equal to 7 days.

We explored how the inoculation density of the algae affects the speed at which ammonia and, due to mixotrophic growth, glucose, can be removed from spent media. Within 24 hours, nearly 100% of ammonia was removed from the media in the high-density group and reductions in total ammonia and glucose were observed as early as 3 hours post inoculation for the high-density group (FIG. 9A-C). By day 3, nearly all ammonia and glucose, with little variation, was removed from spent media in both inoculation density groups. These results suggest that bioprocesses can be designed to facilitate rapid removal of ammonia from spent animal cell growth media. These results, at day 3, are consistent with previously reported ammonia consumption data for C. littorale, and surpass those data for C. vulgaris¹⁶⁸.

Having demonstrated the capacity for C. sorokiniana to eliminate ammonia from spent animal cell culture media, it was of importance to ensure that there was limited cytotoxic consequences on animal cells for having grown algae in its growth media. Based on our limited analyses, our results suggest that little to no metabolites were released into the animal cell culture media because of C. sorokiniana growth that imposes short term cytotoxic effects on QM7 cells. Similarly, the metabolic activity of QM7 cells cultured in algal-treated media was examined and after 24 hours of culture, the cumulative metabolic activity of the QM7 cell cultures in algae treated 8D-SGM was greater than that of untreated 8D-SGM, and comparable to that in GM, per MTT assay. Additional studies are needed to determine how algal-treated media affects other key cell behaviors such as animal cell growth rates, cell adhesion, cell motility, and cell differentiation. Such studies can inform bioprocess operations, such as residence time, length of time of algal treatment, and potentially timing of replenishment of key nutrients, such as glucose¹⁴⁸.

Other media constituents include serum or serum replacements, growth factors (such as FGF or TGF-β), inorganic salts (such as magnesium chloride, potassium chloride, and sodium phosphate), amino acids (such as L-glutamine, L-lysine, L-arginine, etc.), and vitamins¹⁸⁴⁻¹⁸⁷. All play important roles in supporting key processes in cell growth, metabolism, and differentiation, so future research should explore the potential reduction additional animal cell media components following microalgal growth. Having demonstrated that C. sorokiniana has the ability to grow under the same environmental conditions as animal cells, we suggest the investigation of novel semipermeable membrane technology for separation of animal and algal cells within the same bioproduction unit, so that continuous operation could be maintained in a mutualistically symbiotic ecosystem inspired bioreactor¹⁸⁸.

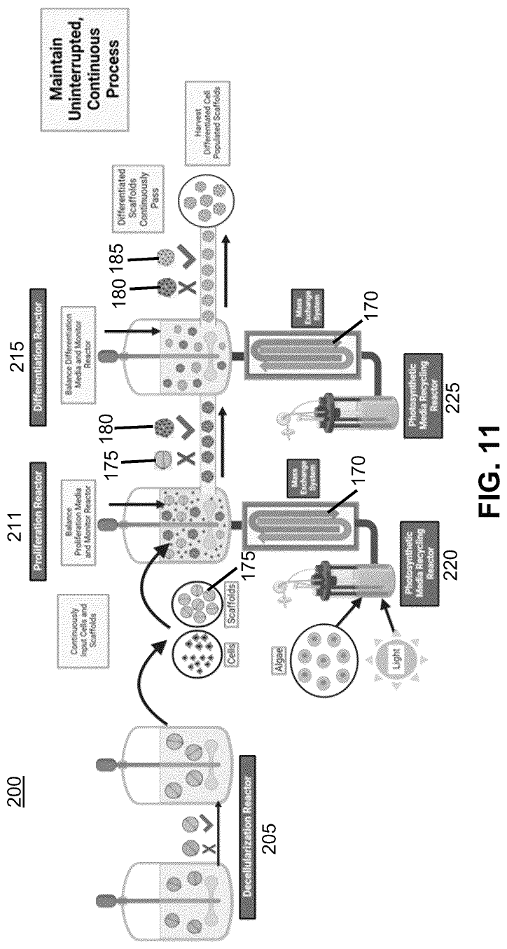

A proposed bioprocess²⁰⁰ (FIG. 11), could involve preparation of affordable decellularized scaffolds from plants or agricultural waste for inoculation, with animal cells, into a proliferation reactor^{146,189,190}. Within this proliferation reactor, cell culture media designed specifically to support proliferation would be provided and continuously monitored. As the media is metabolized by the animal cells, it would be continuously pumped through a mass exchange system 170 designed to enable media to enter a secondary microalgal reactor without any cross transfer of animal or algal cells. Within this microalgal reactor, C. sorokiniana should continuously eliminate ammonia before the animal culture medium is reintroduced into the proliferation reactor via the mass exchange system 170. As animal cells continue to grow in the proliferation reactor, confluent cell carriers are passed into a differentiation reactor which would be similarly supported by its own microalgal reactor. FIG. 11 shows a proposed bioprocess. The bioprocess in FIG. 11 is including a decellularization reactor 205, a proliferation reactor 211, a differentiation reactor 215, a first photosynthetic media recycling reactor 220, and a second photosynthetic media recycling reactor 225. The bioprocess in FIG. 11 can maintain an uninterrupted, continuous process. Two or more different cells can grow in an adherent growth upon the mammalian cell carrier 175 in the proliferation reactor 211 to produce a differentiated growth seed 180 that is passed into the differentiation reactor 215 and grown into a differentiated scaffold 185. The differentiated scaffold 185 can then be continuously harvested as a differentiated cell populated scaffold.

### 5. Conclusions

The availability and affordability of next generation medicines, such as cell therapies, and cellular agriculture products, such as cultured meat, will depend on the implementation of affordable bioproduction strategies. We demonstrate the ability for C. sorokiniana, a freshwater alga, to grow in spent animal cell culture media at 37° C., and rapidly sequester ammonia. We observed microalgal growth behavior is dependent on the presence and intensity of light, and the presence of ammonia and glucose. We also found no cytotoxic effects of C. sorokiniana growth in cell culture media and the potential to extend the lifespan of spent media, as indicated by QM7 metabolic activity. These results justify further investigation into the capacity of C. sorokiniana to reduce cell culture media waste and the efficiency of such a process. We posit that the integration of algal bioreactors into animal cell production lines may enable development of new resource-efficient, cost-effective, semicontinuous manufacturing systems that enable affordable mass production of animal cell populations.

### Description of Embodiments

This is a novel invention that not only supports the growth of algae in mammalian cell growth media but is able to do this at a pH of 7.4 and at a temperature of 37° C., which is critical for incorporation into existing bioprocesses at relevant cell culture temperature and pH. This removes the need to cool down the media and then heat it back up during algae treatment. This is due, in some embodiments, to the use of an uncommon thermophilic algae strain.

In accordance with some embodiments, a method is provided for producing a biological product. The method involves obtaining a biological cell production system that includes a living biological cell culture and an exchange system. The living biological cell culture produces biological waste, nutrients, oxygen, or carbon dioxide. The exchange system allows a biological growth medium to enter and exit the living biological cell culture. The biological growth medium is circulated through the exchange system, and the biological product is produced by the growth of the living biological cell culture.

In accordance with other embodiments, a biological cell production system is provided for producing a biological product. The system includes a living biological cell culture capable of producing biological waste, nutrients, oxygen, or carbon dioxide, an exchange system capable of allowing a biological growth medium to enter and exit the living biological cell culture, and a mechanism for circulating the biological growth medium through the exchange system. The biological product is produced by the growth of the living biological cell culture.

In some embodiments, the techniques described herein relate to a mammalian cell production system, including: A) a living mammalian cell culture including a mammalian growth medium operative to feed nutrients and oxygen to a plurality of mammalian cells within the mammalian cell culture and to transport a mammalian waste from the plurality of mammalian cells; B) a living algae cell culture including an algae growth medium operative to feed nutrients and carbon dioxide to a plurality of algae cells within the algae cell culture and to transport an algae waste from the plurality of algae cells; wherein the living algae cell culture produces the algae waste with one or more nutrients and oxygen operative to feed the mammalian cell culture and wherein the living mammalian cell culture produces the mammalian waste with one or more nutrients and carbon dioxide operative to feed the algae cell culture; and C) a mass exchange system operative to allow the mammalian growth medium to enter and/or to exit the living algae cell culture and to allow the algae growth medium to enter and/or to exit the living mammalian cell culture without a cross transfer of mammalian cells and algae cells; wherein the system is capable of a continuous operation at a temperature in a range from about 30° C. to about 40° C. and a pH in a range from about 7 to about 8 thereby removing a need to cool down the algae growth medium and/or heat up the algae growth medium for a treatment of algae or for a replenishment of algae.

According to some aspects, the techniques described herein relate to a mammalian cell production system, wherein the system is capable of a continuous operation at a temperature at about 37° C.

In some aspects, the techniques described herein relate to a mammalian cell production system, wherein the system is capable of a continuous operation at a pH at about 7.4.

In some embodiments, the techniques described herein relate to a mammalian cell production system, wherein the living algae cell culture is capable of receiving a light energy that is directed upon the plurality of algae cells.

According to some aspects, the techniques described herein relate to a mammalian cell production system, wherein the mammalian waste includes urea, carbon dioxide, lactic acid, ammonia, or a combination thereof.

According to some aspects of the technology herein, the techniques described herein relate to a mammalian cell production system, wherein the algae waste includes glucose, oxygen, or a combination thereof.

In some embodiments, the techniques described herein relate to a mammalian cell production system, further including a mammalian cell substrate or a mammalian cell carrier operative to provide an adherent growth substrate for a plurality of mammalian cells.

According to some aspects, the techniques described herein relate to a mammalian cell production system, further including the plurality of mammalian cells include two or more mammalian cell types, and wherein the living mammalian cell culture is within a proliferation reactor that is in a fluid communication with the mass exchange system and with a conduit leading into a filter and a differentiation reactor; wherein the filter provides a passage of a differentiated growth seed including an adherent growth of the two or more cell types from the proliferation reactor and into the differentiation reactor.

In some aspects, the techniques described herein relate to a mammalian cell production system, further including the differentiation reactor is in fluid communication with the mass exchange system; and the differentiation reactor provides a chamber for the differentiated growth see to grow into a differentiated cell populated scaffold; and the differentiation reactor includes an outlet operative to provide the differentiated cell populated scaffold.

In some embodiments, the techniques described herein relate to a mammalian cell production system, further including the mammalian cells include and/or are replaced by animal cells and/or vertebrate cells.

According to some aspects, the techniques described herein relate to a mammalian cell production system, further including the algae cells include and/or are replaced by one or more cells from a microorganism.

According to some aspects of the technology herein, the techniques described herein relate to a mammalian cell production system, further including one or more pumps, each of the one or more pumps capable of moving the mammalian growth medium and/or the algae growth medium.

In some embodiments, the techniques described herein relate to a mammalian cell production system, wherein one or more algae cells include Chlorella sorokiniana, Chlorococcum littorale, Chlorella vulgaris, Chlamydomonas reinhardtii, Parachlorella, Desmodesmus armatus, or a combination thereof.

According to some aspects, the techniques described herein relate to a mammalian cell production system, wherein one or more mammalian cells include stem cells, human cells, ruminants' cells, murine cells, porcine cells, bird cells, or a combination thereof.

In some aspects, the techniques described herein relate to a mammalian cell production system, wherein the system is capable of a production of a mammal tissue suitable for a human consumption under one or more standards of human consumption.

In some embodiments, the techniques described herein relate to a mammalian cell production system, wherein the production of a mammal tissue is a meat production; wherein the meat is suitable for human consumption; and wherein the meat production is provided with a lower waste emission into an environment when compared to a production of the same meat utilizing one or more living animals.

According to some aspects, the techniques described herein relate to a mammalian cell production system, wherein the mass exchange system includes a semi-permeable membrane, a filter, a zeolite, a hollow fiber, a filter, or a combination thereof.

According to some aspects of the technology herein, the techniques described herein relate to a method for producing a tissue product, the method including the steps of: (1) obtaining a mammalian cell production system, including: A) a living mammalian cell culture including a mammalian growth medium operative to feed nutrients and oxygen to a plurality of mammalian cells within the mammalian cell culture and to transport a mammalian waste from the plurality of mammalian cells; B) a living algae cell culture including an algae growth medium operative to feed nutrients and carbon dioxide to a plurality of algae cells within the algae cell culture and to transport an algae waste from the plurality of algae cells; wherein the living algae cell culture produces the algae waste with one or more nutrients and oxygen operative to feed the mammalian cell culture and wherein the living mammalian cell culture produces the mammalian waste with one or more nutrients and carbon dioxide operative to feed the algae cell culture; C) a mass exchange system operative to allow the mammalian growth medium to enter and/or to exit the living algae cell culture and to allow the algae growth medium to enter and/or to exit the living mammalian cell culture without a cross transfer of mammalian cells and algae cells; (2) circulating the mammalian growth medium and the algae growth medium, whereby the mammalian growth medium and the algae growth medium enter and exit the mass exchange system; and whereby the tissue product is produced by a growth of the living mammalian cell culture that is at least partially sustained by the algae cell culture.

In some embodiments, the techniques described herein relate to a method, wherein the system is capable of a continuous operation at a temperature in a range from about 30° C. to about 40° C. and a pH in a range from about 7 to about 8 thereby removing a need to cool down the algae growth medium and/or heat up the algae growth medium for a treatment of algae or for a replenishment of algae.

According to some aspects, the techniques described herein relate to a method, wherein the system is capable of a continuous operation at a temperature at about 37° C.

In some aspects, the techniques described herein relate to a method, wherein the system is capable of a continuous operation at a pH at about 7.4.

In some embodiments, the techniques described herein relate to a method, further including a mammalian cell substrate or a mammalian cell carrier operative to provide an adherent growth substrate for a plurality of mammalian cells.

According to some aspects, the techniques described herein relate to a method, further including the plurality of mammalian cells include two or more mammalian cell types, and wherein the living mammalian cell culture is within a proliferation reactor that is in a fluid communication with the mass exchange system and with a conduit leading into a filter and a differentiation reactor; wherein the filter provides a passage of a differentiated growth seed including an adherent growth of the two or more cell types from the proliferation reactor and into the differentiation reactor.

According to some aspects of the technology herein, the techniques described herein relate to a method, further including the differentiation reactor is in fluid communication with the mass exchange system; and the differentiation reactor provides a chamber for the differentiated growth see to grow into a differentiated cell populated scaffold; and the differentiation reactor includes an outlet operative to provide the differentiated cell populated scaffold.

In some embodiments, the techniques described herein relate to a method, wherein the living algae cell culture is capable of receiving a light energy that is directed upon the plurality of algae cells.

According to some aspects, the techniques described herein relate to a method, wherein the mammalian waste includes urea, carbon dioxide, lactic acid, ammonia, or a combination thereof.

In some aspects, the techniques described herein relate to a method, wherein the algae waste includes glucose, oxygen, or a combination thereof.

In some embodiments, the techniques described herein relate to a method, further including the mammalian cells include and/or are replaced by animal cells and/or vertebrate cells.

According to some aspects, the techniques described herein relate to a method, further including the algae cells include and/or are replaced by one or more cells from a microorganism.

According to some aspects of the technology herein, the techniques described herein relate to a method, further including one or more pumps, each of the one or more pumps capable of moving the mammalian growth medium and/or the algae growth medium.

In some embodiments, the techniques described herein relate to a method, wherein one or more algae cells include Chlorella sorokiniana, Chlorococcum littorale, Chlorella vulgaris, Chlamydomonas reinhardtii, Parachlorella, Desmodesmus armatus, or a combination thereof.

According to some aspects, the techniques described herein relate to a method, wherein one or more mammalian cells include stem cells, human cells, ruminants' cells, murine cells, porcine cells, bird cells, or a combination thereof.

In some aspects, the techniques described herein relate to a method, wherein the system is capable of a production of a mammal tissue suitable for a human consumption under one or more standards of human consumption.

In some embodiments, the techniques described herein relate to a method, wherein the production of a mammal tissue is a meat production; wherein the meat is suitable for human consumption; and wherein the meat production is provided with a lower waste emission into an environment when compared to a production of the same meat utilizing one or more living animals.

According to some aspects, the techniques described herein relate to a method, wherein the mass exchange system includes a semi-permeable membrane, a filter, a zeolite, a hollow fiber, a filter, or a combination thereof.

According to some aspects of the technology herein, the techniques described herein relate to a method for recycling a mammalian cell culture medium, the method including the steps of: (1) obtaining a mammalian cell production system, including: A) a living mammalian cell culture including a mammalian growth medium operative to feed nutrients and oxygen to a plurality of mammalian cells within the mammalian cell culture and to transport a mammalian waste from the plurality of mammalian cells; B) a living algae cell culture operative to utilize the mammalian growth medium to feed nutrients and carbon dioxide to a plurality of algae cells within the algae cell culture and to transport an algae waste from the plurality of algae cells; wherein the living algae cell culture produces the algae waste with one or more nutrients and oxygen operative to feed the mammalian cell culture and wherein the living mammalian cell culture produces the mammalian waste with one or more nutrients and carbon dioxide operative to feed the algae cell culture; wherein the system is capable of a continuous operation at a temperature in a range from about 30° C. to about 40° C. and a pH in a range from about 7 to about 8 thereby removing a need to cool down the algae growth medium and/or heat up the algae growth medium for a treatment of algae or for a replenishment of algae; (2) extracting the algae waste from the plurality of algae cells; and whereby a recycled mammalian cell culture medium is obtained from the algae waste.

In some embodiments, the techniques described herein relate to a method, further including the step of: (1A) obtaining a mass exchange system operative to allow the mammalian growth medium to enter and/or to exit the living algae cell culture and to allow an algae growth medium to enter and/or to exit the living mammalian cell culture without a cross transfer of mammalian cells and algae cells.

According to some aspects, the techniques described herein relate to a method, wherein the system is capable of a continuous operation at a temperature at about 37° C.

In some aspects, the techniques described herein relate to a method, wherein the system is capable of a continuous operation at a pH at about 7.4.

In some embodiments, the techniques described herein relate to a method, wherein the living algae cell culture is capable of receiving a light energy that is directed upon the plurality of algae cells.

According to some aspects, the techniques described herein relate to a method, wherein the mammalian waste includes urea, carbon dioxide, lactic acid, ammonia, or a combination thereof.

According to some aspects of the technology herein, the techniques described herein relate to a method, wherein the algae waste includes glucose, oxygen, or a combination thereof.

In some embodiments, the techniques described herein relate to a method, further including a mammalian cell substrate or a mammalian cell carrier operative to provide an adherent growth substrate for a plurality of mammalian cells.

According to some aspects, the techniques described herein relate to a method, further including the plurality of mammalian cells include two or more mammalian cell types, and wherein the living mammalian cell culture is within a proliferation reactor that is in a fluid communication with the mass exchange system and with a conduit leading into a filter and a differentiation reactor; wherein the filter provides a passage of a differentiated growth seed including an adherent growth of the two or more cell types from the proliferation reactor and into the differentiation reactor.

In some aspects, the techniques described herein relate to a method, further including the differentiation reactor is in fluid communication with the mass exchange system; and the differentiation reactor provides a chamber for the differentiated growth see to grow into a differentiated cell populated scaffold; and the differentiation reactor includes an outlet operative to provide the differentiated cell populated scaffold.

In some embodiments, the techniques described herein relate to a method, further including the mammalian cells include and/or are replaced by animal cells and/or vertebrate cells.

According to some aspects, the techniques described herein relate to a method, further including the algae cells include and/or are replaced by one or more cells from a microorganism.

According to some aspects, the techniques described herein relate to a method, further including one or more pumps, each of the one or more pumps capable of moving the mammalian growth medium and/or the algae growth medium.

In some embodiments, the techniques described herein relate to a method, wherein one or more algae cells include Chlorella sorokiniana, Chlorococcum littorale, Chlorella vulgaris, Chlamydomonas reinhardtii, Parachlorella, Desmodesmus armatus, or a combination thereof.

According to some aspects of the technology herein, the techniques described herein relate to a method, wherein one or more mammalian cells include stem cells, human cells, ruminants' cells, murine cells, porcine cells, bird cells, or a combination thereof.

According to some aspects, the techniques described herein relate to a method, wherein the system is capable of a production of a mammal tissue suitable for a human consumption under one or more standards of human consumption.

In some embodiments, the techniques described herein relate to a method, wherein the production of a mammal tissue is a meat production; wherein the meat is suitable for human consumption; and wherein the meat production is provided with a lower waste emission into an environment when compared to a production of the same meat utilizing one or more living animals.

In some embodiments, the techniques described herein relate to a method, wherein the mass exchange system includes a semi-permeable membrane, a filter, a zeolite, a hollow fiber, a filter, or a combination thereof.

The field of tissue engineering has seen significant advancements in recent years. This field involves the use of cells, engineering, and materials methods, and suitable biochemical and physiochemical factors to improve or replace biological tissues. One of the key challenges in this field is to provide an optimal environment for cell growth and proliferation. This includes providing the cells with the necessary nutrients and oxygen and removing waste products. Traditionally, this has been achieved through the use of growth mediums. However, these mediums need to be constantly replenished to maintain the optimal conditions for cell growth. This can be a complex and costly process. Additionally, the disposal of waste products from the cell culture can also pose challenges. Therefore, there is a need for improved methods and systems for cell culture and tissue production.

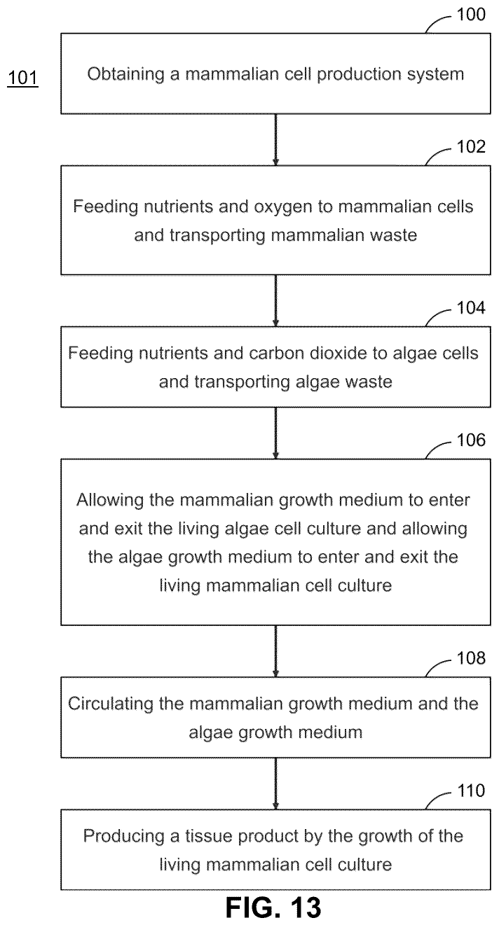

In some embodiments, FIG. 13 shows an example method 101 for a tissue production. The method 101 can be practiced as a meat (or consumable) production method. Step 100 involves the acquisition of a system designed for the cultivation of mammalian cells to produce a biological product. This system includes a mammalian cell culture, which is a group of mammalian cells maintained in a controlled environment to promote growth and metabolism. The system also incorporates an exchange system, which is a mechanism that facilitates the movement of a biological growth medium into and out of the mammalian cell culture. The biological growth medium is a nutrient-rich fluid that supports the life processes of the cells by providing nutrients and removing metabolic waste.

The system is further detailed in sub-step 100-a, which specifies that the system comprises both a mammalian cell culture and an algae cell culture. The algae cell culture is a group of algae cells that perform photosynthesis, producing oxygen and nutrients that can be utilized by the mammalian cells. Conversely, the mammalian cells produce waste containing nutrients and carbon dioxide that can be used by the algae, establishing a symbiotic relationship between the two cultures.

The exchange system mentioned in step 100 is designed to prevent cross-contamination between the mammalian and algae cell cultures while allowing the exchange of growth mediums. This system ensures that the mammalian growth medium can enter and exit the algae cell culture, and the algae growth medium can do the same with the mammalian cell culture, without the two cell types mixing. This exchange is necessary for maintaining the balance of nutrients and waste products between the two cultures, which is essential for the growth and sustainability of the cells.

In summary, step 100 involves setting up a system that includes both mammalian and algae cell cultures, along with an exchange system that manages the flow of growth mediums. This setup is necessary for the production of a tissue product, as it ensures that the mammalian cells receive the necessary support from the algae culture to thrive and produce the desired biological product.

Step 102 involves the process of providing nutrients and oxygen to mammalian cells within a cell culture and the subsequent transportation of waste from these cells. This step is carried out using a growth medium, which is a formulated liquid or gel that supplies necessary nutrients and oxygen to support the metabolic functions of mammalian cells. These functions include growth, division, and energy production through cellular respiration.

The components involved in step 102 are mammalian cells, the growth medium, and a mass exchange system. The mammalian cells consume the nutrients and oxygen from the growth medium to perform their metabolic activities. As a byproduct of these activities, the cells produce waste, which includes substances like carbon dioxide and ammonia.

The growth medium then transports the waste away from the mammalian cells, which is essential to prevent the buildup of potentially harmful byproducts. In sub-step 102-a, the mass exchange system facilitates the transfer of the growth medium, now containing mammalian waste, into the algae cell culture. This waste provides nutrients and oxygen to the algae cells, establishing a symbiotic exchange between the mammalian and algae cell cultures.

The objective of step 102 and sub-step 102-a is to sustain a conducive environment for the growth of mammalian cells while simultaneously supporting the algae cell culture. This exchange between the mammalian and algae cell cultures optimizes the use of resources and aids in waste reduction within the system.

Step 104 involves the provision of nutrients and carbon dioxide to algae cells within an algae cell culture. This step is part of a process that maintains a symbiotic relationship between mammalian and algae cell cultures. The algae cells utilize these inputs to carry out photosynthesis and other metabolic activities necessary for their growth and reproduction. Concurrently, the algae growth medium is circulated to remove waste products generated by the algae cells. This removal is essential to prevent the accumulation of substances that could be harmful to the algae if they were to build up within the culture.

Sub-step 104-a describes the production of waste by the algae cell culture, which contains nutrients and carbon dioxide. This waste serves a functional role in the system, as it provides feed for the mammalian cell culture. The exchange of waste and nutrients between the algae and mammalian cell cultures is facilitated by a mass exchange system, which ensures the proper transfer of these substances without mixing the cell populations.

The process outlined in Step 104 and Sub-step 104-a includes algae cells performing metabolic functions, the algae growth medium supporting these processes, and the mass exchange system managing the flow of substances. This exchange is integral to the system's operation, as it allows for the growth of the mammalian cell culture to be partially sustained by the byproducts of the algae culture, leading to the production of a tissue product.

Step 106 involves the operation of a mass exchange system within a mammalian cell production system. This step focuses on the controlled flow of growth mediums between mammalian and algae cell cultures while maintaining separation of the cell populations.

The action described in Step 106 is the process by which the growth medium for mammalian cells is allowed to move into and out of the algae cell culture, and conversely, the algae growth medium is permitted to move into and out of the mammalian cell culture. The mass exchange system is designed with mechanisms that enable the transfer of nutrients, oxygen, carbon dioxide, and waste products between the two mediums. This system may employ semi-permeable barriers or other technologies that allow the passage of molecules necessary for cell growth and metabolic function but restrict the movement of the cells themselves.

Sub-step 106-a elaborates on the function of the mass exchange system, specifying that it operates to prevent the mixing of mammalian and algae cells. The system ensures that each cell culture receives the required inputs for sustaining growth without cross-contamination. The design of the system is based on principles such as diffusion and osmosis, which facilitate the movement of substances across a membrane or barrier.

The exchange system's operation is essential for the production of a tissue product, as it supports a symbiotic relationship between the mammalian and algae cell cultures. This relationship allows for the efficient use of waste from one culture as a resource for the other, creating a closed-loop system that mimics natural ecological cycles.

In summary, Step 106 and sub-step 106-a describe the functioning of a mass exchange system that enables the exchange of growth mediums between mammalian and algae cell cultures. This system is integral to maintaining the separate growth of mammalian cells, which are used to produce a tissue product, while leveraging the symbiotic relationship between the two cell cultures.

Step 108 involves the process of moving the mammalian and algae growth mediums through the production system. This process is carried out by mechanisms such as pumps or gravity-fed systems, which ensure that fresh nutrients reach the cells and waste products are removed. The goal of this process is to maintain homeostasis within the cell cultures, ensuring that the cells remain in a state conducive to growth, which is necessary for the production of the tissue product.

Sub-step 108-a specifies that the growth mediums enter and exit the exchange system. The exchange system is a component of the production system that allows the transfer of nutrients and waste between the mammalian and algae cultures without mixing the cell populations. The growth medium, enriched with waste containing nutrients and oxygen from the mammalian culture, enters the algae culture, providing it with components for growth. Conversely, the growth medium carrying waste rich in nutrients and carbon dioxide from the algae culture enters the mammalian culture, completing the symbiotic cycle. This exchange system is designed to prevent cross-contamination while facilitating the relationship between the mammalian and algae cell cultures.

The process of circulating and exchanging the growth mediums is manifested in the physical setup of the production system, which includes components such as pumps, conduits, and the mass exchange system. These components work together to create a closed-loop system where the growth mediums can be continuously circulated and exchanged, providing a stable environment for the production of the tissue product. The process is controlled and monitored to ensure that the flow rates, nutrient concentrations, and waste removal are within the desired ranges to optimize cell growth and tissue production.

Step 110 involves the process of generating a tissue product through the growth of mammalian cells within a cell culture system. This process is sustained by the interaction with an algae cell culture. The mammalian cells undergo cellular processes such as proliferation, differentiation, and organization to form tissue structures. These processes are supported by a growth medium that supplies necessary nutrients and maintains suitable environmental conditions.

Sub-step 110-a specifies that the growth of the mammalian cell culture, which leads to the production of the tissue product, is supported by the algae cell culture. The mammalian cell culture produces waste that includes nutrients and oxygen (Step 102-a), which are utilized by the algae cell culture. Conversely, the algae cell culture generates waste that contains nutrients and carbon dioxide (Step 104-a), which in turn supports the mammalian cell culture. This exchange of nutrients and waste is managed by a mass exchange system (Step 106-a) that allows the respective growth mediums to circulate between the mammalian and algae cell cultures without mixing the cell populations.

The process described in Step 110 and sub-step 110-a is facilitated by a system that ensures the mammalian cells receive an appropriate balance of nutrients and environmental conditions for tissue formation. The algae cell culture assists in the recycling of waste and provision of additional nutrients and carbon dioxide, which are vital for the growth of mammalian cells. The mass exchange system enables the transfer of growth mediums while maintaining the separation of mammalian and algae cells, ensuring the integrity and viability of the resulting tissue product.

In any interpretation of the claims appended hereto, it is noted that no claims or claim elements are intended to invoke or be interpreted under 35 U.S.C. 112 (f) unless the words “means for” or “step for” are explicitly used in the particular claim.

In general, any combination of disclosed features, components and methods described herein is possible. Steps of a method can be performed in any order that is physically possible.

All cited references are incorporated by reference herein. Although embodiments have been disclosed, it is not desired to be limited thereby. Rather, the scope should be determined only by the appended claims.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the present disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. The methods, kits, formulations, and devices disclosed herein can be combined in any way into systems to address the current public health emergency.

The technology described herein is further illustrated by the following examples which in no way should be construed as being further limiting. The Examples are provided to demonstrate examples of future planned work, which in some experiments is emergency work. Although methods and materials similar or equivalent to those described herein can be used in the practice or testing of this disclosure, suitable methods and materials are described below.

### Examples

The invention now being generally described with the spirit of the invention and inventive concept described and illustrated, it will be more readily understood by reference to the following examples which are included merely for purposes of illustration of certain aspects and embodiments of the present invention and are not intended to limit the invention. As can be discerned, the technology herein provides a great improvement in guiding surgical outcomes, and by helping quality of life, long-term, for patients, it is foreseen that the technology will grow.

### Example 1. Primary Bovine Satellite Cell Maintenance Protocols: Thawing PBSCs

Materials were: DMEM+10% FBS, 1% P/S, GFs (see making media, Example 11); 2×15 ml conical tube; water bath at 37° C.; cryovial of PBSCs; aspirating pipettes; serological pipettes and pipettor; and centrifuge.

The methods included the steps of:
1. 10 mL of DMEM+10% FBS, 1% P/S, and GFs were measured into a 15 ml conical tube.
2. A frozen cryovial of cells was obtained from a liquid nitrogen tank.
3. Cryovial was placed in a 37° C. water bath just until thawed, with no more frozen liquid.
4. Immediately, using a 1 mL serological pipette, the thawed cells were resuspended and quickly, gently mixed by pipetting up and down.
5. The cell mixture was added dropwise to the 10 mL of DMEM and gently shaken upon the addition of more cells.
6. Once all the thawed cell mixture was added to the DMEM, the 15 ml conical with cells in DMEM was placed in a centrifuge for 5 minutes at 10,000 RPM.
7. After centrifugation, the supernatant was aspirated. The cell pellet was resuspended in 2 mL of media.
8. A count was performed to verify cell viability.

For feeding PBSCs, the materials were: DMEM+10% FBS, 1% P/S, GFs (see making media); T-75 or T-25 flask with PBSCs; water bath at 37° C.; aspirating pipette; and serological pipettes and pipettor.

The methods further included the steps of:
9. Warm media in a 37° C. water bath for 15 minutes.
10. Remove T-flask from the incubator.
11. Working in the biosafety cabinet, aspirate media from cells.
12. For a T-25 flask a. Add 5 mL of new media
13. For a T-75 flask b. Add 11 mL of new media.

For passaging primary bovine satellite cells, the materials were: Trypsin; DMEM+10% FBS, 1% P/S, GFs (see making media); 15 ml conical tube; T-75 or T-25 flask; Water bath at 37° C.; Serological pipettes and pipettor; Aspirating pipette; and Centrifuge.

The methods (for T-75) further included the steps of:
14. Place trypsin and DMEM into a water bath for 15 minutes until warm.
15. Remove flask with cells from the incubator. View the cells under microscope to confirm confluency is about 80% and cells require passaging.
16. Remove trypsin and DMEM from the water bath and move into the biosafety cabinet.
17. Aspirate media from the flask with cells with an aspirating pipette.
18. Measure 5 mL of trypsin and place into the flask with cells.
19. Place the flask in the incubator at 37° C. for five minutes.
20. Verify that cells have detached from the flask by viewing cells under a microscope.
21. Add 5 mL of DMEM to flask to deactivate the trypsin.
22. Remove cell suspension from the flask and place it in a 15 ml conical tube.
23. The 15 ml conical with cell suspension was placed in a centrifuge for 5 minutes at 10,000 RPM. 24. After centrifugation, the supernatant was aspirated. The cell pellet was resuspended in 2 mL of media with thorough mixing to ensure full resuspension of pellet.
25. A cell count was performed using a hemocytometer. The following formula was used to calculate cell count: c. # of cells counted # of boxes counted 210,000 # of total mL resuspension=# of cells/mL.
26. Cells were plated at a density of 750,000 cells/mL.
27. 11 mL of media were added to the newly plated flask.

### Example 2. Algae Cell Culture Maintenance: Feeding C. reinhardtii Cells

Materials were as follows: TAP media; Chlamydomonas reinhardtii; Grow light (Yoyomax Inc., Model number: BW-C1YQ-8WAE); Erlenmeyer flask with metal caps; Orbital shaker platform; Serological pipettes and pipettor.

The methods further included the steps of:
28. Transfer 10 million cells to a sterile Erlenmeyer flask.
29. Add TAP to cell suspension up to 50 mL in the flask.
30. Place flask on shaking platform in a 12-h on/12-h off light cycle in a 30° C. incubator.
31. Cell suspension should be a bright, deep green. If color becomes yellow-tinted, replenish solution with 10-20 mL of TAP.

### Example 3. Hill Reaction Protocol

The materials included: DCPIP; C. reinhardtii stock culture flask; Box (to cover a 96-well plate); 6-well plate; 96-well plate; and micropipettes and tips.

The methods further included the steps of:
32. Count algae cells and determine appropriate algae culture volume needed for a 500,000 cells/mL, 5 million cells/mL, and another 5 million cells/mL concentration.
33. Take a control sample without DCPIP of each well in the 6-well-plate and placed into the first column of the 96-well-plate.
34. In a well-lit area, add 10 μL/mL, 20 μL/mL, and 50 μL/mL of DCPIP to each of the respective concentrations. There should be 4 mL of media total per well in the 6 well plate.
35. Start a timer as the DCPIP was added to the 6-well plate. Time 0 samples of 100 μL should be taken immediately after the timer is started.
36. Cover the 96-well-plate with the box to prevent further oxidation of DCPIP by algae photosynthesis.
37. Take 100 μL samples from every well on the 6-well-plate after 5, 10, 15, 20, 30, 40, and 50 minutes, covering the 96-well-plate between transferring samples and in waiting periods.
38. Read plate at 600 nm wavelength in microplate reader. Record the absorbance values for each sample at each time period to determine oxygen evolution rate.

### Example 4. Transwell Experimental Protocol

Materials were as follows: C. reinhardtii stock culture flask; cell culture flask with PBSCs; 6-well plate with 6 Transwell inserts; micropipettes and tips; parafilm; DMEM+10% FBS, 1% P/S, GFs; and an incubator at 30° C. with growth lights.

The methods further included the steps of:
39. Perform a cell count for both algae cells and PBSCs.
40. Obtain a 6-well plate with 6 Transwell inserts.
41. Plate PBSCs at 500,000 cells per well in the wells of the 6-well plate in a total volume of 3 mL.
42. Place a Transwell insert in each well of the 6-well plate.
43. Plate algae at desired concentration in the Transwell.
44. Cover Transwell with parafilm to prevent gas exchange.
45. Place the plate in a 30° C. incubator with lights on in a 12-hr on/12-hr off light cycle for four days.
46. After four days, remove Transwell inserts with algae and stain PBSCs with Hoechst according to Hoechst staining protocol (see Example 7). Take images of cells.

### Example 5. Hoechst Staining of Primary Bovine Satellite Cells

The materials included: Hoechst Stain; cell culture flask with PBSCs; PBS; micropipettes and tips; fluorescence microscope; aspirating pipettes; incubator at 37° C.; and box (to cover the flask during the staining protocol).

The methods further included the steps of:
47. Remove culture flask from the incubator.
48. Aspirate media from flask in the biosafety cabinet.
49. Wash with PBS by adding enough PBS to coat the entire surface of the flask. Aspirate.
50. Add enough PBS to coat the surface of the culture flask again.
51. Transfer 1 μL of Hoechst dye to the culture flask. Place the flask in a 37° C. incubator for 1-5 minutes, but no longer to prevent background staining.
52. Image the cells under a fluorescent microscope. d. Results: Nucleus is stained blue.

### Example 6. Live/Dead Staining of Primary Bovine Satellite Cells

Materials included: Calcein AM solution (Invitrogen) 40 μL each, 4 mM in anhydrous DMSO; ethidium homodimer-1 (Etd-1) (Invitrogen), 200 μL each, 2 mM in DMSO/H2O 1:4 (v/v); 1× Phosphate buffered saline (PBS); vortex machine; micropipettes and tips; aspirating pipettes; and box (to cover the flask during the staining protocol).

The methods further included the steps of: (adapted from Invitrogen protocol, Calcein AM-Invitrogen Procedure)
53. Let Calcein AM and EthD-1 vials sit at room temperature until they thaw.
54. Add 20 μL of EthD-1 solution to 10 mL of PBS and vortexing to give a 4 μM EthD-1 solution. Vortex.
55. 5 μL of the 4 mM calcein stock will be added to 10 mL of buffer (PBS)/ethidium solution and vortexed (approximately 2 μM calcein working solution).
56. Aspirate media from flasks.
57. Rinse flasks with PBS two times to remove excess serum.
58. Add enough staining solution to cover the bottom of each flask (2 mL for a T-25 flask). Cover the samples with a box to prevent photobleaching of the fluorophores and let incubate at room temperature for 30 minutes.
59. Once the incubation period is over, image the cells (Ex/Em 528/617 for Ethidium, 494/517 nm for Calcein). e. Results: Viable cells fluoresce green, Dead cells fluoresce red.

### Example 7. Reader Set Up

Materials included: ID reader sensors-DO and pH (Scientific Bio); 2×ID readers (from ID Developer's Kit, Scientific Bio); forceps; red calibration disk (enclosed in manufacturer's packaged with ID Developer's Kit, Scientific Bio); access to ID Data Hub Software.

The methods further included the steps of:

For plating the sensors:
60. Place the readers in the incubator and plug into adapter and computer.
61. Calibrate the reader software using the pH and DO offsets in the sensor packaging. Run the calibration on the ID Data Hub software.
62. Using forceps, place a DO sensor and a pH sensor on the bottom of the experimental flask before adding any test solutions or cells. Sensors should be placed the same distance apart as the LEDs on the ID readers.
63. Place the experimental flasks over the readers in the incubator. Align the sensors with the LEDs of the reader.
64. Set the time between points for data collection. Set the data collection mode to Scan. Set each of the patches for each reader to “Read” for DO and pH.
65. Start collecting data.

For calibration of the reader:
66. Set the software to Scan mode. Read the pH and DO constants and verify that they match the constants which came with the manufacturer's details of the sensors.
67. If the pH and DO constants do not match the constants of the sensors, Write the pH and DO sensor constants.
68. Place the reader in the environment for which the data collection will occur.
69. Click the calibration tab. Choose “Measure Offsets and Phase.”
70. Follow the instructions on the ID Data Hub interface by placing and removing the red calibration disk when prompted.
71. Repeat for each pH and DO LED on each reader.

### Example 8. Pictures of Hoechst Staining of the Transwell

FIG. 4D shows pictures of Hoechst staining of the Transwell. The images in FIG. 4D were used to calculate the cell counts for the Transwell experiments, in addition to the images shown in FIG. 3F. Values from these images are shown in FIG. 3G and FIG. 3H. Referring to FIG. 4D, images in panels A1-3 are from Transwell with no algae, images marked in Panels B1-3 were taken from Transwell with a 100:1 algae to PBSC ratio, images marked in Panels C1-3 were taken from Transwell with a 200:1 algae to PBSC ratio.

### Example 9. Sample Calculation for Determining Cell Counts from Microscope Images

Microscope fields of view were determined to be 0.2702 cm×0.3603 cm. The updated field area, accounting for 4× objective magnification was as follows: length of field of view/magnification=0.2702/4=0.06755 cm. Width of field of view/magnification=0.03603/4=0.090075 cm.

The updated total area of field of view, accounting for 8× magnification was as follows: Updated length x updated width: 0.06755 cm×0.090075 cm=0.00060846 cm². Total cells per area can be determined by multiplying the number of cells in an image x the total area of the experimental flask divided by 0.00060846.

### Example 10. Percentage do and pH Reader from Experiment 1

FIG. 4E shows the monoculture and co-culture DO versus time plot. On the Y-axis is dissolved oxygen (%), and on the X-axis is time in hours.

FIG. 4F shows the monoculture and co-culture pH versus time. On the Y-axis is pH, and on the X-axis is time in hours.

### Example 11. Pbsc Media Making Protocol

Materials included: DMEM; FBS; Penicillin/Streptomycin; Growth factors: FGF2, HGF, EGF, and IGF; 50 ml conical tubes; sterile filter and sterile flask.

The methods included the steps of:
1. In a biosafety cabinet, remove 55 mL of DMEM from the flask of DMEM and store in conical tubes. Refrigerate for later use.
2. Add 50 mL FBS to 500 mL flask of DMEM for a 10% FBS concentration.
3. Add 5 mL of Pen/Strep to the media solution for a 1% Pen/Strep concentration.
4. Obtain sterile flask and sterile filter attachment. Aseptically transfer the media with DMEM, FBS, and Pen/Strep to the flask. Attach the filter and use vacuum to transfer media through the filter.
5. Add 4 ng/ml of human fibroblast growth factor-2 (FGF2), 2.5 ng/ml recombinant human hepatocyte growth factor (HGF), 10 ng/mL recombinant human epidermal growth factor (EGF), and 5 ng/ml recombinant human insulin-like growth factor-1 (IGF).
6. Add cap and place in refrigerator until needed.

### Example 12. Calculations for Bioreactor

FIG. 4G shows the mathematical formulas used to calculate the peclet number of the fiber. Using the formulas in FIG. 4G, the Cmin/cin=0.08/0.22=0.36; A=0.8; and Pe*=0.2.

For calculating the velocity inside the lumen:

$$velocity = {Pe}^{*} \times k \times Cp \times L = 0.2 ( 1.2 ) 1.011 ( 98 ) ( 0.2 ) = 0.012 m / s = 1.2 cm / s .$$

$$K = Thermal Conductivity of DMEM ( W / mK ) , and$$

$$Cp = Heat Capacity of DMEM ( j / kgK ) .$$

For calculating the flow rate:

$$Cross - sectional Area \times Velocity {R}^{2} \times 1.2 {cm}^{2} / s = 0.012 \times 1.2 cm / s = 0.000377 {cm}^{3} / s .$$

FIG. 4H shows the mathematical equation used to calculate the shear rate. To calculate the shear rate:

$$Shear rate = 2 RoRiw / ( Ro 2 - Ri 2 ) = 2 ( 200 ) ( 100 ) ( 1.2 ) / ( 200 ) 2 - ( 100 ) 2 = 1.6 s - 1$$

For the shear force:

$$Shear Force = Shear rate \times viscosity ( DMEM ) = 1.6 s - 1 \times 0.9598 {Pa}^{*} s = 1.536 Pa .$$

### Example 13. Recycling Cell Culture Media to Decrease Bioproduction Costs

Current analyses conclude that cell culture media is the most economically critical consideration in a cultured meat production process. One such analysis suggests that, at current commercial prices, the serum free media formulation, Essential-8, would cost about $400 per liter, and about $7,535,958 per 20,000 liters. At such costs, a batch production strategy employing a 20,000 L tank would yield meat at a cost of $8,612.57 per kilogram. Novel strategies for expanding the lifespan of cell culture media may decrease the total volume required and thus decrease associated costs.

At the lab scale, cell culture media is typically replaced every two to three days as toxic metabolites such as ammonia accumulate. At operation scale, in suspension bioreactors, additional media is continuously fed into the culture to maintain a low concentration of inhibitory metabolites, such as ammonia. This approach is referred to as fed-batch culture. In pharmaceutical applications, due to quality requirements, media is typically replaced in its entirety following the completion of mammalian cell culture batches. In wastewater treatment plants, algae have been repeatedly employed to remove ammonia from wastewater. The development of continuous systems where algae can be grown in mammalian cell culture growth media at room temperature or 37° C. may extend the lifespan of the media by sequestering toxic metabolites and decrease production costs.

For cultured meat applications, cell culture media is predicted to be the biggest driver of production costs. The development of a bioprocess capable of extending the lifespan of cell culture media, that operates within or adjacent to large scale biomanufacturing operations, may decrease production costs of lab grown meat. Scientific investigation into the ability of algae to sequester nitrogen wastes, such as ammonia, from cell culture media under environmental conditions similar to those at which mammalian cells are cultured, may justify the design of continuous, symbiotic, mammalian cell bioreactors with integrated algal photobioreactors.

Satellite cells are traditionally cultured at 37° C. in DMEM F-12 containing 10% FBS and buffered at a pH of 7.4. While most photosynthetic algae are typically cultured at much lower temperatures and in more alkaline conditions, some species have adapted to more extreme environments. In the lab, the freshwater algae, Chlorella sorokiniana UTEX1230 has been cultured at an optimum range of 35° C. and at pH ranges between 6.5 and 8.5. Additionally, this highly adaptive species has been observed growing in extreme conditions in nature, suggesting that it may maintain the ability to be conditioned to grow in more saline and complex solutions such as DMEM at 37° C. FIG. 4G shows the mathematical formulas used to calculate the peclet number of the fiber. Using the formulas in FIG. 4G, the Cmin/cin=0.08/0.22=0.36; A=0.8; and Pe*=0.2.

### Experimental Methods

Algae Cultivation: An agar slant of Chlorella sorokiniana UTEX1230, isolated and deposited from “warm surface local surface water” of Austin Texas by Constantine Sorokin and Jack Meyers, was acquired from the Culture Collection of Algae at The University of Texas at Austin. All algal cultures will be maintained on an orbital shaker at 150 RPM, in a light incubator maintained at 37° C. under 12 hour day/night cycle. During the day phase, light intensity is maintained constant at between 110-130 μmol/s*m², measured by a light meter (LI-250A, LI-COR) equipped with a quantum sensor (LI-COR). The quantification of algal growth is done by counting individual algae from a high density culture from a preliminary study, serially diluted in fresh media with a hemocytometer, and collecting corresponding absorbance values at wavelength 670 using a spectrophotometer plate reader. Readings containing each media formulation with no algae are subtracted from the corresponding algae readings as blanks. A standard curve relating algal count to absorbance was created new for each growth experiment to enable rapid quantification of growth. Three absorbance readings from each culture condition will be taken and averaged at each timepoint. Initial tests were done.

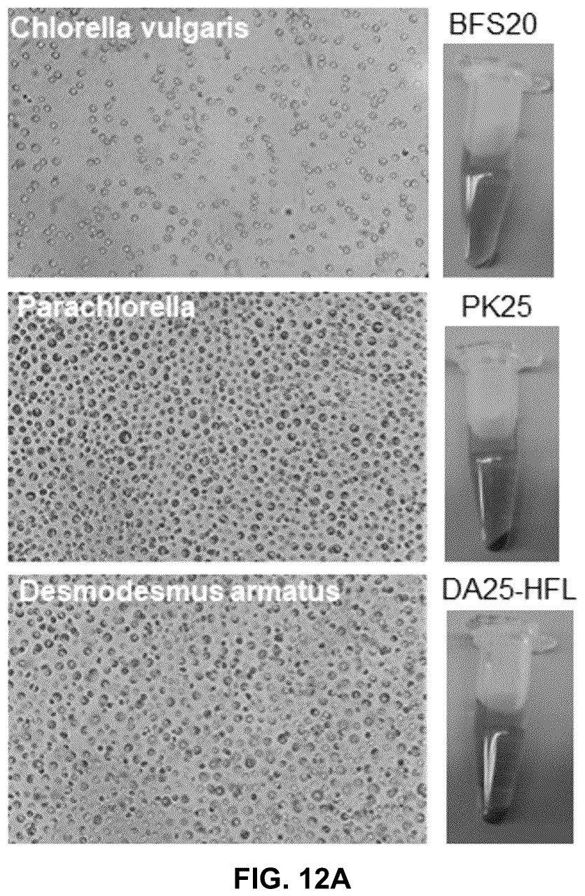

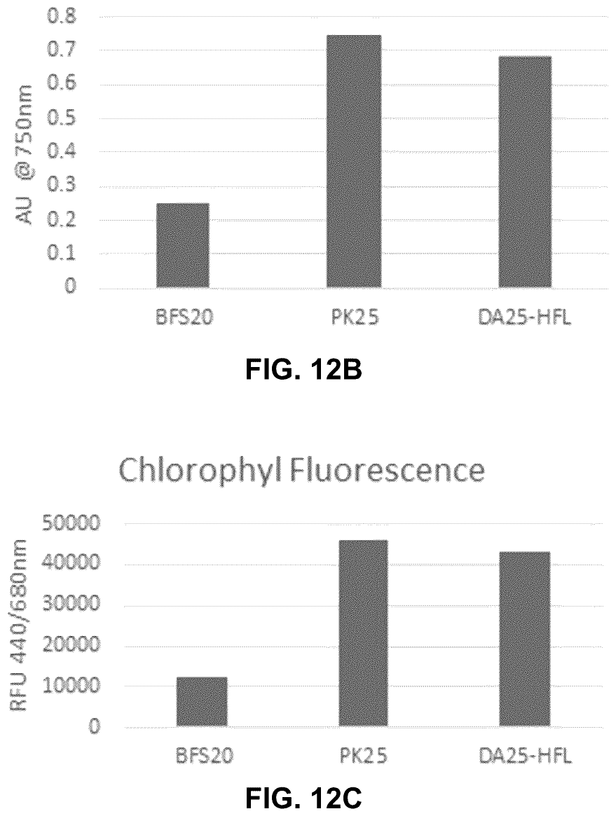

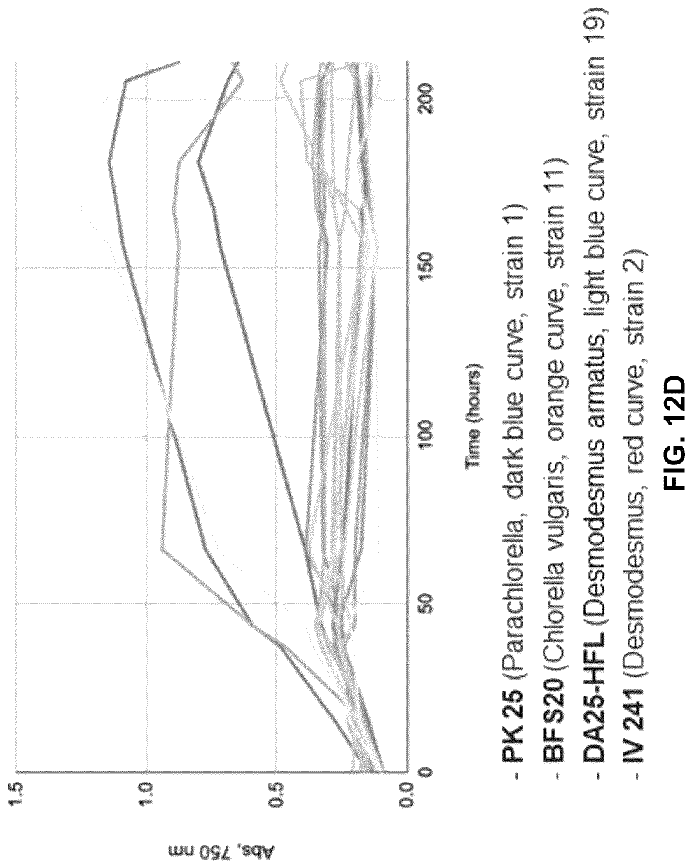

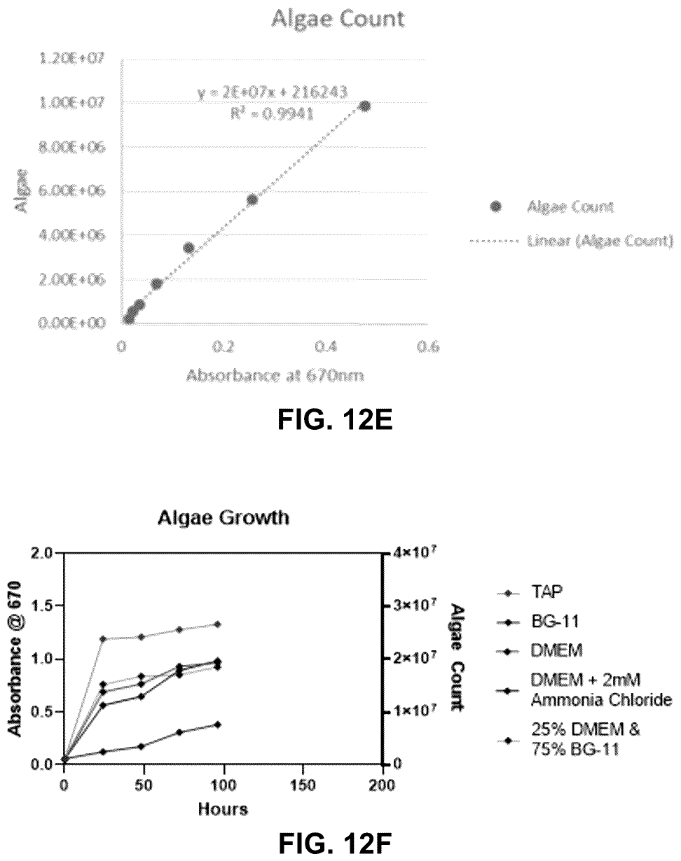

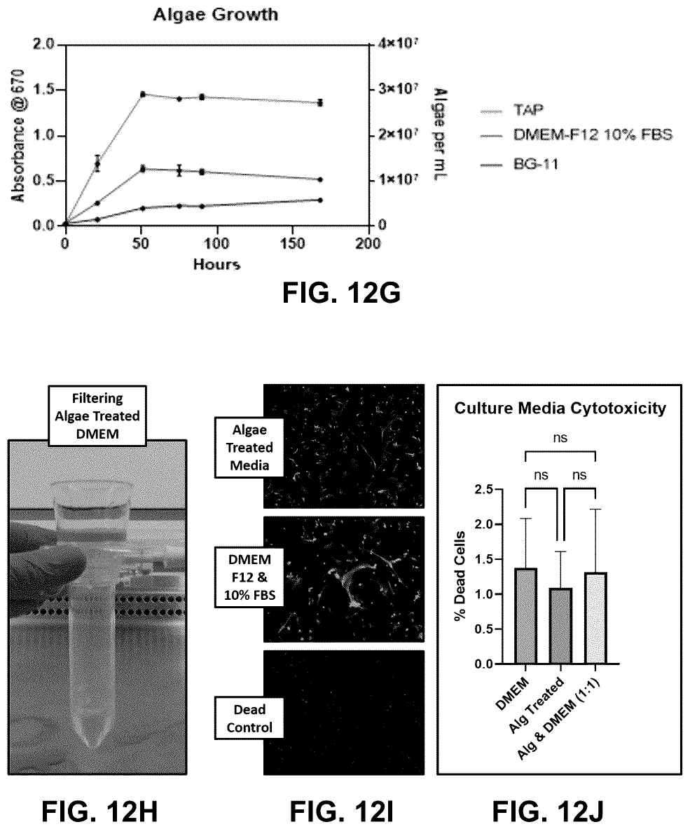

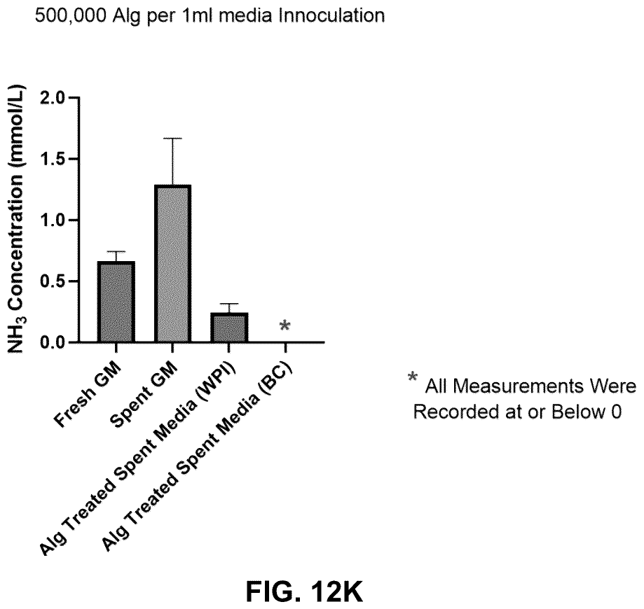

FIG. 12A shows Chlorella vulgaris (BFS20), Parachlorella (PK25), and Desmodesmus armatus (DA25-HFL) in cultures. FIG. 12B shows absorbance units at 750 nm. FIG. 12C shows a plot of chlorophyl fluorescence using 440 nm and 680 nm. FIG. 12D shows a plot of absorbance (750 nm) versus time (hours).

Using a sterile micropipette tip in a biosafety cabinet, algae will be initially inoculated into an autoclaved 125 mL Erlenmeyer flask containing 50 ml of sterile freshwater algae culture media at pH 7.4 (BG-11). The principal inoculation was maintained for 6 days before passaging into fresh BG-11 media at an inoculation density of 500,000 algae per mL. Algae will be passaged a minimum of two times post initial inoculation prior to experimental studies.

Algal Growth in Mammalian Cell Culture Media: Chlorella sorokiniana was inoculated into flasks containing phenol-red-free DMEM-F12 with 10% FBS and 1% P/S, BG-11 (pH 7.4), and TAP media (pH 7.4), and absorbance measurements were taken every 24 hours for 4 days, and then once at 7 days (n=3).

FIG. 12E shows a plot of algae count with absorbance at 670 nm on the X-axis. FIG. 12F shows a plot of absorbance at 670 nm versus hours with different media. FIG. 12G shows a plot of absorbance at 670 nm versus hours and concentration is at the right of the plot with different media. FIG. 12H shows an image of filtering algae treated DMEM. FIG. 12I shows fluorescent images of algae treated media (top), DMEM F12 and 10% FBS media (middle), and dead control (bottom). FIG. 12J shows a plot illustrating culture media cytotoxicity. FIG. 12K shows a plot of ammonia concentration with different growth media (GM) on the X-axis.

Cytotoxicity of Algal Treated Media: It is unknown if mammalian cell growth media becomes cytotoxic after Chlorella sorokiniana has been expanded within it for an extended period of time. Algae was expanded within DMEM-F12 with 10% FBS and 1% P/S for a minimum of 4 days (n=2). Algae was then removed from the algae treated media (ATM) via vacuum filtration. Primary bovine satellite cells from two biological replicates were inoculated into tissue culture treated plastic well plates at 50% confluence in fresh growth media. After 24 hours the fresh media was replaced with ATM, and control groups were given fresh growth media. Cells were maintained in an incubator at 37 C and 5% CO₂ for 72 hours. Experimental cells were stained with Hoechst and Propidium Iodide before being fixed in 4% paraformaldehyde. A positive control for dead cells was prepared by exposing additional groups to 70% ethanol for 20 minutes prior to staining. A minimum of six images were taken per group using a Cytation-1 microscope. The percentage of Hoechst-stained cell nuclei expressing positive signal for propidium iodide was calculated using ImageJ.

### REFERENCES

^{1.} IPCC. The Intergovernmental Panel on Climate Change Climate Change 2022: Mitigation of Climate Change. https://www.ipcc.ch/report/ar6/wg3/.
^{2.} Willett W., et al. Food in the Anthropocene: the EAT-Lancet Commission on healthy diets from sustainable food systems. Lancet. 2019; 393 (10170): 447-492.
^{3.} UN. UN Environment Programme Assessing the Environmental Impacts of Consumption and Production Priority Products and Materials. https://www.resourcepanel.org/reports/assessing-environmental-impacts-consumption-and-production.
^{4.} Humphreys G. Reframing climate change as a health issue. Bull World Health Organ. 2014; 92 (8): 551-552.
^{5.} Extension Texas A&M AgriLife. “The Role of Animal Agriculture on Greenhouse Gas Emissions,” Texas A&M AgriLife Extension Service, 4 Mar. 2019. https://agrilifeextension.tamu.edu/asset-external/the-role-of-animal-agriculture-on-greenhouse-gas-emissions/. Accessed 29 Apr. 2021.
^{6.} Administration U.S. Energy Information. Energy for growing and harvesting crops is a large component of farm operating costs. https://www.eia.gov/todayinenergy/detail.php?id=18431. Accessed 30 Apr. 2021.
^{7.} Post M. J. Cultured meat from stem cells: challenges and prospects. Meat Sci. 2012; 92(3): 297-301.
^{8.} Tuomisto H. L., de Mattos M. J. Environmental impacts of cultured meat production. Environ Sci Technol. 2011; 45 (14): 6117-6123.
^{9.} van der Valk J., et al. Optimization of chemically defined cell culture media-replacing fetal bovine serum in mammalian in vitro methods. Toxicol In Vitro. 2010; 24 (4): 1053-1063.
^{10.} Richelle A., Lewis N. E. Improvements in protein production in mammalian cells from targeted metabolic engineering. Curr Opin Syst Biol. 2017; 6:1-6.
^{11.} Haraguchi Y., et al. Thicker three-dimensional tissue from a “symbiotic recycling system” combining mammalian cells and algae. Sci Rep. 2017; 7:41594.
^{12.} Okamoto Y., et al. Mammalian cell cultivation using nutrients extracted from microalgae. Biotechnol Prog. 2020; 36 (2): e2941.
^{13.} Nierenberg Danielle. Factory farming in the developing world. World Watch. 2003; 16 (3): 10-19.
^{14.} Anomaly Jonathan. What's Wrong With Factory Farming? Public Health Ethics. 2014; 8 (3): 246-254.
^{15.} D'Silva Joyce. Chapter: Modern Farming Practices and Animal Welfare in Book: Ethics, Law and Society. 2017, (9781315094328).
^{16.} Our World in Data Hannah Ritchie. How much of the world's land would we need in order to feed the global population with the average diet of a given country? https://ourworldindata.org/agricultural-land-by-global-diets. Accessed 20 Apr. 2021.
^{17.} Wallace J. S. Increasing agricultural water use efficiency to meet future food production. Agriculture, Ecosystems & Environment. 2000; 82 (1): 105-119.
^{18.} EPA. How We Use Water. https://www.epa.gov/watersense/how-we-use-water. Accessed 30 Apr. 2021.
^{19.} Allan Scott J., et al. Bioprocess Design Considerations for Cultured Meat Production With a Focus on the Expansion Bioreactor. Frontiers in Sustainable Food Systems. 2019; 3.
^{20.} Jones Kate E., et al. Global trends in emerging infectious diseases. Nature. 2008; 451 (7181): 990-993.
^{21.} Zuzworsky Rose. From the marketplace to the dinner plate: The economy, theology, and factory farming. Journal of Business Ethics. 2001; 29 (1-2): 177-188.
^{22.} Post M. J. Cultured beef: medical technology to produce food. J Sci Food Agric. 2014; 94 (6): 1039-1041.
^{23.} Ben-Arye Tom, Levenberg Shulamit. Tissue Engineering for Clean Meat Production. Frontiers in Sustainable Food Systems. 2019; 3.
^{24.} Stephens N., et al. Bringing cultured meat to market: Technical, socio-political, and regulatory challenges in cellular agriculture. Trends Food Sci Technol. 2018; 78:155-166.
^{25.} Paarsch Harry J. Micro-economic Models of Beef Supply. Canadian Journal of Economics. 1985; 18 Number 3:636-651.
^{26.} Ibarburu-Blanc M. A., Lawrence, J. D., Busby, W. D. & Strohbehn, D. R. Assessing the Cost of Beef Quality Revisited. Iowa State University Animal Industry Report. 2010; 7 (1).
^{27.} Van Loo Ellen J., et al. Consumer preferences for farm-raised meat, lab-grown meat, and plant-based meat alternatives: Does information or brand matter? Food Policy. 2020; 95:101931.
^{28.} Hughes David C., et al. Testosterone enables growth and hypertrophy in fusion impaired myoblasts that display myotube atrophy: deciphering the role of androgen and IGF-I receptors Biogerontology. 2016; 17 (3): 619-639.
^{29.} Collins Charlotte A., et al. A Population of Myogenic Stem Cells That Survives Skeletal Muscle Aging. STEM CELLS. 2007; 25 (4): 885-894.
^{30.} Post Kristel J. M. Boonen and Mark J. The Muscle Stem Cell Niche: Regulation of Satellite Cells During Regeneration. Tissue Engineering Part B: Reviews. 2008; 14 (4): 419-431.
^{31.} Listrat A., et al. How Muscle Structure and Composition Influence Meat and Flesh Quality. ScientificWorldJournal. 2016; 2016:3182746.
^{32.} Claeys E., et al. Quantification of fresh meat peptides by SDS-PAGE in relation to ageing time and taste intensity. Meat Science. 2004; 67 (2): 281-288.
^{33.} S. Mottram Donald. Flavour formation in meat and meat products: a review. Food Chemistry. 1998; 62 (4): 415-424.
^{34.} Ma Peter X. Scaffolds for tissue fabrication. Materials Today. 2004; 7 (5): 30-40.
^{35.} Gershlak Joshua R., et al. Crossing kingdoms: Using decellularized plants as perfusable tissue engineering scaffolds. Biomaterials. 2017; 125:13-22.
^{36.} P. D. Edelman D. C. McFarland, V. A. Mironov, and J. G. Matheny. Commentary: In Vitro-Cultured Meat Production. Tissue Engineering. 2005; 11 (5-6): 659-662.
^{37.} Post M. J., Hocquette J. F. Chapter 16-New Sources of Animal Proteins: Cultured Meat. In: Purslow Peter P., ed. New Aspects of Meat Quality. Woodhead Publishing; 2017:425-441.
^{38.} Kolkmann A. M., et al. Serum-free media for the growth of primary bovine myoblasts. Cytotechnology. 2020; 72 (1): 111-120.
^{39.} Chriki S., Hocquette J. F. The Myth of Cultured Meat: A Review. Front Nutr. 2020; 7:7.
^{40.} Mantovani Katia Bilodeau and Diego. Bioreactors for Tissue Engineering: Focus on Mechanical Constraints. A Comparative Review. Tissue Engineering. 2006; 12 (8): 2367-2383.
^{41.} Blaby Ian, et al. Modes of Culture/Microbial. In: 2011:303-314.
^{42.} Paolo G. Antolli Zhiming Liu, M. R. Spier. “Application of Different Types of Bioreactors in Bioprocesses”; Bioreactors: design, properties, and applications. 2012, (9781621002277).
^{43.} Salgado A. J., et al. Bone tissue engineering: state of the art and future trends. Macromol Biosci. 2004; 4 (8): 743-765.
^{44.} Spect L. An analysis of culture medium costs and production volumes for cultivated meat. The Good Food Institute. https://www.gfi.org/resources.php?audience=scientists. Accessed 12 Oct. 2020.
^{45.} Levenberg Shulamit, et al. Engineering vascularized skeletal muscle tissue. Nature Biotechnology. 2005; 23 (7): 879-884.
^{46.} Zhanqiu Yang, Hai-Rong Xiong. Culture Conditions and Types of Growth Media for Mammalian Cells. In: Luca Ceccherini-Nelli, Barbara Matteoli, eds. Biomedical Tissue Culture. Rijeka: IntechOpen; 2012: Ch. 1.
^{47.} Verma Anju, et al. Chapter 14-Animal tissue culture principles and applications. In: Verma Ashish S., Singh Anchal, eds. Animal Biotechnology (Second Edition). Boston: Academic Press; 2020:269-293.
^{48.} Yao T., Asayama Y. Animal-cell culture media: History, characteristics, and current issues. Reprod Med Biol. 2017; 16 (2): 99-117.
^{49.} Yablonka-Reuveni Z., et al. Fibroblast growth factor promotes recruitment of skeletal muscle satellite cells in young and old rats. J Histochem Cytochem. 1999; 47 (1): 23-42.
^{50.} Montarras Didier, et al. Lying low but ready for action: the quiescent muscle satellite cell. The FEBS Journal. 2013; 280 (17): 4036-4050.
^{51.} Pawlikowski Bradley, et al. Regulation of skeletal muscle stem cells by fibroblast growth factors. Developmental Dynamics. 2017; 246 (5): 359-367.
^{52.} Danoviz M. E., Yablonka-Reuveni Z. Skeletal muscle satellite cells: background and methods for isolation and analysis in a primary culture system. Methods Mol Biol. 2012; 798:21-52.
^{53.} Francis Geoffrey L. Albumin and mammalian cell culture: implications for biotechnology applications. Cytotechnology. 2010; 62 (1): 1-16.
^{54.} Guedes Sofia, et al. Oxidation of bovine serum albumin: Identification of oxidation products and structural modifications. Rapid communications in mass spectrometry: RCM. 2009; 23:2307-2315.
^{55.} Al-Rubeai M., et al. Death mechanisms of animal cells in conditions of intensive agitation. Biotechnol Bioeng. 1995; 45 (6): 463-472.
^{56.} Taverna M., et al. Specific antioxidant properties of human serum albumin. Ann Intensive Care. 2013; 3 (1): 4.
^{57.} Fernandez-de-Cossio-Diaz Jorge, et al. Characterizing steady states of genome-scale metabolic networks in continuous cell cultures. PLOS Computational Biology. 2017; 13 (11): e1005835.
^{58.} Freund N. W., Croughan M. S. A Simple Method to Reduce both Lactic Acid and Ammonium Production in Industrial Animal Cell Culture. Int J Mol Sci. 2018; 19 (2).
^{59.} Goers Lisa, et al. Co-culture systems and technologies: taking synthetic biology to the next level. Journal of The Royal Society Interface. 2014; 11 (96): 20140065.
^{60.} Wu Min-Hsien, et al. Microfluidic cell culture systems for drug research. Lab on a Chip. 2010; 10 (8): 939-956.
^{61.} Bogdanowicz D. R., Lu H. H. Studying cell-cell communication in co-culture. Biotechnol J. 2013; 8 (4): 395-396. 62. Miki Y., et al. The advantages of co-culture over mono cell culture in simulating in vivo environment. J Steroid Biochem Mol Biol. 2012; 131 (3-5): 68-75.
^{63.} Jones J. Andrew, Wang Xin. Use of bacterial co-cultures for the efficient production of chemicals. Current Opinion in Biotechnology. 2018; 53:33-38.
^{64.} Vinale F., et al. Co-Culture of Plant Beneficial Microbes as Source of Bioactive Metabolites. Scientific Reports. 2017; 7 (1): 14330.
^{65.} Sigma-Aldrich. Dulbecco's Modified Eagle's Medium (DME) Formulation. Sigma Aldrich. https://www.sigmaaldrich.com/life-science/cell-culture/learning-center/media-formulations/dme.html. Accessed 15 Oct. 2020.
^{66.} Scott Christopher. Misconceptions about Aerobic and Anaerobic Energy Expenditure. Journal of the International Society of Sports Nutrition. 2005; 2 (2): 32.
^{67.} Olah George A., et al. Anthropogenic Chemical Carbon Cycle for a Sustainable Future. Journal of the American Chemical Society. 2011; 133 (33): 12881-12898.
^{68.} Fowler David, et al. The global nitrogen cycle in the twenty-first century. Philosophical Transactions of the Royal Society B: Biological Sciences. 2013; 368 (1621): 20130164.
^{69.} Bergner H. [Nitrogen metabolism and its control mechanisms]. Arch Tierernahr. 1989; 39 (4-5): 377-392.
^{70.} Beatty P. H., et al. Understanding Plant Nitrogen Metabolism through Metabolomics and Computational Approaches. Plants (Basel). 2016; 5 (4).
^{71.} Mazard Sophie, et al. Tiny Microbes with a Big Impact: The Role of Cyanobacteria and Their Metabolites in Shaping Our Future. Marine Drugs. 2016; 14 (5): 97.
^{72.} Delgadillo-Mirquez Liliana, et al. Nitrogen and phosphate removal from wastewater with a mixed microalgae and bacteria culture. Biotechnology Reports. 2016; 11:18-26.
^{73.} Rodolfi L., et al. Growth medium recycling in Nannochloropsis sp. mass cultivation. Biomol Eng. 2003; 20 (4-6): 243-248.
^{74.} Rajendrudu G., et al. Isolation of intact mesophyll cells from the leaves of higher plants. Proceedings of the Indian Academy of Sciences-Section A Part 3 Mathematical sciences. 1979; 88:143-154.
^{75.} Mustafa Natali, et al. Initiation, growth and cryopreservation of plant cell suspension cultures. Nature protocols. 2011; 6:715-742.
^{76.} Leonelli S. Arabidopsis, the botanical Drosophila: from mouse cress to model organism. Endeavour. 2007; 31 (1): 34-38.
^{77.} Barkla B. J., et al. Growing Arabidopsis in vitro: cell suspensions, in vitro culture, and regeneration. Methods Mol Biol. 2014; 1062:53-62.
^{78.} Negrutiu I., Jacobs M. Arabidopsis thaliana as a model system in somatic cell genetics II. Cell suspension culture. Plant Science Letters. 1977; 8 (1): 7-15.
^{79.} Baird Marianne F, et al. Creatine-kinase- and exercise-related muscle damage implications for muscle performance and recovery. Journal of nutrition and metabolism. 2012; 2012.
^{80.} Peltier Gilles, Thibault Pierre. Ammonia Exchange and Photorespiration in Chlamydomonas. Plant Physiology. 1983; 71 (4): 888-892.
^{81.} Fernandez Emilio, Galvan Aurora. Nitrate Assimilation in <i>Chlamydomonas</i>. Eukaryotic Cell. 2008; 7 (4): 555-559.
^{82.} Bellido-Pedraza Carmen M., et al. Chlamydomonas reinhardtii, an Algal Model in the Nitrogen Cycle. Plants. 2020; 9 (7): 903.
^{83.} Hopfner Ursula, et al. Development of photosynthetic biomaterials for in vitro tissue engineering. Acta Biomaterialia. 2014; 10 (6): 2712-2717.
^{84.} Tam N. F. Y., Wong Y. S. Effect of ammonia concentrations on growth of Chlorella vulgaris and nitrogen removal from media. Bioresource Technology. 1996; 57 (1): 45-50.
^{85.} Perez-Garcia Octavio, et al. Efficiency of Growth and Nutrient Uptake From Wastewater by Heterotrophic, Autotrophic, and Mixotrophic Cultivation of Chlorella Vulgaris Immobilized With Azospirillum Brasilense1. Journal of Phycology. 2010; 46 (4): 800-812.
^{86.} Darwish Randa, et al. Chlamydomonas reinhardtii Is a Potential Food Supplement with the Capacity to Outperform Chlorella and Spirulina. Applied Sciences. 2020; 10 (19): 6736.
^{87.} Ji Xing, et al. Competition between cyanobacteria and green algae at low versus elevated CO₂: who will win, and why? Journal of Experimental Botany. 2017; 68 (14): 3815-3828.
^{88.} Bláha L., et al. Toxins produced in cyanobacterial water blooms-toxicity and risks. Interdiscip Toxicol. 2009; 2 (2): 36-41.
^{89.} Bui T., et al. Warming Affects Growth Rates and Microcystin Production in Tropical Bloom-Forming Microcystis Strains. Toxins (Basel). 2018; 10 (3).
^{90.} Baeshen N. A., et al. Cell factories for insulin production. Microb Cell Fact. 2014; 13:141.
^{91.} Wegmann Udo, et al. Use of genetically modified bacteria for drug delivery in humans: Revisiting the safety aspect. Scientific Reports. 2017; 7 (1): 2294.
^{92.} Xu Jay, et al. Platforms for Plant-Based Protein Production. In: 2016:1-40.
^{93.} ISO. ISO 22000-Food safety management. https://www.iso.org/iso % 22000-food-safety-management.html. Accessed 29 Apr. 2021.
^{94.} ISO. ISO 11737-2:2019. https://www.iso.org/standard/70801.html. Accessed 29 Apr. 2021.
^{95.} ISO. ISO 31000-Risk management. https://www.iso.org/iso-31000-risk-management.html. Accessed 29 Apr. 2021.
^{96.} FDA. CFR-Code of Federal Regulations Title 21. https://www.accessdata.fda.gov/scripts/cdrh/cfdocs/cfcfr/CFRSearch.cfm?fr=210.1. Accessed 29 Apr. 2021.
^{97.} Service Food Safety and Inspection. Food Safety and Inspection Service. https://www.fsis.usda.gov/. Accessed 29 Apr. 2021. 98. Institute Legal Information. 21 U.S. Code § 350 g-Hazard analysis and risk-based preventive controls. https://www.law.cornell.edu/uscode/text/21/350g. Accessed 29 Apr. 2021.
^{99.} Bellido-Pedraza Carmen M., et al. Chlamydomonas reinhardtii, an Algal Model in the Nitrogen Cycle. Plants. 2020; 9 (7): 903.
^{100.} Peltier Gilles, Thibault Pierre. Ammonia Exchange and Photorespiration in Chlamydomonas. Plant Physiology. 1983; 71 (4): 888-892.
^{101.} Fernandez Emilio, Galvan Aurora. Nitrate Assimilation in Chlamydomonas. Eukaryotic Cell. 2008; 7 (4): 555-559.
^{102.} Vítová M., et al. Chlamydomonas reinhardtii: duration of its cell cycle and phases at growth rates affected by temperature. Planta. 2011; 234 (3): 599-608.
^{103.} Labs PhytoTech. TAP (Tris-Acetate-Phosphate) Medium. https://phytotechlab.com/tap-tris-acetate%phosphate-medium.html. Accessed 16 Apr. 2021.
^{104.} Kang Seongsu, et al. A novel electroporation system for efficient molecular delivery into Chlamydomonas reinhardtii with a 3-dimensional microelectrode. Scientific Reports. 2015; 5 (1): 15835.
^{105.} Ferenczi Aron, et al. Efficient targeted DNA editing and replacement in <i>Chlamydomonas reinhardtii</i>using Cpf1 ribonucleoproteins and single-stranded DNA. Proceedings of the National Academy of Sciences. 2017; 114 (51): 13567-13572.
^{106.} Dehghani Jaber, et al. Stable transformation of Spirulina (Arthrospira) platensis: a promising microalga for production of edible vaccines. Applied microbiology and biotechnology. 2018; 102:9267-9278.
^{107.} Boussiba Sammy, Gibson Jane. Ammonia translocation in cyanobacteria. FEMS Microbiology Letters. 1991; 88 (1): 1-14.
^{108.} Sanchez-Luna Luis Dante, et al. Influence of pH, temperature, and urea molar flowrate on Arthrospira platensis fed-batch cultivation: a kinetic and thermodynamic approach. Biotechnology and Bioengineering. 2007; 96 (4): 702-711.
^{109.} Jeamton Wattana, et al. Overcoming Intrinsic Restriction Enzyme Barriers Enhances Transformation Efficiency in Arthrospira platensis C1. Plant and Cell Physiology. 2017; 58 (4): 822-830.
^{110.} Rawat Suman R., et al. AtAMT1 gene expression and NH4+ uptake in roots of Arabidopsis thaliana: evidence for regulation by root glutamine levels. The Plant Journal. 1999; 19 (2): 143-152.
^{111.} Sello Simone, et al. A Rapid and Efficient Method to Obtain Photosynthetic Cell Suspension Cultures of Arabidopsis thaliana. Frontiers in Plant Science. 2017; 8.
^{112.} Sangwan Rajbir S., et al. Characterization of competent cells and early events of Agrobacterium-mediated genetic transformation in Arabidopsis thaliana. Planta. 1992; 188 (3): 439-456.
^{113.} Schirawski J., et al. An improved protocol for the preparation of protoplasts from an established Arabidopsis thaliana cell suspension culture and infection with RNA of turnip yellow mosaic tymovirus: a simple and reliable method. J Virol Methods. 2000; 86 (1): 85-94.
^{114.} Fagang Jiang, et al. Effects of visible light on cultured bovine trabecular cells. Current Medical Science. 2004; 24 (2): 178-180.
^{115.} Han Ping, Bartels David M. Temperature dependence of oxygen diffusion in H 2 O and D 2 O. The Journal of physical chemistry. 1996; 100 (13): 5597-5602.
^{116.} Shipley R. J., et al. A strategy to determine operating parameters in tissue engineering hollow fiber bioreactors. Biotechnol Bioeng. 2011; 108 (6): 1450-1461.
^{117.} Ahn J., et al. Effects of plant extracts on microbial growth, color change, and lipid oxidation in cooked beef. Food Microbiol. 2007; 24 (1): 7-14.
^{118.} Schulze P. S. C., et al. Flashing LEDs for Microalgal Production. Trends Biotechnol. 2017; 35 (11): 1088-1101.
^{119.} Sigma-Aldrich. Parafilm-M. P7793. https://www.sigmaaldrich.com/content/dam/sigma-aldrich/docs/Sigma/Product_Information_Sheet/1/p7543pis.pdf. Accessed 1 May 2021.
^{120.} Liu J. K. The history of monoclonal antibody development-Progress, remaining challenges and future innovations. Ann Med Surg (Lond). 2014; 3 (4): 113-116.
^{121.} Zhu Marie M., et al. Industrial Production of Therapeutic Proteins: Cell Lines, Cell Culture, and Purification. In: Handbook of Industrial Chemistry and Biotechnology. 2017:1639-1669.
^{122.} Han F., et al. Tissue Engineering and Regenerative Medicine: Achievements, Future, and Sustainability in Asia. Front Bioeng Biotechnol. 2020; 8:83.
^{123.} Treich N. Cultured Meat: Promises and Challenges. Environ Resour Econ (Dordr). 2021; 79 (1): 33-61.
^{124.} Nobre Farley Simon. Cultured meat and the sustainable development goals. Trends in Food Science & Technology. 2022; 124:140-153.
^{125.} Potnis K. C., et al. Cost-effectiveness of chimeric antigen receptor T-cell therapy in adults with relapsed or refractory follicular lymphoma. Blood Adv. 2023; 7 (5): 801-810.
^{126.} Failla Morgan, et al. Evaluation of public submissions to the USDA for labeling of cell-cultured meat in the United States. Frontiers in Nutrition. 2023; 10.
^{127.} Hong T. K., et al. Current Issues and Technical Advances in Cultured Meat Production: A Review. Food Sci Anim Resour. 2021; 41 (3): 355-372.
^{128.} Yao Tatsuma, Asayama Yuta. Animal-cell culture media: History, characteristics, and current issues. Reprod Med Biol. 2017; 16 (2): 99-117. doi: 10.1002/rmb2.12024.
^{129.} Schneider M., et al. The importance of ammonia in mammalian cell culture. J Biotechnol. 1996; 46 (3): 161-185.
^{130.} Xu W. J., et al. Progress in fed-batch culture for recombinant protein production in CHO cells. Appl Microbiol Biotechnol. 2023; 107 (4): 1063-1075.
^{131.} Domján Júlia, et al. Real-time amino acid and glucose monitoring system for the automatic control of nutrient feeding in CHO cell culture using Raman spectroscopy. Biotechnology Journal. 2022; 17 (5): 2100395.
^{132.} Specht Liz. Liz Specht, Ph.D. An analysis of culture medium costs and production volumes for cultivated meat. The Good Food Institute. https://gfi.org/wp-content/uploads/2021/01/clean-meat-production-volume-and-medium-cost.pdf.
^{133.} Stout Andrew J., et al. Simple and effective serum-free medium for sustained expansion of bovine satellite cells for cell cultured meat. Communications Biology. 2022; 5 (1): 466.
^{134.} Abdelfattah A., et al. Microalgae-based wastewater treatment: Mechanisms, challenges, recent advances, and future prospects. Environ Sci Ecotechnol. 2023; 13:100205.
^{135.} Ganesan R., et al. A review on prospective production of biofuel from microalgae. Biotechnol Rep (Amst). 2020; 27: e00509.
^{136.} Li X., et al. Effect of ammonium nitrogen on microalgal growth, biochemical composition and photosynthetic performance in mixotrophic cultivation. Bioresour Technol. 2019; 273:368-376.
^{137.} Haraguchi Yuji, et al. A circular cell culture system using microalgae and mammalian myoblasts for the production of sustainable cultured meat. Arch Microbiol. 2022; 204 (10): 615. doi: 10.1007/s00203-022-03234-9.
^{138.} Au-Motohashi Norio, et al. Isolation, Culture, and Transplantation of Muscle Satellite Cells. JoVE. 2014 (86): e50846.
^{139.} Ota M., et al. Effects of light intensity and temperature on photoautotrophic growth of a green microalga, Chlorococcum littorale. Biotechnol Rep (Amst). 2015; 7:24-29.
^{140.} Ziganshina E. E., et al. Growth Characteristics of Chlorella sorokiniana in a Photobioreactor during the Utilization of Different Forms of Nitrogen at Various Temperatures. Plants (Basel). 2022; 11 (8).
^{141.} Bulynina Svetlana S., et al. Growth Efficiency of Chlorella sorokiniana in Synthetic Media and Unsterilized Domestic Wastewater. Bio Tech. 2023; 12 (3): 53.
^{142.} Ziganshina E. E., et al. Assessment of Chlorella sorokiniana Growth in Anaerobic Digester Effluent. Plants (Basel). 2021; 10 (3).
^{143.} Qiu Renhe, et al. Effects of pH on cell growth, lipid production and CO₂ addition of microalgae Chlorella sorokiniana. Algal Research. 2017; 28: Medium: X; Size: p. 192-199.
^{144.} Park J. E., et al. The Contribution Ratio of Autotrophic and Heterotrophic Metabolism during a Mixotrophic Culture of Chlorella sorokiniana. Int J Environ Res Public Health. 2021; 18 (3).
^{145.} Gershlak J. R., et al. Crossing kingdoms: Using decellularized plants as perfusable tissue engineering scaffolds. Biomaterials. 2017; 125:13-22.
^{146.} Perreault Luke R., et al. Repurposing agricultural waste as low-cost cultured meat scaffolds. Frontiers in Food Science and Technology. 2023; 3.
^{147.} Thyden Richard, et al. An Edible, Decellularized Plant Derived Cell Carrier for Lab Grown Meat. Applied Sciences. 2022; 12 (10): 5155.
^{148.} Rathore Anurag S., et al. Bioprocess Control: Current Progress and Future Perspectives. Life. 2021; 11 (6): 557.
^{149.} Lahue C., et al. History and Domestication of Saccharomyces cerevisiae in Bread Baking. Front Genet. 2020; 11:584718.
^{150.} Ho C. W., et al. Varieties, production, composition and health benefits of vinegars: A review. Food Chem. 2017; 221:1621-1630.
^{151.} Savaiano D. A., Hutkins R. W. Yogurt, cultured fermented milk, and health: a systematic review. Nutr Rev. 2021; 79 (5): 599-614.
^{152.} Additives EFSA Panel on Food, et al. Re-evaluation of xanthan gum (E 415) as a food additive. EFSA Journal. 2017; 15 (7): e04909.
^{153.} Abedi E., Hashemi S. M. B. Lactic acid production-producing microorganisms and substrates sources-state of art. Heliyon. 2020; 6 (10): e04974.
^{154.} Humbird D. Scale-up economics for cultured meat. Biotechnol Bioeng. 2021; 118 (8): 3239-3250.
^{155.} Slivac I., et al. Influence of different ammonium, lactate and glutamine concentrations on CCO cell growth. Cytotechnology. 2010; 62 (6): 585-594.
^{156.} Capiaumont J., et al. Methods for reducing the ammonia in hybridoma cell cultures. J Biotechnol. 1995; 39 (1): 49-58.
^{157.} DeBerardinis R. J., Cheng T. Q's next: the diverse functions of glutamine in metabolism, cell biology and cancer. Oncogene. 2010; 29 (3): 313-324.
^{158.} Chen P., Harcum S. W. Effects of elevated ammonium on glycosylation gene expression in CHO cells. Metab Eng. 2006; 8 (2): 123-132.
^{159.} Imamoto Y., et al. Advantages of AlaGln as an additive to cell culture medium: use with anti-CD20 chimeric antibody-producing POTELLIGENT™ CHO cell lines. Cytotechnology. 2013; 65 (1): 135-143.
^{160.} Oslan S. N. H., et al. Integrated Stirred-Tank Bioreactor with Internal Adsorption for the Removal of Ammonium to Enhance the Cultivation Performance of gdhA Derivative Pasteurella multocida B: 2. Microorganisms. 2020; 8 (11).
^{161.} Osborne M. G., et al. Removal of Toxic Volatile Compounds in Batch Culture Prolongs Stationary Phase and Delays Death of Escherichia coli. Appl Environ Microbiol. 2021; 87 (24): e0186021.
^{162.} Yamaguchi Masakuni, et al. Proton-based solid acids for ammonia absorption in ammonia water. International Journal of Hydrogen Energy. 2020; 45 (41): 22189-22194.
^{163.} Nayve F. R., Jr., et al. Selective removal of ammonia from animal cell culture broth. 163. Cytotechnology. 1991; 6 (2): 121-130.
^{164.} Brose D. J., van Eikeren P. A membrane-based method for removal of toxic ammonia from mammalian-cell culture. Appl Biochem Biotechnol. 1990; 24-25:457-468.
^{165.} Soto-Herranz María, et al. Comparison of the Ammonia Trapping Performance of Different Gas-Permeable Tubular Membrane System Configurations. Membranes. 2022; 12 (11): 1104.
^{166.} Chang Y. H., et al. In-situ removal of ammonium and lactate through electrical means for hybridoma cultures. Biotechnol Bioeng. 1995; 47 (3): 308-318.
^{167.} Myers G. M., et al. Nutrient recovery in cultured meat systems: Impacts on cost and sustainability metrics. Front Nutr. 2023; 10:1151801.
^{168.} Haraguchi Y., Shimizu T. Microalgal culture in animal cell waste medium for sustainable ‘cultured food’ production. Arch Microbiol. 2021; 203 (9): 5525-5532.
^{169.} Sandberg T. E., et al. The emergence of adaptive laboratory evolution as an efficient tool for biological discovery and industrial biotechnology. Metab Eng. 2019; 56:1-16.
^{170.} Hopes Amanda, Mock Thomas. Evolution of Microalgae and Their Adaptations in Different Marine Ecosystems. In: Encyclopedia of Life Sciences. 1-9.
^{171.} Metsoviti M. N., et al. Effect of Light Intensity and Quality on Growth Rate and Composition of Chlorella vulgaris. Plants (Basel). 2019; 9 (1).
^{172.} Holdmann Claudia, et al. Keeping the light energy constant-Cultivation of Chlorella sorokiniana at different specific light availabilities and different photoperiods. Algal Research. 2018; 29:61-70.
^{173.} Nawrocki Wojciech J., et al. Molecular origins of induction and loss of photoinhibition-related energy dissipation q<sub></sub>. Science Advances. 2021; 7 (52): eabj0055.
^{174.} Lacroux Julien, et al. Mixotrophic Growth of Chlorella sorokiniana on Acetate and Butyrate: Interplay Between Substrate, C: N Ratio and pH. Frontiers in Microbiology. 2021; 12.
^{175.} Cecchin M., et al. Molecular basis of autotrophic vs mixotrophic growth in Chlorella sorokiniana. Scientific Reports. 2018; 8 (1): 6465.
^{176.} Piasecka A., Baier A. Metabolic and Proteomic Analysis of Chlorella sorokiniana, Chloroidium saccharofilum, and Chlorella vulgaris Cells Cultured in Autotrophic, Photoheterotrophic, and Mixotrophic Cultivation Modes. Molecules. 2022; 27 (15).
^{177.} Straka L., Rittmann B. E. Effect of culture density on biomass production and light utilization efficiency of Synechocystis sp. PCC 6803. Biotechnol Bioeng. 2018; 115 (2): 507-511.
^{178.} Saccardo A., et al. Microalgae growth in ultra-thin steady-state continuous photobioreactors: assessing self-shading effects. Front Bioeng Biotechnol. 2022; 10:977429.
^{179.} Chisti Y. Constraints to commercialization of algal fuels. J Biotechnol. 2013; 167 (3): 201-214.
^{180.} Chisti Y. Biodiesel from microalgae. Biotechnol Adv. 2007; 25 (3): 294-306.
^{181.} Salvador W. O. S., et al. Bioprocess Economic Modeling: Decision Support Tools for the Development of Stem Cell Therapy Products. Bioengineering (Basel). 2022; 9 (12).
^{182.} Mitra S., Murthy G. S. Bioreactor control systems in the biopharmaceutical industry: a critical perspective. Syst Microbiol Biomanuf. 2022; 2 (1): 91-112.
^{183.} Pereira S., et al. Impact of CHO Metabolism on Cell Growth and Protein Production: An Overview of Toxic and Inhibiting Metabolites and Nutrients. Biotechnol J. 2018; 13 (3): e1700499.
¹⁸⁴. Schnellbaecher A., et al. Vitamins in cell culture media: Stability and stabilization strategies. Biotechnol Bioeng. 2019; 116 (6): 1537-1555.
¹⁸⁵. Ghasemi N., et al. Optimization of Key Factors in Serum Free Medium for Production of Human Recombinant GM-CSF Using Response Surface Methodology. Iran J Pharm Res. 2019; 18 (Suppl1): 146-156.
^{186.} Baltz J. M. Media composition: salts and osmolality. Methods Mol Biol. 2012; 912:61-80.
^{187.} Rawat J., et al. Amino acid abundance and composition in cell culture medium affects trace metal tolerance and cholesterol synthesis. Biotechnol Prog. 2023; 39 (1): e3298.
^{188.} Pinto N. D. S., Brower M. Wide-surface pore microfiltration membrane drastically improves sieving decay in TFF-based perfusion cell culture and streamline chromatography integration for continuous bioprocessing. Biotechnol Bioeng. 2020; 117 (11): 3336-3344.
^{189.} Thyden Richard, et al. An Edible, Decellularized Plant Derived Cell Carrier for Lab Grown Meat. Applied Sciences. 2022; 12 (10): 5155.
^{190.} Lu Hongyun, et al. Bioprocessing by Decellularized Scaffold Biomaterials in Cultured Meat: A Review. Bioengineering. 2022; 9 (12): 787.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification and figures are considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize or be able to ascertain using no more than routine experimentation, many equivalents to the specific embodiments described herein. Such equivalents are intended to be encompassed by the following exemplary claims.

We claim:

1. A mammalian cell production system, comprising:
A) a living mammalian cell culture including a mammalian growth medium operative to feed nutrients and oxygen to a plurality of mammalian cells within the mammalian cell culture and to transport a mammalian waste from the plurality of mammalian cells;
B) a living algae cell culture including an algae growth medium operative to feed nutrients and carbon dioxide to a plurality of algae cells within the algae cell culture and to transport an algae waste from the plurality of algae cells;
wherein the living algae cell culture produces the algae waste with one or more nutrients and oxygen operative to feed the mammalian cell culture and wherein the living mammalian cell culture produces the mammalian waste with one or more nutrients and carbon dioxide operative to feed the algae cell culture; and
C) a mass exchange system operative to allow the mammalian growth medium to enter and/or to exit the living algae cell culture and to allow the algae growth medium to enter and/or to exit the living mammalian cell culture without a cross transfer of mammalian cells and algae cells;
wherein the system is capable of a continuous operation at a temperature in a range from about 30° C. to about 40° C. and a pH in a range from about 7 to about 8 thereby removing a need to cool down the algae growth medium and/or heat up the algae growth medium for a treatment of algae or for a replenishment of algae.

2. The mammalian cell production system of claim 1, wherein the system is capable of a continuous operation at a temperature at about 37° C.

3. The mammalian cell production system of claim 1, wherein the system is capable of a continuous operation at a pH at about 7.4.

4. The mammalian cell production system of claim 1, wherein the living algae cell culture is capable of receiving a light energy that is directed upon the plurality of algae cells.

5. The mammalian cell production system of claim 1, wherein the mammalian waste comprises urea, carbon dioxide, lactic acid, ammonia, or a combination thereof.

6. The mammalian cell production system of claim 1, wherein the algae waste comprises glucose, oxygen, or a combination thereof.

7. The mammalian cell production system of claim 1, further comprising a mammalian cell substrate or a mammalian cell carrier operative to provide an adherent growth substrate for a plurality of mammalian cells.

8. The mammalian cell production system of claim 1, further comprising:
a mammalian cell substrate or a mammalian cell carrier operative to provide an adherent growth substrate for a plurality of mammalian cells; and
wherein the plurality of mammalian cells include two or more mammalian cell types, and wherein the living mammalian cell culture is within a proliferation reactor that is in a fluid communication with the mass exchange system and with a conduit leading into a filter and a differentiation reactor;
wherein the filter provides a passage of a differentiated growth seed comprising an adherent growth of the two or more cell types from the proliferation reactor and into the differentiation reactor.

9. The mammalian cell production system of claim 1, further comprising:
a mammalian cell substrate or a mammalian cell carrier operative to provide an adherent growth substrate for a plurality of mammalian cells;
wherein the plurality of mammalian cells includes two or more mammalian cell types, and wherein the living mammalian cell culture is within a proliferation reactor that is in a fluid communication with the mass exchange system and with a conduit leading into a filter and a differentiation reactor;
wherein the filter provides a passage of a differentiated growth seed comprising an adherent growth of the two or more cell types from the proliferation reactor and into the differentiation reactor;
wherein the differentiation reactor is in fluid communication with the mass exchange system; and
the differentiation reactor provides a chamber for a differentiated growth suitable to grow into a differentiated cell populated scaffold; and the differentiation reactor includes an outlet operative to provide the differentiated cell populated scaffold.

10. A method for producing a tissue product, the method comprising the steps of:
(1) obtaining a mammalian cell production system, comprising:
A) a living mammalian cell culture including a mammalian growth medium operative to feed nutrients and oxygen to a plurality of mammalian cells within the mammalian cell culture and to transport a mammalian waste from the plurality of mammalian cells;
B) a living algae cell culture including an algae growth medium operative to feed nutrients and carbon dioxide to a plurality of algae cells within the algae cell culture and to transport an algae waste from the plurality of algae cells;
wherein the living algae cell culture produces the algae waste with one or more nutrients and oxygen operative to feed the mammalian cell culture and wherein the living mammalian cell culture produces the mammalian waste with one or more nutrients and carbon dioxide operative to feed the algae cell culture;
C) a mass exchange system operative to allow the mammalian growth medium to enter and/or to exit the living algae cell culture and to allow the algae growth medium to enter and/or to exit the living mammalian cell culture without a cross transfer of mammalian cells and algae cells;
(2) circulating the mammalian growth medium and the algae growth medium, whereby the mammalian growth medium and the algae growth medium enter and exit the mass exchange system; and
whereby the tissue product is produced by a growth of the living mammalian cell culture that is at least partially sustained by the algae cell culture.

11. The method of claim 10, wherein the system is capable of a continuous operation at a temperature in a range from about 30° C. to about 40° C. and a pH in a range from about 7 to about 8 thereby removing a need to cool down the algae growth medium and/or heat up the algae growth medium for a treatment of algae or for a replenishment of algae.

12. The method of claim 10, wherein the system is capable of a continuous operation at a temperature at about 37° C.

13. The method of claim 10, wherein the system is capable of a continuous operation at a pH at about 7.4.

14. The method of claim 10, further comprising a mammalian cell substrate or a mammalian cell carrier operative to provide an adherent growth substrate for a plurality of mammalian cells.

15. A method for recycling a mammalian cell culture medium, the method comprising the steps of:
(1) obtaining a mammalian cell production system, comprising:
A) a living mammalian cell culture including a mammalian growth medium operative to feed nutrients and oxygen to a plurality of mammalian cells within the mammalian cell culture and to transport a mammalian waste from the plurality of mammalian cells;
B) a living algae cell culture operative to utilize the mammalian growth medium to feed nutrients and carbon dioxide to a plurality of algae cells within the algae cell culture and to transport an algae waste from the plurality of algae cells;
wherein the living algae cell culture produces the algae waste with one or more nutrients and oxygen operative to feed the mammalian cell culture and wherein the living mammalian cell culture produces the mammalian waste with one or more nutrients and carbon dioxide operative to feed the algae cell culture;
wherein the system is capable of a continuous operation at a temperature in a range from about 30° C. to about 40° C. and a pH in a range from about 7 to about 8 thereby removing a need to cool down the algae growth medium and/or heat up the algae growth medium for a treatment of algae or for a replenishment of algae;
(2) extracting the algae waste from the plurality of algae cells; and
whereby a recycled mammalian cell culture medium is obtained from the algae waste.

16. The method of claim 15, further comprising the step of:
(1A) obtaining a mass exchange system operative to allow the mammalian growth medium to enter and/or to exit the living algae cell culture and to allow an algae growth medium to enter and/or to exit the living mammalian cell culture without a cross transfer of mammalian cells and algae cells.

17. The method of claim 15, wherein the system is capable of a continuous operation at a temperature at about 37° C.

18. The method of claim 15, wherein the system is capable of a continuous operation at a pH at about 7.4.

19. The method of claim 15, wherein the living algae cell culture is capable of receiving a light energy that is directed upon the plurality of algae cells.

20. The method of claim 15, wherein the mammalian waste comprises urea, carbon dioxide, lactic acid, ammonia, or a combination thereof.
